(19) United States
(12) Patent Application Publication
(10) Pub. No.: US 2011/0306125 A1
(43) Pub. Date: Dec. 15, 2011

(54) Protein Expression Methods

(75) Inventor: Marco Kessler, Danvers, MA (US)

(73) Assignee: Ironwood Pharmaceuticals, Inc., Cambridge, MA (US)

(21) Appl. No.: 13/001,888

(22) PCT Filed: Jun. 30, 2009
(86) PCT No.: PCT/US09/03879

§ 371 Date: Feb. 28, 2011

Related U.S. Application Data
(60) Provisional application No. 61/077,049, filed on Jun. 30, 2008.

(51) Int. Cl. C12N 5/10; C12N 15/63; C07K 14/00; C07K 1/14; C12N 1/21; C12N 1/19; C07K 1/107; C12N 15/11; C07K 7/08

(52) U.S. Cl. 435/348; 536/23.1; 435/320.1; 530/324; 530/326; 435/252.33; 435/254.2; 435/325; 435/358; 435/365; 530/345

(57) ABSTRACT

The present invention relates to compositions and methods for obtaining (e.g., expressing, isolating and/or purifying) polypeptides capable of binding to and/or activating the guanylate cyclase C receptor.

### PRIORITY CLAIM

This application claims priority to U.S. Application Ser. No. 61/077,049, filed Jun. 30, 2008. The entire contents of the aforementioned application is incorporated herein by reference.

### TECHNICAL FIELD

This disclosure relates to compositions and methods for obtaining (e.g., expressing, isolating and/or purifying) polypeptides capable of binding to and/or activating the guanylate cyclase C receptor.

### SEQUENCE LISTING

This application incorporates by reference in its entirety the Sequence Listing entitled IW060 seq_ST25.txt (5 megabytes) which was created Jun. 30, 2009 and filed with herewith in this International PCT application on Jun. 15, 2009.

### BACKGROUND

The guanylate cyclase (GC-C) receptor is a key regulator of fluid and electrolyte balance in the intestine. When stimulated, this receptor, which is located on the apical membrane of the intestinal epithelial surface, causes an increase in intestinal epithelial cyclic GMP (cGMP). This increase in cGMP is believed to cause a decrease in water and sodium absorption and an increase in chloride and potassium ion secretion, leading to changes in intestinal fluid and electrolyte transport and increased intestinal motility. The intestinal GC-C receptor possesses an extracellular ligand binding region, a transmembrane region, an intracellular protein kinase-like region and a cyclase catalytic domain. Proposed functions for the GC-C receptor are fluid and electrolyte homeostasis, the regulation of epithelial cell proliferation and the induction of apoptosis (Shalubhai 2002 Curr Opin Drug Dis Devel 5:261-268).

In addition to being expressed in the intestine by gastrointestinal epithelial cells, the GC-C receptor is expressed in extra-intestinal tissues including kidney, lung, pancreas, pituitary, adrenal, developing liver and gall bladder (reviewed in Vaandrager 2002 Mol Cell Biochem 230:73-83, Kulaksiz et al. 2004, Gastroenterology 126:732-740) and male and female reproductive tissues (reviewed in Vaandrager 2002 Mol Cell Biochem 230:73-83).

A number of naturally occurring polypeptides are capable of binding to and/or activating the GC-C receptor. In humans, such polypeptides include, for example, guanylin (Gn), uroguanylin (Ugn), lymphoguanylin, renoguanylin (each of which are considered to be members of the natriuretic family of peptides (see e.g., Currie et al., Proc. Natl. Acad. Sci. USA., 89:947-951, 1992; Hamra et al., Proc. Natl. Acad. Sci. USA., 90:10464-10468, 1993; Yuge et al., J. Biol. Chem., 278:22726-22733, 2003; Forte et al., Endocrinology, 140:1800-1806, 1999)), and the class of bacterially derived peptides, termed ST (reviewed in Gianella 1995 J Lab Clin Med 125:173-181). Such polypeptides are typically referred to as GC-C agonists.

Effective systems are required for obtaining (e.g., expressing and purifying) GC-C agonists capable of binding to and/or activating GC-C.

### SUMMARY

The present disclosure provides compositions and methods for producing biologically active GC-C receptor binding and/or activating peptides.

Described herein is a nucleic acid molecule comprising (consisting essentially of or consisting of) a nucleotide sequence encoding a polypeptide comprising (consisting essentially of or consisting of) the amino acid sequence:

A-B-C-D-E, wherein:

A comprises a GC-C receptor binding polypeptide presequence;

B is one or more methionine residues;

C comprises a GC-C receptor binding polypeptide prosequence;

D is one or more methionine residues; and

E comprises a GC-C receptor binding polypeptide.

Also described herein is a nucleic acid molecule comprising (consisting essentially of or consisting of) a nucleotide sequence encoding a polypeptide comprising (consisting essentially of or consisting of) the amino acid sequence:

B-C-D-E, wherein:

B is one or more methionine residues;

C comprises a GC-C receptor binding polypeptide prosequence;

D is one or more methionine residues; and

E comprises a GC-C receptor binding polypeptide.

A nucleic acid molecule comprising (consisting essentially of or consisting of) a nucleotide sequence encoding a polypeptide comprising (consisting essentially of or consisting of) the amino acid sequence:

A-C-D-E, wherein:

A comprises a GC-C receptor binding polypeptide presequence;

C comprises a GC-C receptor binding polypeptide prosequence;

D is one or more methionine residues; and

E comprises a GC-C receptor binding polypeptide.

Described herein is a nucleic acid molecule comprising (consisting essentially of or consisting of) a nucleotide sequence encoding a polypeptide comprising (consisting essentially of or consisting of) the amino acid sequence:

A-B-C-E, wherein:

A comprises a GC-C receptor binding polypeptide presequence;

B is one or more methionine residues;

C comprises a GC-C receptor binding polypeptide prosequence;

E comprises a GC-C receptor binding polypeptide.

Described herein is a nucleic acid molecule comprising (consisting essentially of or consisting of) a nucleotide sequence encoding a polypeptide comprising (consisting essentially of or consisting of) the amino acid sequence:

C-D-E, wherein:

C comprises a GC-C receptor binding polypeptide prosequence;

D is one or more methionine residues; and

E comprises a GC-C receptor binding polypeptide.

Described herein is a nucleic acid molecule comprising (consisting essentially of or consisting of) a nucleotide sequence encoding a polypeptide comprising (consisting essentially of or consisting of) the amino acid sequence:

A-B-E, wherein:

A comprises a GC-C receptor binding polypeptide presequence;

B is one or more methionine residues;

E comprises a GC-C receptor binding polypeptide.

Described herein is a nucleic acid molecule comprising (consisting essentially of or consisting of a nucleotide sequence encoding a polypeptide comprising (consisting essentially of or consisting of) the amino acid sequence:

D-E, wherein:

D is one or more methionine residues; and

E comprises a GC-C receptor binding polypeptide.

In various cases: B, when present, is one methionine; B, when present, is two or more methionines; D, when present, is one methionine; D, when present, is two or more methionines; A, when present, comprises a sequence selected from SEQ ID NO:17, SEQ ID NO:18, SEQ ID NO:19, SEQ ID NO:20, SEQ ID NO:21, SEQ ID NO:22, or a presequence depicted in FIG. 10; A is SEQ ID NO:17; A is SEQ ID NO:18; A is SEQ ID NO:20; A is SEQ ID NO:21; A is SEQ ID NO:22; A is a presequence depicted in FIG. 10; A is a presequence depicted in FIG. 9; C, when present, comprises a sequence chosen from a pro sequence depicted in FIG. 10 or FIG. 9, SEQ ID NO:11, SEQ ID NO:12, SEQ ID NO:13, or SEQ ID NO:14; C is SEQ ID NO:11; C is SEQ ID NO:12; C is SEQ ID NO:13; wherein C is SEQ ID NO:14; C is a prosequence depicted in FIG. 10; C is a prosequence depicted in FIG. 9; E comprises a sequence selected from: a processed active peptide (mature) sequence depicted in FIG. 10, SEQ ID NO:9, SEQ ID NO:10, SEQ ID NO:14A; SEQ ID NO:199; SEQ ID NO:15, or SEQ ID NO:16; E is a processed active peptide (mature) sequence depicted in FIG. 10; E is SEQ ID NO:9; E is SEQ ID NO:10; E is SEQ ID NO:14A; E is SEQ ID NO:199; E is SEQ ID NO:15; E is SEQ ID NO:16; E comprises a sequence chosen from the group consisting of SEQ ID NOs: 27-33, 34-59 and 63-62; E is SEQ ID NO: 63; E is SEQ ID NO: 64; E is SEQ ID NO: 805.

In some cases: E comprises: E1-E2-E3, wherein E1 is an N-terminal non-core sequence in FIG. 9, E2 is an active “core” sequence in FIGS. 9 and E3 is a C-terminal non-core sequence in FIG. 9; E comprises: E1-E1′-E2-E3, wherein E1 is an N-terminal non-core sequence in FIG. 9, E1′ is one or more methionine residues, E2 is an active “core” sequence in FIGS. 9 and E3 is a C-terminal non-core sequence in FIG. 9; E1′ is one methionine residue; E1′ is more than one methionine residue; E comprises E1-E2, wherein: E1 is an N-terminal non-core sequence in FIGS. 9 and E2 is an active “core” sequence in FIG. 9; E comprises E2-E3, wherein: E2 is an active “core” sequence in FIGS. 9 and E3 is a C-terminal non-core sequence in FIG. 9; E comprises E2, wherein E2 is an active “core” sequence in FIG. 9; E comprises a sequence chosen from a sequence depicted in FIG. 10.

Also described is a nucleic acid molecule comprising (consisting essentially of or consisting of) a nucleotide sequence encoding a polypeptide comprising (consisting essentially of or consisting of) the amino acid sequence: Z2-Z3, wherein: Z2 is one or more methionine residues and Z3 comprises SEQ ID NO:9, SEQ ID NO:10, SEQ ID NO:15, or SEQ ID NO:16. In certain cases, the polypeptide further comprises Z0 or Z1 or both, wherein: Z0 is SEQ ID NO:17, SEQ ID NO:18, SEQ ID NO:19, SEQ ID NO:20, SEQ ID NO:21, SEQ ID NO:22, a pre sequence depicted in FIG. 9, or is missing; and Z1 comprises SEQ ID NO:11, SEQ ID NO:12, SEQ ID NO:13, or SEQ ID NO:14, a pro sequence depicted in FIG. 9, or is missing. In various cases: the polypeptide comprises Z1 Z2 Z3; the polypeptide comprises Z0 Z2 Z3; the polypeptide comprises Z0 Z1 Z2 Z3; the polypeptide comprises Z0 Z2 Z1 Z2 Z3, wherein: Z0 is SEQ ID NO:17, SEQ ID NO:18, SEQ ID NO:19, SEQ ID NO:20, SEQ ID NO:21, SEQ ID NO:22, a pre sequence depicted in FIG. 9, or is missing and Z1 comprises SEQ ID NO:11, SEQ ID NO:12, SEQ ID NO:13, or SEQ ID NO:14, a pro sequence depicted in FIG. 9, or is missing; the polypeptide comprises Z0 Z2 Z1 Z3, wherein: Z0 is SEQ ID NO:17, SEQ ID NO:18, SEQ ID NO:19, SEQ ID NO:20, SEQ ID NO:21, SEQ ID NO:22, a pre sequence depicted in FIG. 9, or is missing and Z1 comprises SEQ ID NO:11, SEQ ID NO:12, SEQ ID NO:13, or SEQ ID NO:14, a pro sequence depicted in FIG. 9, or is missing; Z0 comprises SEQ ID NO:17; Z0 comprises SEQ ID NO:18; Z0 comprises SEQ ID NO:19; Z0 comprises SEQ ID NO:20; Z0 comprises SEQ ID NO:21; Z0 comprises SEQ ID NO:22; Z0 comprises a pre sequence depicted in FIG. 9; Z1 comprises SEQ ID NO:11; Z1 comprises SEQ ID NO:12; Z1 comprises SEQ ID NO:13; Z1 comprises SEQ ID NO:14; Z1 comprises a pro sequence depicted in FIG. 9; Z3 comprises SEQ ID NO:9; Z3 comprises SEQ ID NO:10; Z3 comprises SEQ ID NO:15; Z3 consists of SEQ ID NO:16.

Also described is a nucleic acid molecule comprising (consisting essentially of or consisting of) a nucleotide sequence encoding a polypeptide comprising (consisting essentially of or consisting of) the amino acid sequence:

A′-B′-Z2-D′, wherein:

A′ is an amino acid sequence comprising (consisting essentially of or consisting of) a pre sequence depicted in FIG. 9, or is missing;

B′ is an amino acid sequence comprising (consisting essentially of or consisting of) a pro sequence depicted in FIG. 9, or is missing;

Z2 is one or more methionine residues; and

D′ is an amino acid sequence selected from the group consisting of SEQ ID NOs:63-1629, or an active core sequence depicted in FIG. 9.

Also described is a nucleic acid molecule comprising (consisting essentially of or consisting of) a nucleotide sequence encoding a polypeptide comprising (consisting essentially of or consisting of) the amino acid sequence:

A′-Z2-D′, wherein:

A′ is an amino acid sequence comprising (consisting essentially of or consisting of) a pre sequence depicted in FIG. 9, or is missing;

Z2 is one or more methionine residues; and

D′ is an amino acid sequence selected from the group consisting of SEQ ID NOs:63-1629, or an active core sequence depicted in FIG. 9.

Also described is a nucleic acid molecule comprising (consisting essentially of or consisting of) a nucleotide sequence encoding a polypeptide comprising (consisting essentially of or consisting of) the amino acid sequence:

B′-Z2-D′, wherein:

B′ is an amino acid sequence comprising (consisting essentially of or consisting of) a pro sequence depicted in FIG. 9, or is missing;

Z2 is one or more methionine residues; and

D′ is an amino acid sequence selected from the group consisting of SEQ ID NOs:63-1629, or an active core sequence depicted in FIG. 9.

Also described is a nucleic acid molecule comprising (consisting essentially of or consisting of) a nucleotide sequence encoding a polypeptide comprising (consisting essentially of or consisting of) an amino acid sequence depicted in Table 3, wherein:

A′ is an amino acid sequence comprising (consisting essentially of or consisting of) a pre sequence depicted in FIG. 9, or is missing;

B′ is an amino acid sequence comprising (consisting essentially of or consisting of) a pro sequence depicted in FIG. 9, or is missing;

Z2 is one or more methionine residues; and

D′ is an amino acid sequence selected from the group consisting of SEQ ID NOs:63-1629, or an active core sequence depicted in FIG. 9.

In various cases: the nucleic acid molecule further comprises a nucleotide sequence encoding amino acid sequences C′, E′, or both C′ and E′, wherein C′ is located between Z2 and D′ and/or E′ is located adjacent to and following D′; C′ is an amino acid sequence comprising (consisting essentially of or consisting of) an amino-terminal non-core sequence depicted in FIG. 9; E′ is an amino acid sequence comprising (consisting essentially of or consisting of) a carboxy-terminal non-core depicted in FIG. 9; D′ is SEQ ID NO:63; D′ is SEQ ID NO:64; D′ is SEQ ID NO:805.

Also described herein are: a nucleic acid molecule comprising (consisting essentially of or consisting of) a nucleotide sequence encoding a polypeptide comprising (consisting essentially of or consisting of) SEQ ID NO:7; a nucleic acid molecule comprising (consisting essentially of or consisting of) a nucleotide sequence encoding a polypeptide comprising (consisting essentially of or consisting of) SEQ ID NO:8; a nucleic acid molecule comprising (consisting essentially of or consisting of) the nucleotide sequence of SEQ ID NO:24; a nucleic acid molecule comprising (consisting essentially of or consisting of) the nucleotide sequence of SEQ ID NO:2; an a nucleic acid molecule comprising (consisting essentially of or consisting of) the nucleotide sequence of SEQ ID NO:4. In certain cases: the polypeptide comprises an affinity tag located at the amino-terminus and/or the carboxy-terminus of the polypeptide; the polypeptide and the affinity protein are adjacent to each other; the polypeptide and the affinity protein are separated by a protease recognition site.

In some cases the nucleic acid molecule further comprises: a nucleic acid sequence encoding an affinity tag located at the 5′ or 3′ terminus of the nucleic acid molecule; and a nucleic acid sequence encoding a protease recognition site.

Also described is an expression vector comprising (consisting essentially of or consisting of) a nucleic acid molecule described herein; an expression vector comprising (consisting essentially of or consisting of) two or more nucleic acid molecules; an expression vector wherein the nucleic acid molecule is operably linked to a promoter capable of driving expression of the nucleic acid molecule in a cell; an expression vector wherein the nucleic acid molecule is operably linked to a promoter capable of driving expression of the nucleic acid molecule in a cell; an expression vector further comprising (consisting essentially of or consisting of) an internal ribosome entry site; a DNA vector; expression vector pET32b.

Also described is a polypeptide encoded by the nucleic acid molecule described herein. In certain cases: the polypeptide consists of one methionine residue; the polypeptide consists of two methionine residues; and Z2 comprises two or more methionine residues.

Also described is a recombinant cell comprising (consisting essentially of or consisting of) a nucleic acid molecule or expression construct described herein.

Also described is a method of making a polypeptide capable of binding to and/or activating the guanylate cyclase (GC-C) receptor, the method comprising (consisting essentially of or consisting of) obtaining a polypeptide described herein and cleaving the polypeptide at carboxyterminal to methionine residues. In certain cases: the step of cleaving the polypeptide comprises contacting the polypeptide with cyanogen bromide; and the polypeptide capable of binding to and/or activating the guanylate cyclase (GC-C) receptor is purified.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosed methods and compositions will be apparent from the following detailed description and figures, and from the claims.

### DESCRIPTION OF DRAWINGS

FIG. 1 shows the nucleotide sequence of human uroguanylin, including portions coding for the pre and pro sequences (GenBank® U34279; GI:1236798) (SEQ ID NO:1).

FIG. 2 shows the nucleotide sequence of human uroguanylin, including portions coding for the pre and pro sequences modified to encode a methionine just before the sequence encoding the mature protein (SEQ ID NO:2). The added methione codon is in bold and underlined.

FIG. 3 shows the nucleotide sequence of human guanylin (GenBank® Accession M97496; GI:183414) (SEQ ID NO:3).

FIG. 4 shows the nucleotide sequence of human guanylin modified to encode a methionine just before the sequence encoding the mature protein (SEQ ID NO: 4). The added methione codon is in bold and underlined.

FIG. 5 shows the huProMUGN DNA cassette. The nucleic acid sequence is SEQ ID NO:23. The guanylin coding sequence has been adapted for codon usage in E. coli. The amino acid sequence is SEQ ID NO:24. Restriction endonuclease sites are indicated in bold or using (˜). The PreScission3 protease cleavage site is indicated by an open box. The cleavage site within this site is represented by a thin arrow. The cleavage site amino acids that will remain following PreScission3 protease cleavage are shown by a circle. The Pro UGN nucleotide and amino acid sequence is indicated by a solid underline. The mature UGN nucleotide and amino acid sequence is indicated by a dotted underline. The methionine inserted between the pro and mature UGN sequences is indicated by an arrow.

FIG. 6 is a schematic representation of the Trx-huProMUGN fusion protein. TrxA, HisTag, and S Tag are affinity tags. PreScission is a protease recognition site. HuProMUGN is modified human prouroguanylin (i.e. comprising an additional methionine residue).

FIG. 7 is a photograph of a coomassie stained gel. Molecular weight markers are shown on the left (M). Lane 1 is following cleavage with PreScission protease and Glutathione sepharose chromatography. Lane 2 is flow through (Trx-proMuroguanylin and proMuroguanylin). Lane 3 is the eluate following treatment with PreScission protease and purification using TALON metal affinity chromatography. Lane 4 is proMuroguanylin. Lane 5 is TrxA tag and uncleaved fusion protein.

FIG. 8 is a line graph showing cGMP activity. Sigma UGN is synthetic uroguanylin.

FIG. 9 is a table depicting various subsequences of ST polypeptides.

FIG. 10 is a table depicting various subsequences of guanylin and uroguanylin polypeptides

### DETAILED DESCRIPTION

The present disclosure provides compositions and methods for producing polypeptides capable of binding to and/or activating the guanylate cyclase (GC-C) receptor.

### Polypeptides

In some cases the polypeptides described herein are produced as a mature polypeptide. In some cases, the polypeptides described herein are obtained as propeptides and/or prepropeptides and/or prepeptides. These pro, pre and/or prepropeptides can be processed using the methods described herein to yield a smaller polypeptide. Such polypeptides may be referred to as “mature,” “active,” and/or “biologically active” polypeptides. The activity of a mature peptide or a biologically active peptide can be assessed using one or more of the methods described herein. The biologically active peptides bind the GC-C receptor and/or bind and activate the GC-C receptor (GC-C receptor agonists).

In some cases, the peptides described herein can be expressed as fusion proteins, e.g., with an amino (N)-terminal and/or carboxy (C)-terminal affinity tag. Suitable affinity tags and the methods required for producing a fusion protein that includes an affinity tag are known in the art and are included in those described below.

In some cases a polypeptide will be produced, e.g., recombinantly, with a pre sequence, a pro sequence, and/or non-core sequences (e.g., an N-terminal portion of the mature peptide that is not essential for activity (N-terminal non-core sequence) and/or a C-terminal portion of the mature peptide that is not essential for activity (C-terminal non-core sequence)). In certain cases the one or more of the pre sequence, pro sequence, N-terminal non-core sequence and/or C-terminal non-core sequence is removed prior to administering the polypeptide to a patient. In certain cases only pre sequence and/or pro sequence is removed prior to administration of the polypeptide to a patient. In other cases the preproprotein, proprotein or the preprotein is administered to the patient. The pre sequence and/or the pro sequence may stabilize the polypeptide or an active isomer thereof, facilitate efficient folding of the polypeptide or desired intracellular (e.g. export to the periplasm) or extracellular localization during recombinant synthesis, or protect the polypeptide from degradation in the patient's body. Thus, pre sequences, pro sequences and/or preprosequences that do not significantly interfere with GC-C receptor agonist activity can be beneficial. The pre sequence can be chosen from those including but not limited to the pre sequences described herein. In certain cases the pre sequence may comprise a positively charged amino terminal region, followed by a hydrophobic region, and a neutral but polar carboxy terminal region. Such sequences can be predicted using algorithms such as SignalP 3.0 (Emanuelsson et al. (2007) Nature Protocols 2: 953-971; and www.cbs.dtu.dk/services/SignalP/).

Guanylin and Uroguanylin and Related Peptides

GN and UGN are guanylate cyclase activating peptide hormones that are secreted from the epithelia of the intestine, kidney, pancreas, and salivary gland. Specifically, enterochromaffin cells along the intestine secrete GN and UGN into the intestinal lumen (Cetin et al., Proc. Natl. Acad. Sci. USA., 91:2935-2939, 1994; Perkins et al., Gastroenterology, 113:1007-1014, 1997). When correctly expressed, these peptides bind to and/or activate the guanylate cyclase C receptor (GC-C receptor).

Normally, GN and UGN are secreted as biologically inactive prohormones (i.e., the prohormone is nearly inactive with respect to GC-C activation), which are processed to yield the mature, biologically active, hormones. GN prohormone (proguanylin (proGN)) has 94 amino acids and is processed to yield 15 amino acid mature guanylin. UGN prohormone (prouroguanylin (proUGN)) has 86 amino acid residues and is processed in the kidney to yield a 16 amino acid peptide (e.g., found in urine) and in the ciculation to yield a 24 amino acid peptide (e.g., found in blood).

The amino-terminal prosequences of proGN and proUGN have intra-molecular chaperone-like features. Proper biological activity requires that cysteine residues within the mature hormone form disulphide bonds in a 1-3/2-4 conformation (Forte and Currie, FASEB J., 9:643-650, 1995; Klodt et al., J. Pept. Res., 50:222-230, 1997). Two topological isoforms of each peptide exists (known as A- and B-isomers), with only the A-isomer showing biological activity (Lauber and Marx, Protein and Peptide Letters, 12:153-158, 2005).

Unmodified human prouroguanylin has the sequence: VYIQYQGFRVQLESMKKLSDLEAQWAPSPRLQAQSLLPAVCHHPALPQDLQPVCAS QEASSIFKTLRTIANDDCELCVNVACTGCL (SEQ ID NO:5-human proUGN (huproUGN); mature kidney processed UGN portion (16 amino acids) is shown underlined; mature circulation processed UGN (24 amino acids) is shown in bold).

Unmodified human proguanylin has the sequence:

VTVQDGNFSFSLESVKKLKDLQEPQEPRVGKLRNFAPIPGEPVVPILCSNPNFP EELKPLCKEPNAQEILQRLEEIAEDPGTCEICAYAACTGC (SEQ ID NO:6; human proGN (huproGN); mature GN portion underlined).

Described herein are various useful polypeptides that can include all or a portion of the sequence of human prouroguanylin. These polypeptides can be peptides encoded by all or a portion of the sequence of SEQ ID NO:1 that has been modified to include one or more methionine encoding codons (ATG). For example, peptides can include those encoded by the sequence of SEQ ID NO:2.

In some cases, peptides can include all or a portion of a modified proUGN that has one or more methionine residues inserted immediately prior to the first amino acid of the 16 residue mature UGN peptide (i.e., immediately prior to the underlined portion of SEQ ID NO: 5 for the kidney form) or one or more methionine residues inserted immediately prior to the first amino acid of the 24 residue mature UGN peptide (i.e., immediately prior to the bold portion of SEQ ID NO: 5 for the mature circulation form). Thus, certain peptides can include all or a portion of the sequence: VYIQYQGFRVQLESMKKLSDLEAQWAPSPRLQAQSLLPAVCHHPALPQDLQPVCAS QEASSI [symbol] FKTLRTIA [symbol] NDDCELCVNVACTGCL (SEQ ID NO:7—modified human prouroguanylin (huproMUGN); 16 residue mature (kidney form) uroguanylin portion is underlined; 24 residue mature (circulation form) uroguanylin portion is shown in bold; one or more inserted methionines are shown in a box).

Also described herein are various peptides that include all or a portion of a modified proGN that has one or more methionine residues inserted immediately prior to the first amino acid of the mature GN peptide (i.e., just before the underlined portion of SEQ ID NO: 6). Thus, certain peptides include all or a portion of the sequence: VTVQDGNFSFSLESVKKLKDLQEPQEPRVGKLRNFAPIPGEPVVPILCSNPNFPEELKP LCKEPNAQEILQRLEEIAED [symbol] PGTCEICAYAACTGC (SEQ ID NO:8; modified human proguanylin (huproMGN); mature guanylin portion underlined; inserted methionine is shown in a box).

In some cases, peptides can include all or a portion of a peptide that is, or is related to, the mature uroguanylin portion of human prouroguanylin. For example, peptides can include all or a portion of the sequences:

NDDCELCVNVACTGCL
(SEQ ID NO: 9; unmodified mature uroguanylin);
or
(SEQ ID NO: 10)
X₇₃ D X₇₅ C E L C X₈₀ N V AC₈₄T₈₅G₈₆C₈₇L₈₈,

wherein:

X₇₃ is N, T, G or Q;

X₇₅ is D or E;

X₈₀ is V or I;

C₈₄ is present or absent;

T₈₅ is present or absent;

G₈₆ is present or absent;

C₈₇ is present or absent; and

L₈₈ is present or absent; in combination with all or a portion of the human proUGN prosequence. Thus, peptides can include a peptide having all or a portion of the sequence:

(SEQ ID NO: 11)
VYIQYQGFRVQLESMKKLSDLEAQWAPSPRLQAQSLLPAVCHHPALPQ
DLQPVCASQEASSIFKTLRTIA.

In some cases, peptides can include all or a portion of a peptide that is related to the prosequence of human prouroguanylin. Thus, peptides include a peptide comprising (or consisting of or consisting essentially of) at least 10 contiguous amino acid of a polypeptide having the sequence:

(SEQ ID NO: 12)
V X₂ I X₄ Y X₆ G X₈ X₉ V X₁₁ L X₁₃ S X₁₅ K X₁₇ L X₁₉
X₂₀ L X₂₂ X₂₃ X₂₄ X₂₅ X₂₆ X₂₇ X₂₈ X₂₉ X₃₀ X₃₁ X₃₂
X₃₃ X₃₄ X₃₅ X₃₆ X₃₇ X₃₈ X₃₉ X₄₀ X₄₁ C X₄₃ X₄₄ X₄₅
A L P X₄₉ D L X₅₂ P X₅₄ C X₅₆ X₅₇ X₅₈ X₅₉ X₆₀ X₆₁
X₆₂ X₆₃ X₆₄ X₆₅ X₆₆ LR X₆₉ X₇₀ X₇₁,

wherein:

X₂ is Y or D;

X₄ is Q or K;

X₆ is Q, H or E;

X₈ is F or Y;

X₉ is R or Q;

X₁₁ is Q or K;

X₁₃ is E, K or D;

X₁₅ is M or V;

X₁₇ is K or Q;

X₁₉ is S, N, K or D;

X₂₀ is D, E or A;

X₂₂ is E, V or L;

X₂₃ is A, E or G;

X₂₄ is Q or K;

X₂₅ is W, Q, E or P;

X₂₆ is A, M, V or R;

X₂₇ is P or S;

X₂₈ is S, N, D or F;

X₂₉ is P or R;

X₃₀ is R, Q, G or H;

X₃₁ is L, P, Q or R;

X₃₂ is Q, R or M;

X₃₃ is A, K, R, D or G;

X₃₄ is Q, S or T;

X₃₅ is S, G, D or Q;

X₃₆ is L, R or is missing;

X₃₇ is L, P or D;

X₃₈ is L, Q or P;

X₃₉ is P or S;

X₄₀ is A, S, D or V;

X₄₁ is V or L;

X₄₃ is H, Y or S;

X₄₄ is H, N or D;

X₄₅ is P or S;

X₄₉ is Q, L, P or S;

X₅₂ is Q or R;

X₅₄ is V or I;

X₅₆ is A, Q, T or E;

X₅₇ is S or N;

X₅₈ is Q, E, K or S;

X₅₉ is E, D or Q;

X₆₀ is A or V;

X₆₁ is S or A;

X₆₂ is S or N;

X₆₃ is I or T;

X₆₄ is F or L;

X₆₅ is K, Q or L;

X₆₆ is T or A;

X₆₉ is T or S;

X₇₀ is I or M; and

X₇₁ is A, S or D.

In some cases, peptides can include all or a portion of the human proGN prosequence. Thus, peptides can include peptides having the sequence:

(SEQ ID NO: 13)
VTVQDGNFSFSLESVKKLKDLQEPQEPRVGKLRNFAPIPGEPVVPILCSN
PNFPEELKPLCKEPNAQEILQRLEEIAED.

In some cases, peptides can include all or a portion of SEQ ID NO:15933 that is related to the prosequence of human proguanylin. Thus, the peptides disclosed herein include peptides comprising (or consisting of or consisting essentially of) at least 10 contiguous amino acid of a polypeptide having the sequence:

(SEQ ID NO: 15933)
X₁ X₂ X₃ X₄ X₅ X₆ X₇ X₈ X₉ X₁₀ X₁₁ L E X₁₄ V K X₁₇
L X₁₉ X₂₀ L X₂₂ X₂₃ X₂₄ X₂₅ X₂₆ X₂₇ X₂₈ X₂₉ X₃₀ X₃₁
X₃₂ X₃₃ X₃₄ X₃₅ X₃₆ X₃₇ X₃₈ X₃₉ X₄₀ X₄₁ X₄₂ X₄₃ X₄₄
X₄₅ X₄₆ X₄₇ X₄₈ X₄₉ X₅₀ C X₅₂ X₅₃ X₅₄ X₅₅ X₅₆ X₅₇ P
X₅₉ X₆₀ X₆₁ X₆₂ P X₆₄ X₆₆ X₆₇ X₆₈ X₆₉ X₇₀ X₇₁ X₇₂
X₇₃ X₇₄ X₇₅ R L X₇₈ X₇₉ X₈₀ X₈₁ X₈₂ X₈₃

wherein:

X₁ is V or S;

X₂ is T, L, I, Y or E;

X₃ is V or F;

X₄ is Q or K;

X₅ is D or E;

X₆ is G or N;

X₇ is D, N, E or G;

X₈ is For L;

X₉ is S, T or K;

X₁₀ is F or Y;

X₁₁ is S or P;

X₁₄ is S or A;

X₁₇ is K, Q or R;

X₁₉ is K or H;

X₂₀ is D, E, A, H or G;

X₂₂ is Q, R, G, M, A;

X₂₃ is E, Q or D;

X₂₄ is A, S, E, V, L or P;

X₂₅ is Q, N, P, G or S;

X₂₆ is E, K, M or V;

X₂₇ is G, L or is missing;

X₂₈ is Q, S, R, A or is missing;

X₂₉ is E, K, S, A or is missing;

X₃₀ is P, V, M or A;

X₃₁ is R, Q, T, I, A or is missing;

X₃₂ is L, V, I, G, N or S;

X₃₃ is P, G, R, V, M, A, P;

X₃₄ is S, R or K;

X₃₅ is H, L, I, N or K;

X₃₆ is R, K or is missing;

X₃₇ is N, K or is missing;

X₃₈ is F or is missing;

X₃₉ is A or is missing;

X₄₀ is P, L or is missing;

X₄₁ is I, R or is missing;

X₄₂ is L, P, F, V, R or is missing;

X₄₃ is G, V, D, P, L, A or is missing;

X₄₄ is G, E, K, A, Q, R or S;

X₄₅ is P, S, H or K;

X₄₆ is V, I, P, A or Q;

X₄₇ is A, V, I, A, L, G or T;

X₄₈ is P, A, S, Y or is missing;

X₄₉ is I, Q, V, N, G, E, H, S or F;

X₅₀ is L, A or P;

X₅₂ is S, N, A, Q or G;

X₅₃ is S or missing;

X₅₄ is H, N, D, S, L, F, or Q;

X₅₅ is P, S, L or K;

X₅₆ is A, K, N, T, G, or Q;

X₅₇ is For L;

X₅₉ is E, K or Q;

X₆₀ is E, A or D;

X₆₁ is L or F;

X₆₂ is K, R, Q or L;

X₆₄ is L, I or V;

X₆₆ is K, E, Q, T or R;

X₆₇ is E, K, R or Q;

X₆₈ is P, S, E or R;

X₆₉ is N, D or G;

X₇₀ is A or S;

X₇₁ is E, Q, P, A or S;

X₇₂ is E, D, Q, M or A;

X₇₃ is I, A, or S;

X₇₄ is L, F or V;

X₇₅ is Q, E, D, N, G or A;

X₇₈ is E, A, G or C;

X₇₉ is E, A, V, S, L or M;

X₈₀ is I or V;

X₈₁ is A or P;

X₈₂ is E, Q, A or S;

X₈₃ is D or E; and wherein the mature and prosequences are separated by one or more methionine residues.

In some cases, the peptides described herein can include all or a portion of a peptide that is, or is related to, circulating uroguanylin. Thus, the peptides disclosed herein can include peptides having the sequence:

| | |
|---|---|
| FKTLRTIANDDCELCVNVACTGCL. | (SEQ ID NO: 14) |

In some cases, peptides can include all or a portion of SEQ ID NO:199. Thus, the peptides disclosed herein include a peptides comprising (or consisting of or consisting essentially of) at least 10 contiguous amino acid of a polypeptide having the sequence (SEQ ID NO:199):

X₁ X₂ X₃ X₄ X₅ L R X₆ X₇ X₈ Z₂ X₉ D X₁₀ C E L C X₁₁
N V AC₁₂T₁₃G₁₄C₁₅L₁₆,

wherein:

X₁ is S or N or is absent;

X₂ is I or T or is absent;

X₃ is F or L or is absent;

X₄ is K, Q or L or is absent;

X₅ is T or A or is absent;

X₆ is T or S or is absent;

X₇ is I or M or is absent;

X₈ is A, S or D or is absent;

Z₂ is one or more M or is absent;

X₉ is N, T, G or Q;

X₁₀ is D or E;

X₁₁ is V or I;

C₁₂ is present or absent;

T₁₃ is present or absent;

G₁₄ is present or absent;

C₁₅ is present or absent; and

L₁₆ is present or absent.

In some cases, the peptides can include all or a portion of a peptide that is, or is related to, the mature guanylin portion of human proguanylin. For example, useful polypeptides can include all or a portion of the sequences:

(SEQ ID NO: 15)
P G T C E I C A Y A A C T G C X₁₀₀,

wherein

X₁₀₀ is F or is missing; or

(SEQ ID NO: 16)
P X₈₆ X₈₇ C E I C A X₉₃ A A C X₉₇ G C X₁₀₀,

wherein

X₈₆ is G, S, R, or N;

X₈₇ is S or T;

X₉₃ is Y or F;

X₉₇ is T or A; and

X₁₀₀ is F or is missing.

In some cases, the peptides described herein include peptides or salts thereof comprising the amino acid sequence:

A₁′-B₁′-C₁′, wherein:

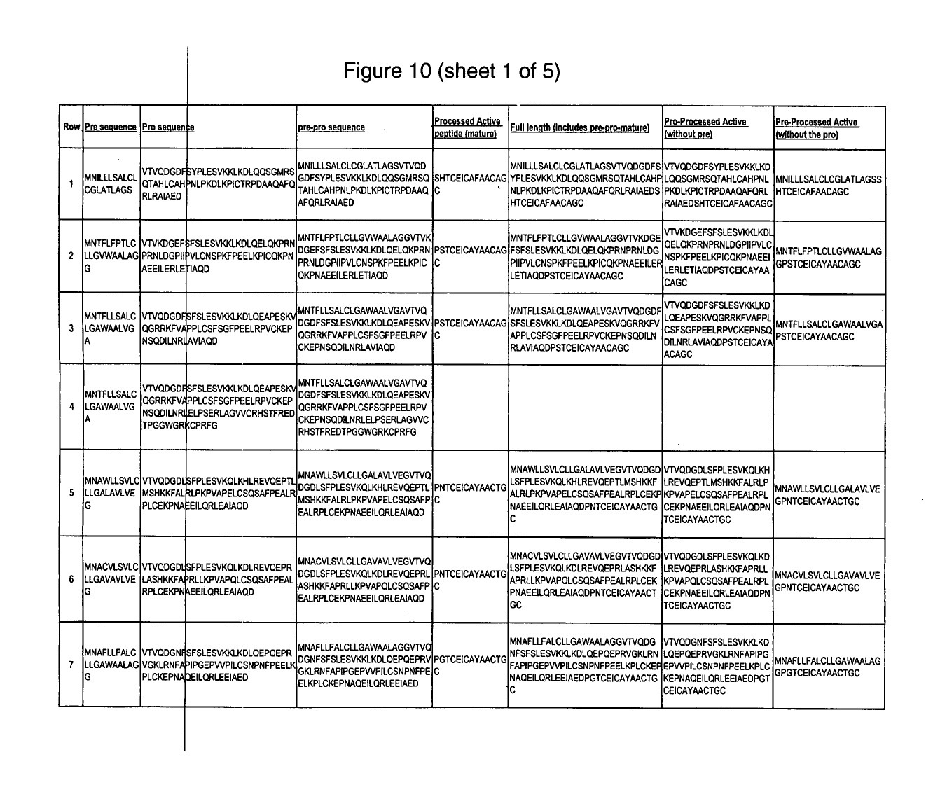

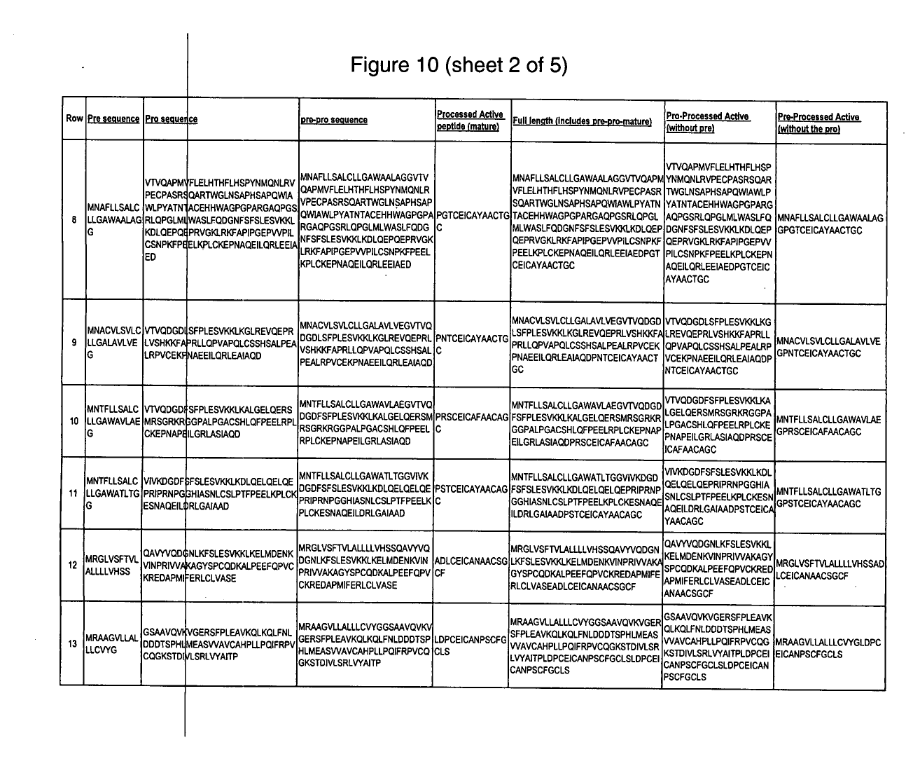

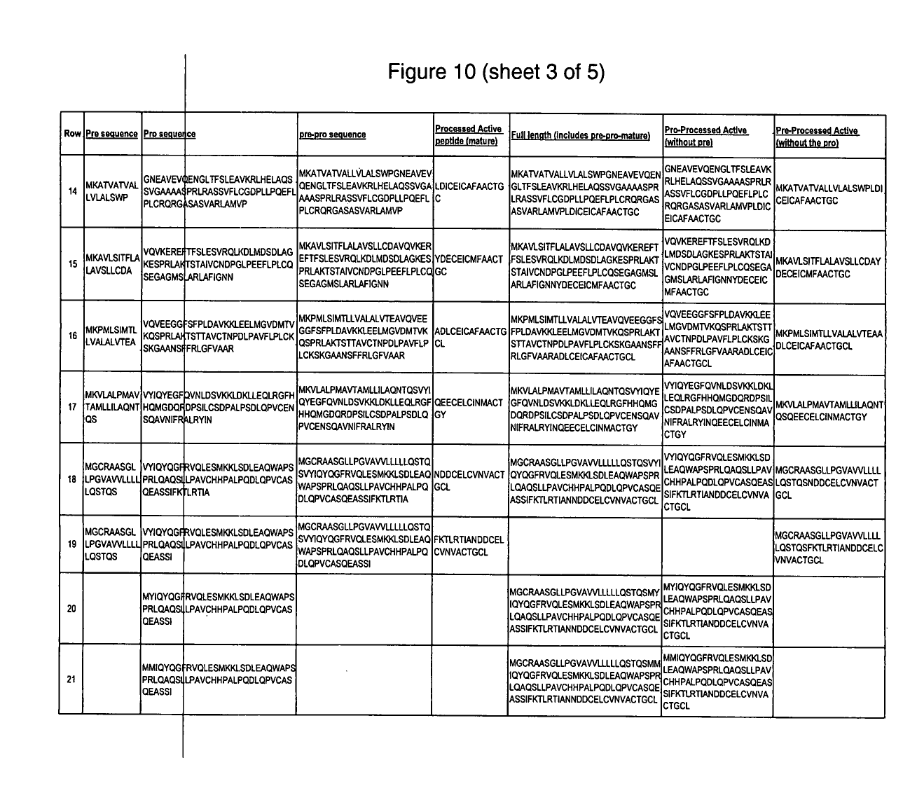

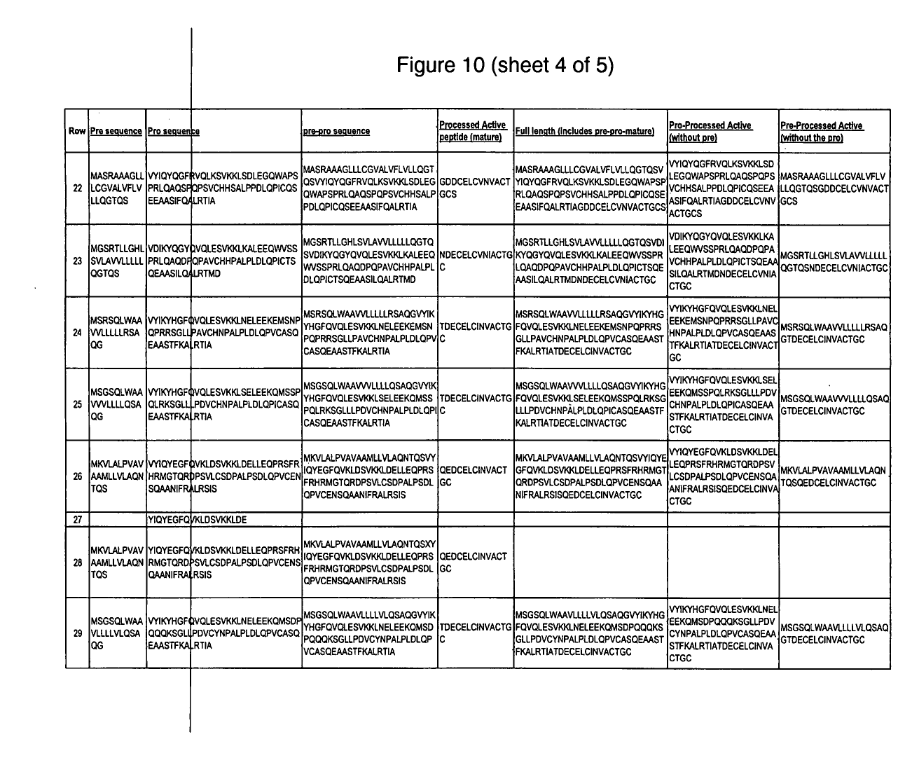

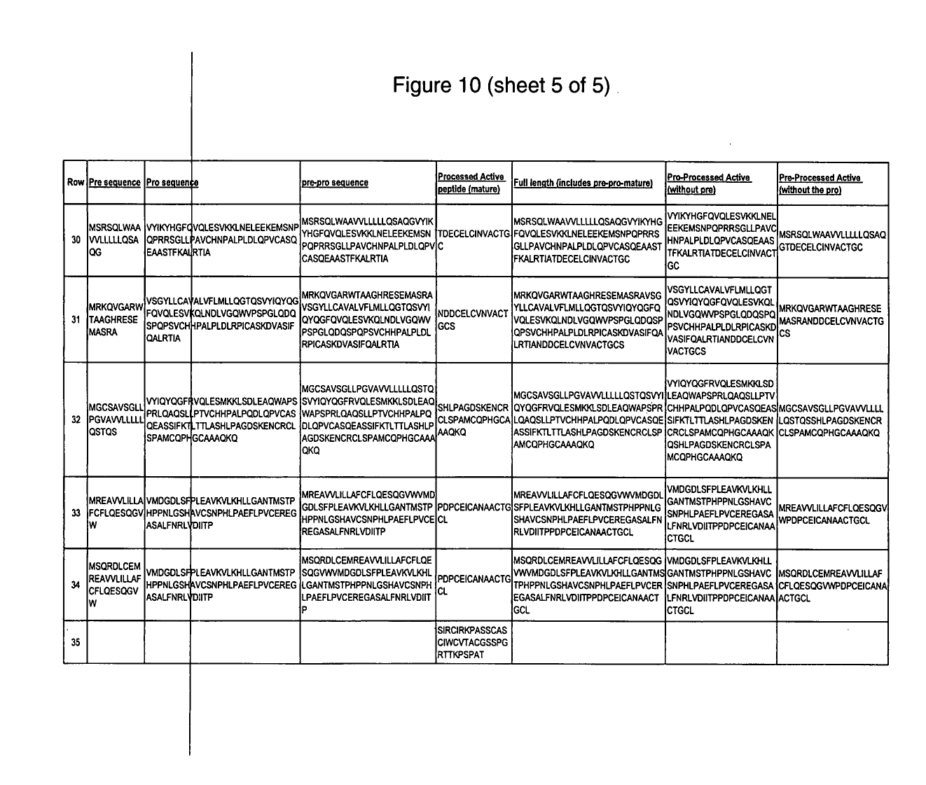

A₁′ is an amino acid sequences comprising (including, e.g., consisting of or consisting essentially of) a pre sequence depicted in FIG. 10, or an amino acid sequence comprising (including, e.g., consisting of or consisting essentially of) mnafllsalc llgawaalag gvtvqdgnfs fslesvkklk dlqepqepry gklrnfapip gepvvpilcs npnfpeelkp lckepnaqei lqrleeiaed (SEQ ID NO:17), mgcraasgll pgvavvllll lqstqsvyiq yqgfrvqles mkklsdleaq wapsprlqaq sllpavchhp alpqdlqpvc asqeassifk tlrtia (SEQ ID NO:18), lrtia (SEQ ID NO:19), mnawllsvlc llgalavlve gvtvqdgdls fplesvkqlk hlrevqeptl mshkkfalrl pkpvapelcs qsafpealrp lcekpnaeei lqrleaiaqd (SEQ ID NO:20), and msgsqlwaav llllvlqsaq gvyikyhgfq vqlesvkkln eleekqmsdp qqqksgllpd vcynpalpld lqpvcasqea astfkalrti a (SEQ ID NO:21) or a bacterial leader sequence such as: mkksilfiflsvlsfspfaqdakpvesskekitleskkcniakksnksgpesmn (SEQ ID NO:22), or is missing;

B₁′ is an amino acid sequences comprising (including, e.g., consisting of or consisting essentially of) a pro sequence depicted in FIG. 10, represented by SEQ ID NO:11, SEQ ID NO:12, SEQ ID NO:13, SEQ ID NO:14, or is missing;

C₁′ is an amino acid sequences comprising (including, e.g., consisting of or consisting essentially of) a GC-C receptor agonist polypeptide amino acid sequence depicted in FIG. 10, represented by SEQ ID NO:9, SEQ ID NO:10, SEQ ID NO:14_{A}; SEQ ID NO:15, or SEQ ID NO:16; and

wherein one or more of A₁′-B₁′-C₁′ are separated by one or more methionine residues (Z₂). For example, the peptide described herein is a polypeptide or a salt thereof comprising (including, e.g., consisting of or consisting essentially of) an amino acid sequence selected from Table 1.

TABLE 1
Guanylin and Uroguanylin Polypeptide Sequences

| Row | A₁′ | Z₂ | B₁′ | Z₂ | C₁′ |
|---|---|---|---|---|---|
| 1 | X | X or O | X | X or O | X |
| 2 | X | X | X | O | X |
| 3 | X | O | X | X | X |
| 4 | X | O | O | X | X |
| 5 | O | O | X | X | X |

Wherein X indicates that the amino acid or amino acid sequence is present and O indicates the amino acid or amino acid sequence is absent. Thus, row 4 indicates a peptide having the sequence: A₁′-Z₂-C₁′.

In some cases, described herein are purified prouroguanylin polypeptides comprising (or consisting of or consisting essentially of): (1) at least 10 contiguous amino acids of a presequence (e.g., depicted in FIG. 10, or SEQ ID NO:17, SEQ ID NO:18; SEQ ID NO:19, SEQ ID NO:20, SEQ ID NO:21, or SEQ ID NO:22); (2) at least 10 contiguous amino acids of a prosequence (e.g., depicted in FIG. 10, or SEQ ID NO: 11, SEQ ID NO:12, SEQ ID NO:13, or SEQ ID NO:14); and (3) at least 10 contiguous amino acids of a mature UGN sequence (e.g., depicted in FIG. 10, or SEQ ID NO:9 or SEQ ID NO:10), wherein the polypeptide consists includes at least: a portion of (1), (2), and at least a portion of (3), and wherein at least one of (1) and (2) or (2) and (3) are separated by one or more methionine residues.

In some cases, described herein are purified prouroguanylin polypeptides comprising (or consisting of or consisting essentially of): (1) at least 10 contiguous amino acids of a presequence (e.g., depicted in FIG. 10, or SEQ ID NO:17, SEQ ID NO:18; SEQ ID NO:19, SEQ ID NO:20, SEQ ID NO:21, or SEQ ID NO:22); (2) at least 10 contiguous amino acids of a prosequence (e.g., depicted in FIG. 10, or SEQ ID NO: 11, SEQ ID NO:12, SEQ ID NO:13, or SEQ ID NO:14); and (3) at least 10 contiguous amino acids of a mature GN sequence (e.g., depicted in FIG. 10, or SEQ ID NO:15 or SEQ ID NO:16), wherein the polypeptide consists includes at least: a portion of (1), (2), and at least a portion of (3), and wherein at least one of (1) and (2) or (2) and (3) are separated by one or more methionine residues.

In some cases, the peptides described herein are capable of binding and/or activating the GC-C receptor.

ST and Related Peptides

ST peptides, which are produced by a variety of bacteria, bind to and activate the human GC-C receptor.

In bacteria, ST peptides are derived from a preproprotein that generally has at least 70 amino acids. The pre and pro regions are cleaved as part of the secretion process, and the resulting mature peptide, which generally includes fewer than 20 amino acids, is biologically active.

Among the known bacterial ST peptides are: E. coli ST Ib (Moseley et al. 1983 Infect. Immun. 39:1167) having the mature amino acid sequence Asn Ser Ser Asn Tyr Cys Cys Glu Leu Cys Cys Asn Pro Ala Cys Thr Gly Cys Tyr (SEQ ID NO:34); E. coli ST Ia (So and McCarthy 1980 Proc. Natl. Acad. Sci. USA 77:4011) having the mature amino acid sequence Asn Thr Phe Tyr Cys Cys Glu Leu Cys Cys Asn Pro Ala Cys Ala Gly Cys Tyr (SEQ ID NO:35). E. coli ST I* (Chan and Giannella 1981 J. Biol. Chem. 256:7744) having the mature amino acid sequence Asn Thr Phe Tyr Cys Cys Glu Leu Cys Cys Tyr Pro Ala Cys Ala Gly Cys Asn (SEQ ID NO:36); C. freundii ST peptide (Guarino et al. 1989b Infect. Immun. 57:649) having the mature amino acid sequence Asn Thr Phe Tyr Cys Cys Glu Leu Cys Cys Asn Pro Ala Cys Ala Gly Cys Tyr (SEQ ID NO:35); Y. enterocolitica ST peptides, Y-ST(Y-STa), Y-STb, and Y-STc (reviewed in Huang et al. 1997 Microb. Pathog. 22:89) having the following pro-form amino acid sequences: Gln Ala Cys Asp Pro Pro Ser Pro Pro Ala Glu Val Ser Ser Asp Trp Asp Cys Cys Asp Val Cys Cys Asn Pro Ala Cys Ala Gly Cys (SEQ ID NO:27) (as well as a Ser-7 to Leu-7 variant of Y-STa (SEQ ID NO:28), (Takao et al. 1985 Eur. J. Biochem. 152:199)); Lys Ala Cys Asp Thr Gln Thr Pro Ser Pro Ser Glu Glu Asn Asp Asp Trp Cys Cys Glu Val Cys Cys Asn Pro Ala Cys Ala Gly Cys (SEQ ID NO:29); Gln Glu Thr Ala Ser Gly Gln Val Gly Asp Val Ser Ser Ser Thr Ile Ala Thr Glu Val Ser Glu Ala Glu Cys Gly Thr Gln Ser Ala Thr Thr Gln Gly Glu Asn Asp Trp Asp Trp Cys Cys Glu Leu Cys Cys Asn Pro Ala Cys Phe Gly Cys (SEQ ID NO:30), respectively; Y. kristensenii ST peptide having the mature amino acid sequence Ser Asp Trp Cys Cys Glu Val Cys Cys Asn Pro Ala Cys Ala Gly Cys (SEQ ID NO:31); V. cholerae non-01 ST peptide (Takao et al. (1985) FEBS lett. 193:250) having the mature amino acid sequence Ile Asp Cys Cys Glu Ile Cys Cys Asn Pro Ala Cys Phe Gly Cys Leu Asn (SEQ ID NO:32); and V. mimicus ST peptide (Arita et al. 1991 FEMS Microbiol. Lett. 79:105) having the mature amino acid sequence Ile Asp Cys Cys Glu Ile Cys Cys Asn Pro Ala Cys Phe Gly Cys Leu Asn (SEQ ID NO:33). Table 2 below provides sequences of all or a portion of a number of mature ST peptides and analogs thereof. Such peptides and peptides comprising these peptides are useful GCC agonists.

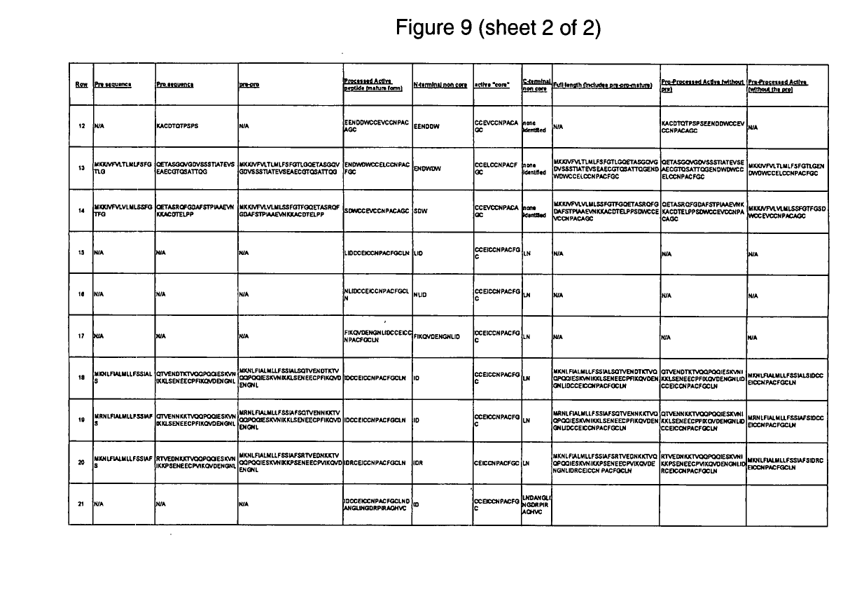

Useful polypeptides can include a naturally-occurring bacterial ST polypeptide in its mature form, as a preproprotein (includes, from amino terminus to carboxy terminus, pre sequence, pro sequence and mature peptide), as a proprotein (includes, from amino terminus to carboxy terminus, pro sequence and mature peptide) or as a prepeptide (includes, from amino terminus to carboxy terminus, pre sequence and mature peptode). FIG. 9 depicts these various bacterial ST polypeptides.

Mature ST peptides include, in some cases, an N-terminal non-core sequence and a C-terminal non-core sequence. In some cases, one or both of these non-core sequences can be removed and the peptide will still be able to bind and activate the GC-C receptor.

Various bacterial ST peptides are presented in Table 2, below.

TABLE 2
Bacterial ST

| GenBank ® | | |
|---|---|---|
| Accession | GenBank ® | |
| No. | GI No. | Sequence (SEQ ID NOs: 34-59) |
| QHECIB | 69638 | NSSNYCCELCCNPACTGCY |
| | | (SEQ ID NO: 34) |
| P01559 | 123711 | NTFYCCELCCNPACAGCY |
| | | (SEQ ID NO: 35) |
| AAA24653 | 147878 | NTFYCCELCCNPACAPCY |
| | | (SEQ ID NO: 36) |
| P01560 | 123707 | NTFYCCELCCYPACAGCN |
| | | (SEQ ID NO: 37) |
| AAA27561 | 295439 | IDCCEICCNPACFGCLN |
| | | (SEQ ID NO: 38) |
| P04429 | 123712 | IDCCEICCNPACFGCLN |
| | | (SEQ ID NO: 39) |
| S34671 | 421286 | IDCCEICCNPACF |
| | | (SEQ ID NO: 40) |
| CAA52209 | 395161 | IDCCEICCNPACFG |
| | | (SEQ ID NO: 41) |
| A54534 | 628844 | IDCCEICCNPACFGCLN |
| | | (SEQ ID NO: 42) |
| AAL02159 | 15592919 | IDRCEICCNPACFGCLN |
| | | (SEQ ID NO: 43) |
| AAA18472 | 487395 | DWDCCDVCCNPACAGC |
| | | (SEQ ID NO: 44) |
| S25659 | 282047 | DWDCCDVCCNPACAGC |
| | | (SEQ ID NO: 45) |
| P74977 | 3913874 | NDDWCCEVCCNPACAGC |
| | | (SEQ ID NO46: ) |
| BAA23656 | 2662339 | WDWCCELCCNPACFGC |
| | | (SEQ ID NO: 47) |
| P31518 | 399947 | SDWCCEVCCNPACAGC |
| | | (SEQ ID NO: 48) |
| | | QACDPPSPPAEVSSDWDCCDVCCDPACAGC |
| | | (SEQ ID NO: 49) |
| | | QACDPPSPPAEVSSDWDCCDVCCNPACAGC |
| | | (SEQ ID NO: 50) |
| | | KACDTQTPSPSEENDDTCCEVCCNPACAGC |
| | | (SEQ ID NO: 51) |
| | | QETASGQVGDVSSSTIATEVSEAECGTQSA |
| | | TTQGENDWDWCCELCCNPACFGC |
| | | (SEQ ID NO: 52) |
| | | MKKLMLAIFISVLSFPSFSQSTESLDS |
| | | SKEKITLETKKCDVVKNNSEKKSEN |
| | | MNNTFYCCELCCNPACAGCY |
| | | (SEQ ID NO: 53) |
| | | MKKSILFIFLSVLSFSPFAQDAKPVES |
| | | SKEKITLESKKCNIAKKSNKSGPESM |
| | | NSSNYCCELCCNPACTGCY |
| | | (SEQ ID NO: 54) |
| | | MKKIVFVLVLMLSSFGAFGQETVSG |
| | | QFSDALSTPITAEVYKQACDPPLPPA |
| | | EVSSDWDCCDVCCNPACAGC |
| | | (SEQ ID NO: 55) |
| | | GNLIDCCEICCNPACFGCLN |
| | | (SEQ ID NO: 56) |
| | | GNLIDRCEICCNPACFGCLN |
| | | (SEQ ID NO: 57) |
| | | PPAEVSSDWDCCDVCCNPACAGC |
| | | (SEQ ID NO: 58) |
| | | NYCCELCCNPACTGCF |
| | | (SEQ ID NO: 59) |

The immature (including pre and pro regions) form of E. coli ST-1A (ST-P) protein has the sequence: mkklmlaifisvlsfpsfsqstesldsskekitletkkcdvvknnsekksenmnntfyccelccnpacagcy (SEQ ID NO:60; see GenBank® Accession No. P01559 (gi:123711). The pre sequence extends from aa 1-19. The pro sequence extends from aa 20-54. The mature protein extends from 55-72. The immature (including pre and pro regions) form of E. coli ST-1B (ST-H) protein has the sequence: mkksilfiflsvlsfspfaqdakpvesskekitleskkcniakksnksgpesmnssnyccelccnpactgcy (SEQ ID NO:61; see GenBank® Accession No. P07965 (gi:3915589)). The immature (including pre and pro regions) form of Y. enterocolitica ST protein has the sequence: mkkivfvlvlmlssfgafgqetvsgqfsdalstpitaevykqacdpplppaevssdwdccdvccnpacagc (SEQ ID NO:62; see GenBank® Accession No. S25659 (gi:282047)).

The bacterial ST peptides have six Cys residues. These six Cys residues form three disulfide bonds in the mature and active form of the peptide. If the six Cys residues are identified, from the amino to carboxy terminus of the peptide, as A, B, C, D, E, and F, then the disulfide bonds form as follows: A-D, B-E, and C-F. The formation of these bonds is thought to be important for GC-C receptor binding.

In some cases, the ST-related peptide described herein is a polypeptide or a salt thereof comprising the amino acid sequence:
A′-B′-C′-D′-E′

wherein:

A′ is an amino acid sequence comprising (including, e.g., consisting of or consisting essentially of) a pre sequence depicted in FIG. 9 or is missing;

B′ is an amino acid sequence comprising (including, e.g., consisting of or consisting essentially of) a pro sequence depicted in FIG. 9 or is missing;

C′ is an amino acid sequence comprising (including, e.g., consisting of or consisting essentially of) an N-terminal non-core sequence depicted in FIG. 9 or is missing;

D′ is an amino acid sequence comprising (including, e.g., consisting of or consisting essentially of) a GC-C receptor agonist polypeptide amino acid sequence;

E′ is an amino acid sequence comprising (including, e.g., consisting of or consisting essentially of) a C-terminal non-core sequence depicted in FIG. 9 or is missing; and

wherein one or more of A′-B′, B′-C′, and/or C′-D are separated by one or more methionine residues (Z₂). For example, the peptide described herein is a polypeptide or a salt thereof comprising (including, e.g., consisting of or consisting essentially of) an amino acid sequence selected from Table 3.

TABLE 3
ST Polypeptide Sequences

| Row | A′ | Z₂ | B′ | Z₂ | C′ | Z₂ | D′ | E′ |
|---|---|---|---|---|---|---|---|---|
| 1 | X | X | X | X | ◯ | ◯ | X | ◯ |
| 2 | X | X | X | X | X | ◯ | X | X |
| 3 | X | X | X | X | X | X | X | X |
| 4 | X | X | X | X | X | ◯ | X | ◯ |
| 5 | X | X | X | X | ◯ | ◯ | X | X |
| 6 | ◯ | X | X | X | ◯ | ◯ | X | ◯ |
| 7 | ◯ | X | X | X | X | ◯ | X | X |
| 8 | ◯ | X | X | X | X | X | X | X |
| 9 | ◯ | X | X | X | X | ◯ | X | ◯ |
| 10 | ◯ | X | X | X | ◯ | ◯ | X | X |
| 11 | X | ◯ | X | X | ◯ | ◯ | X | ◯ |
| 12 | X | ◯ | X | X | X | ◯ | X | X |
| 13 | X | ◯ | X | X | X | X | X | X |
| 14 | X | ◯ | X | X | X | ◯ | X | ◯ |
| 15 | X | ◯ | X | X | ◯ | ◯ | X | X |
| 16 | X | X | X | ◯ | ◯ | ◯ | X | ◯ |
| 17 | X | X | X | ◯ | X | ◯ | X | X |
| 18 | X | X | X | ◯ | X | X | X | X |
| 19 | X | X | X | ◯ | X | ◯ | X | ◯ |
| 20 | X | X | X | ◯ | ◯ | ◯ | X | X |
| 21 | ◯ | ◯ | X | X | ◯ | ◯ | X | ◯ |
| 22 | ◯ | ◯ | X | X | X | ◯ | X | X |
| 23 | ◯ | ◯ | X | X | X | X | X | X |
| 24 | ◯ | ◯ | X | X | X | ◯ | X | ◯ |
| 25 | ◯ | ◯ | X | X | ◯ | ◯ | X | X |
| 26 | X | X | ◯ | ◯ | ◯ | ◯ | X | ◯ |
| 27 | X | X | ◯ | ◯ | X | ◯ | X | X |
| 28 | X | X | ◯ | ◯ | X | X | X | X |
| 29 | X | X | ◯ | ◯ | X | ◯ | X | ◯ |
| 30 | X | X | ◯ | ◯ | ◯ | ◯ | X | X |
| 31 | ◯ | ◯ | ◯ | X | ◯ | ◯ | X | ◯ |
| 32 | ◯ | ◯ | ◯ | X | X | ◯ | X | X |
| 33 | ◯ | ◯ | ◯ | X | X | X | X | X |
| 34 | ◯ | ◯ | ◯ | X | X | ◯ | X | ◯ |
| 35 | ◯ | ◯ | ◯ | X | ◯ | ◯ | X | X |

Wherein X indicates that the amino acid or amino acid sequence is present and O indicates that the amino acid or amino acid sequence is absent. Thus, row 1 in Table 3 indicates a protein comprising the sequence: A′-Z₂-B′-Z₂-D′ and row 11 indicates a protein having the sequence: A′-B′-Z₂-D′

Alternatively or in addition, the peptide described herein is a peptide or a salt thereof comprising (including, e.g., consisting of or consisting essentially of) the amino acid sequence:
A′-B′-Z₂-D′;
A′-Z₂-D′; and/or
B′-Z₂-D′;

wherein in each amino acid sequence C′ and/or E′ are present or are missing and Z₂ consists of one or more methionine residues.

In some cases, each occurrence of Z₂ represents one methionine residue.

In some cases: D′ comprises the amino acid sequence Xaa₁ Xaa₂ Xaa₃ Xaa₄ Xaa₅ Cys₆ Cys₇ Xaa₈ Xaa₉ Cys₁₀ Cys₁₁ Xaa₁₂ Xaa₁₃ Xaa₁₄ Cys₁₅ Xaa₁₆ Xaa₁₇ Cys₁₈ Xaa₁₉ Xaa₂₀ Xaa₂₁ (SEQ ID NO: 63) wherein:
Xaa₈, Xaa₉, Xaa₁₂, Xaa₁₃, Xaa₁₄, Xaa₁₆ and Xaa₁₇ are independently any amino acid;
Xaa₁, Xaa₂, Xaa₃, Xaa₄ and Xaa₅ are independently any amino acid or one or more amino acids within the sequence Xaa₁ Xaa₂ Xaa₃ Xaa₄ Xaa₅ is missing;
Xaa₁₉, Xaa₂₀ and Xaa₂₁ are independently any amino acid or one or more amino acids within the sequence Xaa₁₉ Xaa₂₀ Xaa₂₁ is missing; or the sequence Xaa₂₀ Xaa₂₁ is missing and Xaa₁₉ is any amino acid, or the sequence Xaa₂₀ Xaa₂₁ is missing and Xaa₁₉ is Tyr;

the sequence Xaa₁ Xaa₂ Xaa₃ Xaa₄ Xaa₅ is missing;

Xaa₁₉ Xaa₂₀ Xaa₂₁ is missing or the sequence Xaa₂₀ Xaa₂₁ is missing and;
Xaa₁₉ is Tyr;
Xaa₈ is Glu;
Xaa₉ is Leu, Tyr, Phe or Trp;
Xaa₁₂ is Asn;
Xaa₁₃ is Pro;
Xaa₁₄ is Ala;
Xaa₁₆ is Thr; and
Xaa₁₇ is Gly.

In some cases of SEQ ID NO:63, Xaa₁, Xaa₂, Xaa₃, Xaa₄, Xaa₅, Xaa₈, Xaa₉, Xaa₁₂, Xaa₁₃, Xaa₁₄, Xaa₁₆, Xaa₁₇, Xaa₁₉, Xaa₂₀ and Xaa₂₁ are any amino acid, e.g., Ala, Arg, Asn, Asp, Cys, Gln, Glu, Gly, His, Ile, Leu, Lys, Met, Phe, Pro, Ser, Thr, Trp, Tyr, and Val. In some cases, of SEQ ID NO:63, the sequence Xaa₁ Xaa₂ Xaa₃ Xaa₄ Xaa₅ is missing. In some cases of SEQ ID NO:63, the sequence Xaa₁₉ Xaa₂₀ Xaa₂₁ is missing or the sequence Xaa₂₀ Xaa₂₁ is missing and Xaa₁₉ is Tyr. In some cases of SEQ ID NO:63, Xaa₁ Xaa₂ Xaa₃ Xaa₄ Xaa₅ is missing, the sequence Xaa₂₀ Xaa₂₁ is missing and Xaa₁₉ is Tyr.

In some cases of SEQ ID NO: 63: Xaa₁ Xaa₂ Xaa₃ Xaa₄ Xaa₅ is Asn Ser Ser Asn Tyr or is missing or Xaa₁ Xaa₂ Xaa₃ Xaa₄ is missing.

In some cases of SEQ ID NO: 63: Xaa₈, Xaa₉, Xaa₁₂, Xaa₁₄, Xaa₁₆, Xaa₁₇, and Xaa₁₉ can be any amino acid. In some cases of SEQ ID NO:63, Xaa₈, Xaa₉, Xaa₁₂, Xaa₁₄, Xaa₁₆, Xaa₁₇, and Xaa₁₉ can be any natural or non-natural amino acid or amino acid analog.

In some cases of SEQ ID NO: 63: Xaa₅ is Asn, Trp, Tyr, Asp, or Phe. In other cases, Xaa₅ can also be Thr or Ile. In other cases, Xaa₅ is Tyr, Asp or Trp. In certain cases Xaa₅ is Asn, Trp, Tyr, Asp, Ile, Thr or Phe. In some cases, Xaa₅ is Asn.

In some cases of SEQ ID NO: 63: Xaa₈ is Glu, Asp, Gln, Gly or Pro. In other cases Xaa₈ is Glu. In other cases Xaa₈ is Glu or Asp. In others it is Asn, Glu, or Asp. In others it is Glu, His, Lys, Gln, Asn, or Asp. In others it is Glu, His, Gln, Asn, or Asp. In others it is Glu, Asn, His, Gln, Lys, Asp or Ser. In still others it is Pro. In certain cases it is any natural or non-natural amino acid or amino acid analog.

In some cases of SEQ ID NO: 63: Xaa₉ is Leu, Ile, Val, Ala, Lys, Arg, Trp, Tyr or Phe. In some cases Xaa₉ is Leu, Ile, Val, Lys, Arg, Trp, Tyr or Phe. In others it is Leu, Ile, Val, Trp, Tyr or Phe. In others it is Leu, Ile or Val. In others it is Trp, Tyr or Phe. In others it is Leu, Ile, Lys, Arg, Trp, Tyr, or Phe. In others it is Leu, Val, Ile, or Met. In others it is Leu or Phe. In others it is Leu, Phe, or Tyr. In others it is Tyr, Phe or His. In others it is Phe, His, Trp, or Tyr. In certain cases, Xaa₉ is not Leu. In others it is Tyr. In other cases it is any natural or non-natural aromatic amino acid or amino acid analog. In certain cases it is any natural or non-natural amino acid or amino acid analog.

In certain cases of SEQ ID NO: 63: Xaa₁₂ is Asn, Tyr, Asp or Ala. In others it is Asn. In others it is Asn, Met, Arg, Lys, His, or Gln. In others it is Asn, Lys, His, or Gln. In others it is Asn, Asp, Glu or Gln. In others it is Asn, Thr, Ser, Arg, Lys, Gln, or His. In others it is Asn, Ser, or His. In certain cases it is any natural or non-natural amino acid or amino acid analog.

In certain cases of SEQ ID NO: 63: Xaa₁₃ is Ala, Pro or Gly. In others it is Pro or Gly. In others it is Pro and in still others it is Gly.

In certain cases of SEQ ID NO: 63: Xaa₁₄ is Ala, Leu, Ser, Gly, Val, Glu, Gln, Ile, Leu, Thr, Lys, Arg, or Asp. In others it is Ala or Gly. In others it is Val or Ala. In others it is Ala or Thr. In others it is Ala. In others it is Val, Gln, Asn, Glu, Asp, Thr, or Ala. In others it is Gly, Cys or Ser. In still others it is Thr. In certain cases it is any natural or non-natural amino acid or amino acid analog.

In certain cases of SEQ ID NO: 63: Xaa₁₆ is Thr, Ala, Asn, Lys, Arg, Trp, Gly or Val. In others it is Thr, Ala, Asn, Lys, Arg or Trp. In others it is Thr, Ala, Lys, Arg or Trp. In certain cases it is Thr, Ala or Trp. In others it is Thr. In certain cases it is Trp, Tyr or Phe. In certain cases it is Thr or Ala. In certain cases it is Val. In certain cases it is Gly. In others it is Thr, Ser, Met or Val. In others it is Val, Ala, or Thr. In others it is Ile, Val, Lys, Asn, Glu, Asp, or Thr. In certain cases it is any natural or non-natural amino acid or amino acid analog. In certain cases it is any natural or non-natural non-aromatic amino acid or amino acid analog.

In certain cases of SEQ ID NO: 63: Xaa₁₇ is Gly, Pro or Ala. In certain cases it is Gly. In certain cases it is Ala. In others it is Gly or Ala. In others it is Gly, Asn, Ser or Ala. In others it is Asn, Glu, Asp, Thr, Ala, Ser, or Gly. In others it is Asp, Ala, Ser, or Gly. In certain cases it is any natural or non-natural amino acid or amino acid analog.

In certain cases of SEQ ID NO: 63: Xaa₁₉ is Trp, Tyr, Phe, Asn, Ile, Val, His, Leu, or Arg. In certain cases it is Tip, Tyr, Asn or Leu. In certain cases it is Tip, Tyr or Phe. In others it is Tyr, Phe or His. In others it is Tyr or Trp. In others it is Tyr. In certain cases it is Leu, Ile or Val. In certain cases it is His. In certain cases it is Tip, Tyr, Phe, Asn, Ile, Val, His or Leu. In certain cases it is Trp, Tyr, Phe or Leu. In certain cases it is Tyr or Leu. In certain cases it is Lys or Arg. In certain cases it is any amino acid other than Pro, Arg, Lys, Asp or Glu. In certain cases it is any amino acid other than Pro. In certain cases it is any natural or non-natural amino acid or amino acid analog. In certain cases it is missing.

In certain cases of SEQ ID NO: 63: Xaa₂₀ is Asp or Asn. In certain cases Xaa₂₀ Xaa₂₁ is AspPhe or is missing or Xaa₂₀ is Asn or Glu and Xaa₂₁ is missing or Xaa₁₉ Xaa₂₀ Xaa₂₁ is missing.

In some cases, the peptides disclosed herein do not include the sequence of E. coli ST peptide. In other cases, the peptide does not include the sequence of any of the peptides in Table 2, above.

In certain cases D′ comprises the amino acid sequence SEQ ID NO: 64.

(SEQ ID NO: 64)
Xaa₁ Xaa₂ Xaa₃ Xaa₄ Xaa₅ Cys₆ Cys₇ Xaa₈ Xaa₉ Cys₁₀
Cys₁₁ Asn₁₂ Pro₁₃ Ala₁₄ Cys₁₅ Xaa₁₆ Gly₁₇ Cys₁₈
Xaa₁₉ Xaa₂₀ Xaa₂₁.

wherein Xaa₁ Xaa₂ Xaa₃ Xaa₄ Xaa₅ is Asn Ser Ser Asn Tyr or is missing or Xaa₁ Xaa₂ Xaa₃ Xaa₄ is missing and Xaa₅ is Asn;

Xaa₈ is Glu or Asp;

Xaa₉ is Leu, Ile, Val, Trp, Tyr or Phe;

Xaa₁₆ is Thr, Ala, Trp;

Xaa₁₉ is Trp, Tyr, Phe or Leu or is missing; and Xaa₂₀ Xaa₂₁ is AspPhe.

In various cases of SEQ ID NO:64: Xaa₉ is Leu, Ile or Val and Xaa₁₆ is Trp, Tyr or Phe; Xaa₉ is Trp, Tyr or Phe, and Xaa₁₆ is Thr or Ala; Xaa₁₉ is Trp, Tyr, Phe and Xaa₂₀ Xaa₂₁ is AspPhe; and Xaa₁ Xaa₂ Xaa₃ Xaa₄ is missing and Xaa₅ is Asn; the peptide comprises fewer than 50, 40, 30 or 25 amino acids; or fewer than five amino acids precede Cys₆.

In certain cases the peptide includes a peptide comprising or consisting of the amino acid sequence of SEQ ID NO:64 wherein Xaa₉ is any amino acid: wherein Xaa₉ is any amino acid other than Leu; wherein Xaa₉ is selected from Phe, Trp and Tyr; wherein Xaa₉ is selected from any other natural or non-natural aromatic amino acid; wherein Xaa₉ is Tyr; wherein Xaa₉ is Phe; wherein Xaa₉ is Trp; wherein Xaa₁ Xaa₂ Xaa₃ Xaa₄ Xaa₅ is Asn Ser Ser Asn Tyr; wherein Xaa₁, Xaa₂, Xaa₃, Xaa₄, and Xaa₅ are missing; wherein Xaa₁, Xaa₂, Xaa₃ and Xaa₄ are missing; wherein Xaa₁, Xaa₂ and Xaa₃ are missing; wherein Xaa₁ and Xaa₂ are missing; wherein Xaa₁ is missing; wherein Xaa₂₀ Xaa₂₁ is AspPhe or is missing or Xaa₂₀ is Asn or Glu and Xaa₂₁ is missing or Xaa₁₉ Xaa₂₀ Xaa₂₁ is missing; wherein Xaa₁ Xaa₂ Xaa₃ Xaa₄ Xaa₅ and Tyr Xaa₂₀ Xaa₂₁ are missing.

In the case of a peptide comprising the sequence of SEQ ID NO:63 wherein: Xaa₁ Xaa₂ Xaa₃ Xaa₄ Xaa₅ is missing and/or the sequence Xaa₁₉ Xaa₂₀ Xaa₂₁ is missing, the peptide can still contain additional carboxy terminal or amino terminal amino acids or both. In the case of peptides missing one or more terminal amino acids such as Xaa₁ or Xaa₂₁, the peptide can still contain additional carboxy terminal or amino terminal amino acids or both.

In certain cases, the peptide includes disulfide bonds between Cys₆ and Cys₁₁, between Cys₇ and Cys₁₅ and between Cys₁₀ and Cys₁₆. In other cases, the peptide is a reduced peptide having no disulfide bonds. In still other cases the peptide has one or two disulfide bonds chosen from: a disulfide bond between Cys₆ and Cys₁₁, a disulfide bond between Cys₇ and Cys₁₅ and a disulfide bond between Cys₁₀ and Cys₁₆.

In some cases, D′ comprises an amino acid sequence selected from (SEQ ID NOs:65-104, respectively, as shown below):

(SEQ ID NO: 65)
Cys Cys Glu Ala Cys Cys Asn Pro Ala Cys Thr Gly
Cys
(SEQ ID NO: 66)
Cys Cys Glu Arg Cys Cys Asn Pro Ala Cys Thr Gly
Cys
(SEQ ID NO: 67)
Cys Cys Glu Asn Cys Cys Asn Pro Ala Cys Thr Gly
Cys
(SEQ ID NO: 68)
Cys Cys Glu Asp Cys Cys Asn Pro Ala Cys Thr Gly
Cys
(SEQ ID NO: 69)
Cys Cys Glu Cys Cys Cys Asn Pro Ala Cys Thr Gly
Cys
(SEQ ID NO: 70)
Cys Cys Glu Gln Cys Cys Asn Pro Ala Cys Thr Gly
Cys
(SEQ ID NO: 71)
Cys Cys Glu Glu Cys Cys Asn Pro Ala Cys Thr Gly
Cys
(SEQ ID NO: 72)
Cys Cys Glu Gly Cys Cys Asn Pro Ala Cys Thr Gly
Cys
(SEQ ID NO: 73)
Cys Cys Glu His Cys Cys Asn Pro Ala Cys Thr Gly
Cys
(SEQ ID NO: 74)
Cys Cys Glu Ile Cys Cys Asn Pro Ala Cys Thr Gly
Cys
(SEQ ID NO: 75)
Cys Cys Glu Lys Cys Cys Asn Pro Ala Cys Thr Gly
Cys
(SEQ ID NO: 76)
Cys Cys Glu Met Cys Cys Asn Pro Ala Cys Thr Gly
Cys
(SEQ ID NO: 77)
Cys Cys Glu Phe Cys Cys Asn Pro Ala Cys Thr Gly
Cys
(SEQ ID NO: 78)
Cys Cys Glu Pro Cys Cys Asn Pro Ala Cys Thr Gly
Cys
(SEQ ID NO: 79)
Cys Cys Glu Ser Cys Cys Asn Pro Ala Cys Thr Gly
Cys
(SEQ ID NO: 80)
Cys Cys Glu Thr Cys Cys Asn Pro Ala Cys Thr Gly
Cys
(SEQ ID NO: 81)
Cys Cys Glu Trp Cys Cys Asn Pro Ala Cys Thr Gly
Cys
(SEQ ID NO: 82)
Cys Cys Glu Val Cys Cys Asn Pro Ala Cys Thr Gly
Cys
(SEQ ID NO: 83)
Cys Cys Glu Tyr Cys Cys Asn Pro Ala Cys Thr Gly
Cys
(SEQ ID NO: 84)
Cys Cys Glu Leu Cys Cys Asn Pro Ala Cys Thr Gly
Cys
(SEQ ID NO: 85)
Cys Cys Glu Ala Cys Cys Asn Pro Ala Cys Thr Gly
Cys Tyr
(SEQ ID NO: 86)
Cys Cys Glu Arg Cys Cys Asn Pro Ala Cys Thr Gly
Cys Tyr
(SEQ ID NO: 87)
Cys Cys Glu Asn Cys Cys Asn Pro Ala Cys Thr Gly
Cys Tyr
(SEQ ID NO: 88)
Cys Cys Glu Asp Cys Cys Asn Pro Ala Cys Thr Gly
Cys Tyr
(SEQ ID NO: 89)
Cys Cys Glu Cys Cys Cys Asn Pro Ala Cys Thr Gly
Cys Tyr
(SEQ ID NO: 90)
Cys Cys Glu Gln Cys Cys Asn Pro Ala Cys Thr Gly
Cys Tyr
(SEQ ID NO: 91)
Cys Cys Glu Glu Cys Cys Asn Pro Ala Cys Thr Gly
Cys Tyr
(SEQ ID NO: 92)
Cys Cys Glu Gly Cys Cys Asn Pro Ala Cys Thr Gly
Cys Tyr
(SEQ ID NO: 93)
Cys Cys Glu His Cys Cys Asn Pro Ala Cys Thr Gly
Cys Tyr
(SEQ ID NO: 94)
Cys Cys Glu Ile Cys Cys Asn Pro Ala Cys Thr Gly
Cys Tyr
(SEQ ID NO: 95)
Cys Cys Glu Lys Cys Cys Asn Pro Ala Cys Thr Gly
Cys Tyr
(SEQ ID NO: 96)
Cys Cys Glu Met Cys Cys Asn Pro Ala Cys Thr Gly
Cys Tyr
(SEQ ID NO: 97)
Cys Cys Glu Phe Cys Cys Asn Pro Ala Cys Thr Gly
Cys Tyr
(SEQ ID NO: 98)
Cys Cys Glu Pro Cys Cys Asn Pro Ala Cys Thr Gly
Cys Tyr
(SEQ ID NO: 99)
Cys Cys Glu Ser Cys Cys Asn Pro Ala Cys Thr Gly
Cys Tyr
(SEQ ID NO: 100)
Cys Cys Glu Thr Cys Cys Asn Pro Ala Cys Thr Gly
Cys Tyr
(SEQ ID NO: 101)
Cys Cys Glu Trp Cys Cys Asn Pro Ala Cys Thr Gly
Cys Tyr
(SEQ ID NO: 102)
Cys Cys Glu Val Cys Cys Ash Pro Ala Cys Thr Gly
Cys Tyr
(SEQ ID NO: 103)
Cys Cys Glu Tyr Cys Cys Asn Pro Ala Cys Thr Gly
Cys Tyr;
and
(SEQ ID NO: 104)
Cys Cys Glu Leu Cys Cys Asn Pro Ala Cys Thr Gly
Cys Tyr.

In various cases of the aforementioned polypeptides: D′ comprises (consists of or consists essentially of) an amino acid sequence selected from SEQ ID NOs:105-112, shown below, respectively):

| | |
|---|---|
| CCELCCNPACTGCY; | (SEQ ID NO: 105) |
| CCEYCCNPACTGCY; | (SEQ ID NO: 106) |
| CCEFCCNPACTGCY; | (SEQ ID NO: 107) |
| CCEWCCNPACTGCY; | (SEQ ID NO: 108) |
| CCELCCNPACTGC; | (SEQ ID NO: 109) |
| CCEYCCNPACTGC; | (SEQ ID NO: 110) |
| CCEFCCNPACTGC; | (SEQ ID NO: 111) |
| and | |
| CCEWCCNPACTGC. | (SEQ ID NO: 112) |

In some cases: D′ comprises an amino acid sequence selected from SEQ ID NOs: 113-806, respectively, as shown below:

| | |
|---|---|
| Gln Ser Ser Asn Tyr Cys Cys Glu Tyr Cys Cys Asn Pro Ala Cys Thr Gly Cys Tyr | (SEQ ID NO: 113) |
| Asn Thr Ser Asn Tyr Cys Cys Glu Tyr Cys Cys Asn Pro Ala Cys Thr Gly Cys Tyr | (SEQ ID NO: 114) |
| Asn Leu Ser Asn Tyr Cys Cys Glu Tyr Cys Cys Asn Pro Ala Cys Thr Gly Cys Tyr | (SEQ ID NO: 115) |
| Asn Ile Ser Asn Tyr Cys Cys Glu Tyr Cys Cys Asn Pro Ala Cys Thr Gly Cys Tyr | (SEQ ID NO: 116) |
| Asn Ser Ser Gln Tyr Cys Cys Glu Tyr Cys Cys Asn Pro Ala Cys Thr Gly Cys Tyr | (SEQ ID NO: 117) |
| Ser Ser Asn Tyr Cys Cys Glu Tyr Cys Cys Asn Pro Ala Cys Thr Gly Cys Tyr | (SEQ ID NO: 118) |
| Gln Ser Ser Gln Tyr Cys Cys Glu Tyr Cys Cys Asn Pro Ala Cys Thr Gly Cys Tyr | (SEQ ID NO: 119) |
| Ser Ser Gln Tyr Cys Cys Glu Tyr Cys Cys Asn Pro Ala Cys Thr Gly Cys Tyr. | (SEQ ID NO: 120) |
| Asn Ser Ser Asn Tyr Cys Cys Glu Ala Cys Cys Asn Pro Ala Cys Thr Gly Cys Tyr | (SEQ ID NO: 121) |
| Asn Ser Ser Asn Tyr Cys Cys Glu Arg Cys Cys Asn Pro Ala Cys Thr Gly Cys Tyr | (SEQ ID NO: 122) |
| Asn Ser Ser Asn Tyr Cys Cys Glu Asn Cys Cys Asn Pro Ala Cys Thr Gly Cys Tyr | (SEQ ID NO: 123) |
| Asn Ser Ser Asn Tyr Cys Cys Glu Asp Cys Cys Asn Pro Ala Cys Thr Gly Cys Tyr | (SEQ ID NO: 124) |
| Asn Ser Ser Asn Tyr Cys Cys Glu Cys Cys Cys Asn Pro Ala Cys Thr Gly Cys Tyr | (SEQ ID NO: 125) |
| Asn Ser Ser Asn Tyr Cys Cys Glu Gln Cys Cys Asn Pro Ala Cys Thr Gly Cys Tyr | (SEQ ID NO: 126) |
| Asn Ser Ser Asn Tyr Cys Cys Glu Glu Cys Cys Asn Pro Ala Cys Thr Gly Cys Tyr | (SEQ ID NO: 127) |
| Asn Ser Ser Asn Tyr Cys Cys Glu Gly Cys Cys Asn Pro Ala Cys Thr Gly Cys Tyr | (SEQ ID NO: 128) |
| Asn Ser Ser Asn Tyr Cys Cys Glu His Cys Cys Asn Pro Ala Cys Thr Gly Cys Tyr | (SEQ ID NO: 129) |
| Asn Ser Ser Asn Tyr Cys Cys Glu Ile Cys Cys Asn Pro Ala Cys Thr Gly Cys Tyr | (SEQ ID NO: 130) |
| Asn Ser Ser Asn Tyr Cys Cys Glu Lys Cys Cys Asn Pro Ala Cys Thr Gly Cys Tyr | (SEQ ID NO: 131) |
| Asn Ser Ser Asn Tyr Cys Cys Glu Met Cys Cys Asn Pro Ala Cys Thr Gly Cys Tyr | (SEQ ID NO: 132) |
| Asn Ser Ser Asn Tyr Cys Cys Glu Phe Cys Cys Asn Pro Ala Cys Thr Gly Cys Tyr | (SEQ ID NO: 133) |
| Asn Ser Ser Asn Tyr Cys Cys Glu Pro Cys Cys Asn Pro Ala Cys Thr Gly Cys Tyr | (SEQ ID NO: 134) |
| Asn Ser Ser Asn Tyr Cys Cys Glu Ser Cys Cys Asn Pro Ala Cys Thr Gly Cys Tyr | (SEQ ID NO: 135) |
| Asn Ser Ser Asn Tyr Cys Cys Glu Thr Cys Cys Asn Pro Ala Cys Thr Gly Cys Tyr | (SEQ ID NO: 136) |
| Asn Ser Ser Asn Tyr Cys Cys Glu Trp Cys Cys Asn Pro Ala Cys Thr Gly Cys Tyr | (SEQ ID NO: 137) |
| Asn Ser Ser Asn Tyr Cys Cys Glu Val Cys Cys Asn Pro Ala Cys Thr Gly Cys Tyr | (SEQ ID NO: 138) |
| Cys Cys Glu Ala Cys Cys Asn Pro Ala Cys Thr Gly Cys Tyr | (SEQ ID NO: 139) |
| Cys Cys Glu Arg Cys Cys Asn Pro Ala Cys Thr Gly Cys Tyr | (SEQ ID NO: 140) |
| Cys Cys Glu Asn Cys Cys Asn Pro Ala Cys Thr Gly Cys Tyr | (SEQ ID NO: 141) |
| Cys Cys Glu Asp Cys Cys Asn Pro Ala Cys Thr Gly Cys Tyr | (SEQ ID NO: 142) |
| Cys Cys Glu Cys Cys Cys Asn Pro Ala Cys Thr Gly Cys Tyr | (SEQ ID NO: 143) |
| Cys Cys Glu Gln Cys Cys Asn Pro Ala Cys Thr Gly Cys Tyr | (SEQ ID NO: 144) |
| Cys Cys Glu Glu Cys Cys Asn Pro Ala Cys Thr Gly Cys Tyr | (SEQ ID NO: 145) |
| Cys Cys Glu Gly Cys Cys Asn Pro Ala Cys Thr Gly Cys Tyr | (SEQ ID NO: 146) |
| Cys Cys Glu His Cys Cys Asn Pro Ala Cys Thr Gly Cys Tyr | (SEQ ID NO: 147) |
| Cys Cys Glu Ile Cys Cys Asn Pro Ala Cys Thr Gly Cys Tyr | (SEQ ID NO: 148) |
| Cys Cys Glu Lys Cys Cys Asn Pro Ala Cys Thr Gly Cys Tyr | (SEQ ID NO: 149) |
| Cys Cys Glu Met Cys Cys Asn Pro Ala Cys Thr Gly Cys Tyr | (SEQ ID NO: 150) |
| Cys Cys Glu Phe Cys Cys Asn Pro Ala Cys Thr Gly Cys Tyr | (SEQ ID NO: 151) |
| Cys Cys Glu Pro Cys Cys Asn Pro Ala Cys Thr Gly Cys Tyr | (SEQ ID NO: 152 |
| Cys Cys Glu Ser Cys Cys Asn Pro Ala Cys Thr Gly Cys Tyr | (SEQ ID NO: 153) |
| Cys Cys Glu Thr Cys Cys Asn Pro Ala Cys Thr Gly Cys Tyr | (SEQ ID NO: 154) |
| Cys Cys Glu Trp Cys Cys Asn Pro Ala Cys Thr Gly Cys Tyr | (SEQ ID NO: 155) |
| Cys Cys Glu Val Cys Cys Asn Pro Ala Cys Thr Gly Cys Tyr | (SEQ ID NO: 156) |
| Cys Cys Glu Tyr Cys Cys Asn Pro Ala Cys Thr Gly Cys | (SEQ ID NO: 157) |
| Cys Cys Glu Ala Cys Cys Asn Pro Ala Cys Thr Gly Cys | (SEQ ID NO: 158) |
| Cys Cys Glu Arg Cys Cys Asn Pro Ala Cys Thr Gly Cys | (SEQ ID NO: 159) |
| Cys Cys Glu Asn Cys Cys Asn Pro Ala Cys Thr Gly Cys | (SEQ ID NO: 160) |
| Cys Cys Glu Asp Cys Cys Asn Pro Ala Cys Thr Gly Cys | (SEQ ID NO: 161) |
| Cys Cys Glu Cys Cys Cys Asn Pro Ala Cys Thr Gly Cys | (SEQ ID NO: 162) |
| Cys Cys Glu Gln Cys Cys Asn Pro Ala Cys Thr Gly Cys | (SEQ ID NO: 163) |
| Cys Cys Glu Glu Cys Cys Asn Pro Ala Cys Thr Gly Cys | (SEQ ID NO: 164) |
| Cys Cys Glu Gly Cys Cys Asn Pro Ala Cys Thr Gly Cys | (SEQ ID NO: 165) |
| Cys Cys Glu His Cys Cys Asn Pro Ala Cys Thr Gly Cys | (SEQ ID NO: 166) |
| Cys Cys Glu Ile Cys Cys Asn Pro Ala Cys Thr Gly Cys | (SEQ ID NO: 167) |
| Cys Cys Glu Lys Cys Cys Asn Pro Ala Cys Thr Gly Cys | (SEQ ID NO: 168) |
| Cys Cys Glu Met Cys Cys Asn Pro Ala Cys Thr Gly Cys | (SEQ ID NO: 169) |
| Cys Cys Glu Phe Cys Cys Asn Pro Ala Cys Thr Gly Cys | (SEQ ID NO: 170) |
| Cys Cys Glu Pro Cys Cys Asn Pro Ala Cys Thr Gly Cys | (SEQ ID NO: 171) |
| Cys Cys Glu Ser Cys Cys Asn Pro Ala Cys Thr Gly Cys | (SEQ ID NO: 172) |
| Cys Cys Glu Thr Cys Cys Asn Pro Ala Cys Thr Gly Cys; | (SEQ ID NO: 173) |
| Cys Cys Glu Trp Cys Cys Asn Pro Ala Cys Thr Gly Cys | (SEQ ID NO: 174) |
| Cys Cys Glu Val Cys Cys Asn Pro Ala Cys Thr Gly Cys. | (SEQ ID NO: 175) |
| Cys Cys Glu Leu Cys Cys Ala Pro Ala Cys Thr Gly Cys Tyr | (SEQ ID NO: 176) |
| Cys Cys Glu Leu Cys Cys Val Pro Ala Cys Thr Gly Cys Tyr | (SEQ ID NO: 177) |
| Cys Cys Glu Leu Cys Cys Leu Pro Ala Cys Thr Gly Cys Tyr | (SEQ ID NO: 178) |
| Cys Cys Glu Leu Cys Cys Ile Pro Ala Cys Thr Gly Cys Tyr | (SEQ ID NO: 179) |
| Cys Cys Glu Leu Cys Cys Pro Pro Ala Cys Thr Gly Cys Tyr | (SEQ ID NO: 180) |
| Cys Cys Glu Leu Cys Cys Met Pro Ala Cys Thr Gly Cys Tyr | (SEQ ID NO: 181) |
| Cys Cys Glu Leu Cys Cys Phe Pro Ala Cys Thr Gly Cys Tyr | (SEQ ID NO: 182) |
| Cys Cys Glu Leu Cys Cys Trp Pro Ala Cys Thr Gly Cys Tyr | (SEQ ID NO: 183) |
| Cys Cys Glu Leu Cys Cys Gly Pro Ala Cys Thr Gly Cys Tyr | (SEQ ID NO: 184) |
| Cys Cys Glu Leu Cys Cys Ser Pro Ala Cys Thr Gly Cys Tyr | (SEQ ID NO: 185) |
| Cys Cys Glu Leu Cys Cys Thr Pro Ala Cys Thr Gly Cys Tyr | (SEQ ID NO: 186) |
| Cys Cys Glu Leu Cys Cys Cys Pro Ala Cys Thr Gly Cys Tyr | (SEQ ID NO: 187) |
| Cys Cys Glu Leu Cys Cys Gln Pro Ala Cys Thr Gly Cys Tyr | (SEQ ID NO: 188) |
| Cys Cys Glu Leu Cys Cys Tyr Pro Ala Cys Thr Gly Cys Tyr | (SEQ ID NO: 189) |
| Cys Cys Glu Leu Cys Cys Asp Pro Ala Cys Thr Gly Cys Tyr | (SEQ ID NO: 190) |
| Cys Cys Glu Leu Cys Cys Glu Pro Ala Cys Thr Gly Cys Tyr | (SEQ ID NO: 200) |
| Cys Cys Glu Leu Cys Cys Lys Pro Ala Cys Thr Gly Cys Tyr | (SEQ ID NO: 201) |
| Cys Cys Glu Leu Cys Cys Arg Pro Ala Cys Thr Gly Cys Tyr | (SEQ ID NO: 202) |
| Cys Cys Glu Leu Cys Cys His Pro Ala Cys Thr Gly Cys Tyr | (SEQ ID NO: 203) |
| Cys Cys Glu Tyr Cys Cys Ala Pro Ala Cys Thr Gly Cys Tyr | (SEQ ID NO: 204) |
| Cys Cys Glu Tyr Cys Cys Val Pro Ala Cys Thr Gly Cys Tyr | (SEQ ID NO: 205) |
| Cys Cys Glu Tyr Cys Cys Leu Pro Ala Cys Thr Gly Cys Tyr | (SEQ ID NO: 206) |
| Cys Cys Glu Tyr Cys Cys Ile Pro Ala Cys Thr Gly Cys Tyr | (SEQ ID NO: 207) |
| Cys Cys Glu Tyr Cys Cys Pro Pro Ala Cys Thr Gly Cys Tyr | (SEQ ID NO: 208) |
| Cys Cys Glu Tyr Cys Cys Met Pro Ala Cys Thr Gly Cys Tyr | (SEQ ID NO: 209) |
| Cys Cys Glu Tyr Cys Cys Phe Pro Ala Cys Thr Gly Cys Tyr | (SEQ ID NO: 300) |
| Cys Cys Glu Tyr Cys Cys Trp Pro Ala Cys Thr Gly Cys Tyr | (SEQ ID NO: 301) |
| Cys Cys Glu Tyr Cys Cys Gly Pro Ala Cys Thr Gly Cys Tyr | (SEQ ID NO: 302) |
| Cys Cys Glu Tyr Cys Cys Ser Pro Ala Cys Thr Gly Cys Tyr | (SEQ ID NO: 303) |
| Cys Cys Glu Tyr Cys Cys Thr Pro Ala Cys Thr Gly Cys Tyr | (SEQ ID NO: 304) |
| Cys Cys Glu Tyr Cys Cys Cys Pro Ala Cys Thr Gly Cys Tyr | (SEQ ID NO: 305) |
| Cys Cys Glu Tyr Cys Cys Gln Pro Ala Cys Thr Gly Cys Tyr | (SEQ ID NO: 306) |
| Cys Cys Glu Tyr Cys Cys Tyr Pro Ala Cys Thr Gly Cys Tyr | (SEQ ID NO: 307) |
| Cys Cys Glu Tyr Cys Cys Asp Pro Ala Cys Thr Gly Cys Tyr | (SEQ ID NO: 308) |
| Cys Cys Glu Tyr Cys Cys Glu Pro Ala Cys Thr Gly Cys Tyr | (SEQ ID NO: 309) |
| Cys Cys Glu Tyr Cys Cys Lys Pro Ala Cys Thr Gly Cys Tyr | (SEQ ID NO: 310) |
| Cys Cys Glu Tyr Cys Cys Arg Pro Ala Cys Thr Gly Cys Tyr | (SEQ ID NO: 311) |
| Cys Cys Glu Tyr Cys Cys His Pro Ala Cys Thr Gly Cys Tyr | (SEQ ID NO: 312) |
| Cys Cys Glu Leu Cys Cys Ala Pro Ala Cys Thr Gly Cys | (SEQ ID NO: 313) |
| Cys Cys Glu Leu Cys Cys Val Pro Ala Cys Thr Gly Cys | (SEQ ID NO: 314) |
| Cys Cys Glu Leu Cys Cys Leu Pro Ala Cys Thr Gly Cys | (SEQ ID NO: 315) |
| Cys Cys Glu Leu Cys Cys Ile Pro Ala Cys Thr Gly Cys | (SEQ ID NO: 316) |
| Cys Cys Glu Leu Cys Cys Pro Pro Ala Cys Thr Gly Cys | (SEQ ID NO: 317) |
| Cys Cys Glu Leu Cys Cys Met Pro Ala Cys Thr Gly Cys | (SEQ ID NO: 318) |
| Cys Cys Glu Leu Cys Cys Phe Pro Ala Cys Thr Gly Cys | (SEQ ID NO: 319) |
| Cys Cys Glu Leu Cys Cys Trp Pro Ala Cys Thr Gly Cys | (SEQ ID NO: 320) |
| Cys Cys Glu Leu Cys Cys Gly Pro Ala Cys Thr Gly Cys | (SEQ ID NO: 321) |
| Cys Cys Glu Leu Cys Cys Ser Pro Ala Cys Thr Gly Cys | (SEQ ID NO: 322) |
| Cys Cys Glu Leu Cys Cys Thr Pro Ala Cys Thr Gly Cys | (SEQ ID NO: 323) |
| Cys Cys Glu Leu Cys Cys Cys Pro Ala Cys Thr Gly Cys | (SEQ ID NO: 324) |
| Cys Cys Glu Leu Cys Cys Gin Pro Ala Cys Thr Gly Cys | (SEQ ID NO: 325) |
| Cys Cys Glu Leu Cys Cys Tyr Pro Ala Cys Thr Gly Cys | (SEQ ID NO: 326) |
| Cys Cys Glu Leu Cys Cys Asp Pro Ala Cys Thr Gly Cys | (SEQ ID NO: 327) |
| Cys Cys Glu Leu Cys Cys Glu Pro Ala Cys Thr Gly Cys | (SEQ ID NO: 328) |
| Cys Cys Glu Leu Cys Cys Lys Pro Ala Cys Thr Gly Cys | (SEQ ID NO: 329) |
| Cys Cys Glu Leu Cys Cys Arg Pro Ala Cys Thr Gly Cys | (SEQ ID NO: 330) |
| Cys Cys Glu Leu Cys Cys His Pro Ala Cys Thr Gly Cys | (SEQ ID NO: 331) |
| Cys Cys Glu Tyr Cys Cys Ala Pro Ala Cys Thr Gly Cys | (SEQ ID NO: 332) |
| Cys Cys Glu Tyr Cys Cys Val Pro Ala Cys Thr Gly Cys | (SEQ ID NO: 333) |
| Cys Cys Glu Tyr Cys Cys Leu Pro Ala Cys Thr Gly Cys | (SEQ ID NO: 334) |
| Cys Cys Glu Tyr Cys Cys Ile Pro Ala Cys Thr Gly Cys | (SEQ ID NO: 335) |
| Cys Cys Glu Tyr Cys Cys Pro Pro Ala Cys Thr Gly Cys | (SEQ ID NO: 336) |
| Cys Cys Glu Tyr Cys Cys Met Pro Ala Cys Thr Gly Cys | (SEQ ID NO: 337) |
| Cys Cys Glu Tyr Cys Cys Phe Pro Ala Cys Thr Gly Cys | (SEQ ID NO: 338) |
| Cys Cys Glu Tyr Cys Cys Trp Pro Ala Cys Thr Gly Cys | (SEQ ID NO: 339) |
| Cys Cys Glu Tyr Cys Cys Gly Pro Ala Cys Thr Gly Cys | (SEQ ID NO: 340) |
| Cys Cys Glu Tyr Cys Cys Ser Pro Ala Cys Thr Gly Cys | (SEQ ID NO: 341) |
| Cys Cys Glu Tyr Cys Cys Thr Pro Ala Cys Thr Gly Cys | (SEQ ID NO: 342) |
| Cys Cys Glu Tyr Cys Cys Cys Pro Ala Cys Thr Gly Cys | (SEQ ID NO: 343) |
| Cys Cys Glu Tyr Cys Cys Gln Pro Ala Cys Thr Gly Cys | (SEQ ID NO: 344) |
| Cys Cys Glu Tyr Cys Cys Tyr Pro Ala Cys Thr Gly Cys | (SEQ ID NO: 345) |
| Cys Cys Glu Tyr Cys Cys Asp Pro Ala Cys Thr Gly Cys | (SEQ ID NO: 346) |
| Cys Cys Glu Tyr Cys Cys Glu Pro Ala Cys Thr Gly Cys | (SEQ ID NO: 347) |
| Cys Cys Glu Tyr Cys Cys Lys Pro Ala Cys Thr Gly Cys | (SEQ ID NO: 348) |
| Cys Cys Glu Tyr Cys Cys Arg Pro Ala Cys Thr Gly Cys | (SEQ ID NO: 349) |
| Cys Cys Glu Tyr Cys Cys His Pro Ala Cys Thr Gly Cys | (SEQ ID NO: 350) |
| Cys Cys Glu Leu Cys Cys Asn Pro Thr Cys Thr Gly Cys Tyr | (SEQ ID NO: 351) |
| Cys Cys Glu Tyr Cys Cys Asn Pro Thr Cys Thr Gly Cys Tyr | (SEQ ID NO: 352) |
| Cys Cys Glu Leu Cys Cys Asn Pro Thr Cys Thr Gly Cys | (SEQ ID NO: 353) |
| Cys Cys Glu Tyr Cys Cys Asn Pro Thr Cys Thr Gly Cys | (SEQ ID NO: 354) |
| Cys Cys Glu Phe Cys Cys Asn Pro Thr Cys Thr Gly Cys Tyr | (SEQ ID NO: 355) |
| Cys Cys Glu Phe Cys Cys Asn Pro Thr Cys Thr Gly Cys | (SEQ ID NO: 356) |
| Cys Cys Glu Trp Cys Cys Asn Pro Thr Cys Thr Gly Cys Tyr | (SEQ ID NO: 357) |
| Cys Cys Glu Trp Cys Cys Asn Pro Thr Cys Thr Gly Cys | (SEQ ID NO: 358) |
| Cys Cys Glu Leu Cys Cys Asn Gly Ala Cys Thr Gly Cys Tyr | (SEQ ID NO: 359) |
| Cys Cys Glu Tyr Cys Cys Asn Gly Ala Cys Thr Gly Cys Tyr | (SEQ ID NO: 360) |
| Cys Cys Glu Leu Cys Cys Asn Gly Ala Cys Thr Gly Cys | (SEQ ID NO: 361) |
| Cys Cys Glu Tyr Cys Cys Asn Gly Ala Cys Thr Gly Cys | (SEQ ID NO: 362) |
| Cys Cys Glu Phe Cys Cys Asn Gly Ala Cys Thr Gly Cys Tyr | (SEQ ID NO: 363) |
| Cys Cys Glu Phe Cys Cys Asn Gly Ala Cys Thr Gly Cys | (SEQ ID NO: 364) |
| Cys Cys Glu Trp Cys Cys Asn Gly Ala Cys Thr Gly Cys Tyr | (SEQ ID NO: 365) |
| Cys Cys Glu Trp Cys Cys Asn Gly Ala Cys Thr Gly Cys | (SEQ ID NO: 366) |
| Cys Cys Glu Leu Cys Cys Asn Pro Ala Cys Val Gly Cys Tyr | (SEQ ID NO: 367) |
| Cys Cys Glu Tyr Cys Cys Asn Pro Ala Cys Val Gly Cys Tyr | (SEQ ID NO: 368) |
| Cys Cys Glu Leu Cys Cys Asn Pro Ala Cys Val Gly Cys | (SEQ ID NO: 369) |
| Cys Cys Glu Tyr Cys Cys Asn Pro Ala Cys Val Gly Cys | (SEQ ID NO: 370) |
| Cys Cys Glu Phe Cys Cys Asn Pro Ala Cys Val Gly Cys Tyr | (SEQ ID NO: 371) |
| Cys Cys Glu Phe Cys Cys Asn Pro Ala Cys Val Gly Cys | (SEQ ID NO: 372) |
| Cys Cys Glu Trp Cys Cys Asn Pro Ala Cys Val Gly Cys Tyr | (SEQ ID NO: 373) |
| Cys Cys Glu Trp Cys Cys Asn Pro Ala Cys Val Gly Cys | (SEQ ID NO: 374) |
| Cys Cys Glu Leu Cys Cys Asn Pro Ala Cys Gly Gly Cys Tyr | (SEQ ID NO: 375) |
| Cys Cys Glu Tyr Cys Cys Asn Pro Ala Cys Gly Gly Cys Tyr | (SEQ ID NO: 376) |
| Cys Cys Glu Leu Cys Cys Asn Pro Ala Cys Gly Gly Cys | (SEQ ID NO: 377) |
| Cys Cys Glu Tyr Cys Cys Asn Pro Ala Cys Gly Gly Cys | (SEQ ID NO: 378) |
| Cys Cys Glu Phe Cys Cys Asn Pro Ala Cys Gly Gly Cys Tyr | (SEQ ID NO: 379) |
| Cys Cys Glu Phe Cys Cys Asn Pro Ala Cys Gly Gly Cys | (SEQ ID NO: 380) |
| Cys Cys Glu Trp Cys Cys Asn Pro Ala Cys Gly Gly Cys Tyr | (SEQ ID NO: 381) |
| Cys Cys Glu Trp Cys Cys Asn Pro Ala Cys Gly Gly Cys | (SEQ ID NO: 382) |
| Cys Cys Glu Leu Cys Cys Asn Pro Ala Cys Thr Ala Cys Tyr | (SEQ ID NO: 383) |
| Cys Cys Glu Tyr Cys Cys Asn Pro Ala Cys Thr Ala Cys Tyr | (SEQ ID NO: 384) |
| Cys Cys Glu Leu Cys Cys Asn Pro Ala Cys Thr Ala Cys | (SEQ ID NO: 385) |
| Cys Cys Glu Tyr Cys Cys Asn Pro Ala Cys Thr Ala Cys | (SEQ ID NO: 386) |
| Cys Cys Glu Trp Cys Cys Asn Pro Ala Cys Thr Ala Cys Tyr | (SEQ ID NO: 387) |
| Cys Cys Glu Trp Cys Cys Asn Pro Ala Cys Thr Ala Cys | (SEQ ID NO: 388) |
| Cys Cys Glu Phe Cys Cys Asn Pro Ala Cys Thr Ala Cys Tyr | (SEQ ID NO: 389) |
| Cys Cys Glu Phe Cys Cys Asn Pro Ala Cys Thr Ala Cys | (SEQ ID NO: 390) |
| Cys Cys Glu Leu Cys Cys Asn Pro Ala Cys Thr Gly Cys Ala | (SEQ ID NO: 391) |
| Cys Cys Glu Leu Cys Cys Asn Pro Ala Cys Thr Gly Cys Val | (SEQ ID NO: 392) |
| Cys Cys Glu Leu Cys Cys Asn Pro Ala Cys Thr Gly Cys Leu | (SEQ ID NO: 393) |
| Cys Cys Glu Leu Cys Cys Asn Pro Ala Cys Thr Gly Cys Ile | (SEQ ID NO: 394) |
| Cys Cys Glu Leu Cys Cys Asn Pro Ala Cys Thr Gly Cys Pro | (SEQ ID NO: 395) |
| Cys Cys Glu Leu Cys Cys Asn Pro Ala Cys Thr Gly Cys Met | (SEQ ID NO: 396) |
| Cys Cys Glu Leu Cys Cys Asn Pro Ala Cys Thr Gly Cys Phe | (SEQ ID NO: 397) |
| Cys Cys Glu Leu Cys Cys Asn Pro Ala Cys Thr Gly Cys Trp | (SEQ ID NO: 398) |
| Cys Cys Glu Leu Cys Cys Asn Pro Ala Cys Thr Gly Cys Gly | (SEQ ID NO: 399) |
| Cys Cys Glu Leu Cys Cys Asn Pro Ala Cys Thr Gly Cys Ser | (SEQ ID NO: 400) |
| Cys Cys Glu Leu Cys Cys Asn Pro Ala Cys Thr Gly Cys Thr | (SEQ ID NO: 401) |
| Cys Cys Glu Leu Cys Cys Asn Pro Ala Cys Thr Gly Cys Cys | (SEQ ID NO: 402) |
| Cys Cys Glu Leu Cys Cys Asn Pro Ala Cys Thr Gly Cys Asn | (SEQ ID NO: 403) |
| Cys Cys Glu Leu Cys Cys Asn Pro Ala Cys Thr Gly Cys Gln | (SEQ ID NO: 404) |
| Cys Cys Glu Leu Cys Cys Asn Pro Ala Cys Thr Gly Cys Asp | (SEQ ID NO: 405) |
| Cys Cys Glu Leu Cys Cys Asn Pro Ala Cys Thr Gly Cys Glu | (SEQ ID NO: 406) |
| Cys Cys Glu Leu Cys Cys Asn Pro Ala Cys Thr Gly Cys Lys | (SEQ ID NO: 407) |
| Cys Cys Glu Leu Cys Cys Asn Pro Ala Cys Thr Gly Cys Arg | (SEQ ID NO: 408) |
| Cys Cys Glu Leu Cys Cys Asn Pro Ala Cys Thr Gly Cys His | (SEQ ID NO: 409) |
| Cys Cys Glu Tyr Cys Cys Asn Pro Ala Cys Thr Gly Cys Ala | (SEQ ID NO: 410) |
| Cys Cys Glu Tyr Cys Cys Asn Pro Ala Cys Thr Gly Cys Val | (SEQ ID NO: 411) |
| Cys Cys Glu Tyr Cys Cys Asn Pro Ala Cys Thr Gly Cys Leu | (SEQ ID NO: 412) |
| Cys Cys Glu Tyr Cys Cys Asn Pro Ala Cys Thr Gly Cys Ile | (SEQ ID NO: 413) |
| Cys Cys Glu Tyr Cys Cys Asn Pro Ala Cys Thr Gly Cys Pro | (SEQ ID NO: 414) |
| Cys Cys Glu Tyr Cys Cys Asn Pro Ala Cys Thr Gly Cys Met | (SEQ ID NO: 415) |
| Cys Cys Glu Tyr Cys Cys Asn Pro Ala Cys Thr Gly Cys Phe | (SEQ ID NO: 416) |
| Cys Cys Glu Tyr Cys Cys Asn Pro Ala Cys Thr Gly Cys Trp | (SEQ ID NO: 417) |
| Cys Cys Glu Tyr Cys Cys Asn Pro Ala Cys Thr Gly Cys Gly | (SEQ ID NO: 418) |
| Cys Cys Glu Tyr Cys Cys Asn Pro Ala Cys Thr Gly Cys Ser | (SEQ ID NO: 419) |
| Cys Cys Glu Tyr Cys Cys Asn Pro Ala Cys Thr Gly Cys Thr | (SEQ ID NO: 420) |
| Cys Cys Glu Tyr Cys Cys Asn Pro Ala Cys Thr Gly Cys Cys | (SEQ ID NO: 421) |
| Cys Cys Glu Tyr Cys Cys Asn Pro Ala Cys Thr Gly Cys Asn | (SEQ ID NO: 422) |
| Cys Cys Glu Tyr Cys Cys Asn Pro Ala Cys Thr Gly Cys Gln | (SEQ ID NO: 423) |
| Cys Cys Glu Tyr Cys Cys Asn Pro Ala Cys Thr Gly Cys Asp | (SEQ ID NO: 424) |
| Cys Cys Glu Tyr Cys Cys Asn Pro Ala Cys Thr Gly Cys Glu | (SEQ ID NO: 425) |
| Cys Cys Glu Tyr Cys Cys Asn Pro Ala Cys Thr Gly Cys Lys | (SEQ ID NO: 426) |
| Cys Cys Glu Tyr Cys Cys Asn Pro Ala Cys Thr Gly Cys Arg | (SEQ ID NO: 427) |
| Cys Cys Glu Tyr Cys Cys Asn Pro Ala Cys Thr Gly Cys His | (SEQ ID NO: 428) |
| Cys Cys Ala Leu Cys Cys Asn Pro Ala Cys Thr Gly Cys Tyr | (SEQ ID NO: 429) |
| Cys Cys Val Leu Cys Cys Asn Pro Ala Cys Thr Gly Cys Tyr | (SEQ ID NO: 430) |
| Cys Cys Leu Leu Cys Cys Asn Pro Ala Cys Thr Gly Cys Tyr | (SEQ ID NO: 431) |
| Cys Cys Ile Leu Cys Cys Asn Pro Ala Cys Thr Gly Cys Tyr | (SEQ ID NO: 432) |
| Cys Cys Met Leu Cys Cys Asn Pro Ala Cys Thr Gly Cys Tyr | (SEQ ID NO: 433) |
| Cys Cys Phe Leu Cys Cys Asn Pro Ala Cys Thr Gly Cys Tyr | (SEQ ID NO: 434) |
| Cys Cys Trp Leu Cys Cys Asn Pro Ala Cys Thr Gly Cys Tyr | (SEQ ID NO: 435) |
| Cys Cys Gly Leu Cys Cys Asn Pro Ala Cys Thr Gly Cys Tyr | (SEQ ID NO: 436) |
| Cys Cys Ser Leu Cys Cys Asn Pro Ala Cys Thr Gly Cys Tyr | (SEQ ID NO: 437) |
| Cys Cys Thr Leu Cys Cys Asn Pro Ala Cys Thr Gly Cys Tyr | (SEQ ID NO: 438) |
| Cys Cys Cys Leu Cys Cys Asn Pro Ala Cys Thr Gly Cys Tyr | (SEQ ID NO: 439) |
| Cys Cys Asn Leu Cys Cys Asn Pro Ala Cys Thr Gly Cys Tyr | (SEQ ID NO: 440) |
| Cys Cys Gln Leu Cys Cys Asn Pro Ala Cys Thr Gly Cys Tyr | (SEQ ID NO: 441) |
| Cys Cys Tyr Leu Cys Cys Asn Pro Ala Cys Thr Gly Cys Tyr | (SEQ ID NO: 442) |
| Cys Cys Asp Leu Cys Cys Asn Pro Ala Cys Thr Gly Cys Tyr | (SEQ ID NO: 443) |
| Cys Cys Lys Leu Cys Cys Asn Pro Ala Cys Thr Gly Cys Tyr | (SEQ ID NO: 444) |
| Cys Cys Arg Leu Cys Cys Asn Pro Ala Cys Thr Gly Cys Tyr | (SEQ ID NO: 445) |
| Cys Cys His Leu Cys Cys Asn Pro Ala Cys Thr Gly Cys Tyr | (SEQ ID NO: 446) |
| Cys Cys Ala Leu Cys Cys Asn Pro Ala Cys Thr Gly Cys | (SEQ ID NO: 447) |
| Cys Cys Val Leu Cys Cys Asn Pro Ala Cys Thr Gly Cys | (SEQ ID NO: 448) |
| Cys Cys Leu Leu Cys Cys Asn Pro Ala Cys Thr Gly Cys | (SEQ ID NO: 449) |
| Cys Cys Ile Leu Cys Cys Asn Pro Ala Cys Thr Gly Cys | (SEQ ID NO: 450) |
| Cys Cys Met Leu Cys Cys Asn Pro Ala Cys Thr Gly Cys | (SEQ ID NO: 451) |
| Cys Cys Phe Leu Cys Cys Asn Pro Ala Cys Thr Gly Cys | (SEQ ID NO: 452) |
| Cys Cys Trp Leu Cys Cys Asn Pro Ala Cys Thr Gly Cys | (SEQ ID NO: 453) |
| Cys Cys Gly Leu Cys Cys Asn Pro Ala Cys Thr Gly Cys | (SEQ ID NO: 454) |
| Cys Cys Ser Leu Cys Cys Asn Pro Ala Cys Thr Gly Cys | (SEQ ID NO: 455) |
| Cys Cys Thr Leu Cys Cys Asn Pro Ala Cys Thr Gly Cys | (SEQ ID NO: 456) |
| Cys Cys Cys Leu Cys Cys Asn Pro Ala Cys Thr Gly Cys | (SEQ ID NO: 457) |
| Cys Cys Asn Leu Cys Cys Asn Pro Ala Cys Thr Gly Cys | (SEQ ID NO: 458) |
| Cys Cys Gln Leu Cys Cys Asn Pro Ala Cys Thr Gly Cys | (SEQ ID NO: 459) |
| Cys Cys Tyr Leu Cys Cys Asn Pro Ala Cys Thr Gly Cys | (SEQ ID NO: 460) |
| Cys Cys Asp Leu Cys Cys Asn Pro Ala Cys Thr Gly Cys | (SEQ ID NO: 461) |
| Cys Cys Lys Leu Cys Cys Asn Pro Ala Cys Thr Gly Cys | (SEQ ID NO: 462) |
| Cys Cys Arg Leu Cys Cys Asn Pro Ala Cys Thr Gly Cys | (SEQ ID NO: 463) |
| Cys Cys His Leu Cys Cys Asn Pro Ala Cys Thr Gly Cys | (SEQ ID NO: 464) |
| Cys Cys Ala Tyr Cys Cys Asn Pro Ala Cys Thr Gly Cys Tyr | (SEQ ID NO: 465) |
| Cys Cys Val Tyr Cys Cys Asn Pro Ala Cys Thr Gly Cys Tyr | (SEQ ID NO: 466) |
| Cys Cys Leu Tyr Cys Cys Asn Pro Ala Cys Thr Gly Cys Tyr | (SEQ ID NO: 467) |
| Cys Cys Ile Tyr Cys Cys Asn Pro Ala Cys Thr Gly Cys Tyr | (SEQ ID NO: 468) |
| Cys Cys Met Tyr Cys Cys Asn Pro Ala Cys Thr Gly Cys Tyr | (SEQ ID NO: 469) |
| Cys Cys Phe Tyr Cys Cys Asn Pro Ala Cys Thr Gly Cys Tyr | (SEQ ID NO: 470) |
| Cys Cys Trp Tyr Cys Cys Asn Pro Ala Cys Thr Gly Cys Tyr | (SEQ ID NO: 471) |
| Cys Cys Gly Tyr Cys Cys Asn Pro Ala Cys Thr Gly Cys Tyr | (SEQ ID NO: 472) |
| Cys Cys Ser Tyr Cys Cys Asn Pro Ala Cys Thr Gly Cys Tyr | (SEQ ID NO: 473) |
| Cys Cys Thr Tyr Cys Cys Asn Pro Ala Cys Thr Gly Cys Tyr | (SEQ ID NO: 474) |
| Cys Cys Cys Tyr Cys Cys Asn Pro Ala Cys Thr Gly Cys Tyr | (SEQ ID NO: 475) |
| Cys Cys Asn Tyr Cys Cys Asn Pro Ala Cys Thr Gly Cys Tyr | (SEQ ID NO: 476) |
| Cys Cys Gln Tyr Cys Cys Asn Pro Ala Cys Thr Gly Cys Tyr | (SEQ ID NO: 477) |
| Cys Cys Tyr Tyr Cys Cys Asn Pro Ala Cys Thr Gly Cys Tyr | (SEQ ID NO: 478) |
| Cys Cys Asp Tyr Cys Cys Asn Pro Ala Cys Thr Gly Cys Tyr | (SEQ ID NO: 479) |
| Cys Cys Lys Tyr Cys Cys Asn Pro Ala Cys Thr Gly Cys Tyr | (SEQ ID NO: 480) |
| Cys Cys Arg Tyr Cys Cys Asn Pro Ala Cys Thr Gly Cys Tyr | (SEQ ID NO: 481) |
| Cys Cys His Tyr Cys Cys Asn Pro Ala Cys Thr Gly Cys Tyr | (SEQ ID NO: 482) |
| Cys Cys Ala Tyr Cys Cys Asn Pro Ala Cys Thr Gly Cys | (SEQ ID NO: 483) |
| Cys Cys Val Tyr Cys Cys Asn Pro Ala Cys Thr Gly Cys | (SEQ ID NO: 484) |
| Cys Cys Leu Tyr Cys Cys Asn Pro Ala Cys Thr Gly Cys | (SEQ ID NO: 485) |
| Cys Cys Ile Tyr Cys Cys Asn Pro Ala Cys Thr Gly Cys | (SEQ ID NO: 486) |
| Cys Cys Met Tyr Cys Cys Asn Pro Ala Cys Thr Gly Cys | (SEQ ID NO: 487) |
| Cys Cys Phe Tyr Cys Cys Asn Pro Ala Cys Thr Gly Cys | (SEQ ID NO: 488) |
| Cys Cys Trp Tyr Cys Cys Asn Pro Ala Cys Thr Gly Cys | (SEQ ID NO: 489) |
| Cys Cys Gly Tyr Cys Cys Asn Pro Ala Cys Thr Gly Cys | (SEQ ID NO: 490) |
| Cys Cys Ser Tyr Cys Cys Asn Pro Ala Cys Thr Gly Cys | (SEQ ID NO: 491) |
| Cys Cys Thr Tyr Cys Cys Asn Pro Ala Cys Thr Gly Cys | (SEQ ID NO: 492) |
| Cys Cys Cys Tyr Cys Cys Asn Pro Ala Cys Thr Gly Cys | (SEQ ID NO: 493) |
| Cys Cys Asn Tyr Cys Cys Asn Pro Ala Cys Thr Gly Cys | (SEQ ID NO: 494) |
| Cys Cys Gln Tyr Cys Cys Asn Pro Ala Cys Thr Gly Cys | (SEQ ID NO: 495) |
| Cys Cys Tyr Tyr Cys Cys Asn Pro Ala Cys Thr Gly Cys | (SEQ ID NO: 496) |
| Cys Cys Asp Tyr Cys Cys Asn Pro Ala Cys Thr Gly Cys | (SEQ ID NO: 497) |
| Cys Cys Lys Tyr Cys Cys Asn Pro Ala Cys Thr Gly Cys | (SEQ ID NO: 498) |
| Cys Cys Arg Tyr Cys Cys Asn Pro Ala Cys Thr Gly Cys | (SEQ ID NO: 499) |
| Cys Cys His Tyr Cys Cys Asn Pro Ala Cys Thr Gly Cys | (SEQ ID NO: 500) |
| Cys Cys Glu Phe Cys Cys Ala Pro Ala Cys Thr Gly Cys Tyr | (SEQ ID NO: 501) |
| Cys Cys Glu Phe Cys Cys Val Pro Ala Cys Thr Gly Cys Tyr | (SEQ ID NO: 502) |
| Cys Cys Glu Phe Cys Cys Leu Pro Ala Cys Thr Gly Cys Tyr | (SEQ ID NO: 503) |
| Cys Cys Glu Phe Cys Cys Ile Pro Ala Cys Thr Gly Cys Tyr | (SEQ ID NO: 504) |
| Cys Cys Glu Phe Cys Cys Pro Pro Ala Cys Thr Gly Cys Tyr | (SEQ ID NO: 505) |
| Cys Cys Glu Phe Cys Cys Met Pro Ala Cys Thr Gly Cys Tyr | (SEQ ID NO: 506) |
| Cys Cys Glu Phe Cys Cys Phe Pro Ala Cys Thr Gly Cys Tyr | (SEQ ID NO: 507) |
| Cys Cys Glu Phe Cys Cys Trp Pro Ala Cys Thr Gly Cys Tyr | (SEQ ID NO: 508) |
| Cys Cys Glu Phe Cys Cys Gly Pro Ala Cys Thr Gly Cys Tyr | (SEQ ID NO: 509) |
| Cys Cys Glu Phe Cys Cys Ser Pro Ala Cys Thr Gly Cys Tyr | (SEQ ID NO: 510) |
| Cys Cys Glu Phe Cys Cys Thr Pro Ala Cys Thr Gly Cys Tyr | (SEQ ID NO: 511) |
| Cys Cys Glu Phe Cys Cys Cys Pro Ala Cys Thr Gly Cys Tyr | (SEQ ID NO: 512) |
| Cys Cys Glu Phe Cys Cys Gln Pro Ala Cys Thr Gly Cys Tyr | (SEQ ID NO: 513) |
| Cys Cys Glu Phe Cys Cys Tyr Pro Ala Cys Thr Gly Cys Tyr | (SEQ ID NO: 514) |
| Cys Cys Glu Phe Cys Cys Asp Pro Ala Cys Thr Gly Cys Tyr | (SEQ ID NO: 515) |
| Cys Cys Glu Phe Cys Cys Glu Pro Ala Cys Thr Gly Cys Tyr | (SEQ ID NO: 516) |
| Cys Cys Glu Phe Cys Cys Lys Pro Ala Cys Thr Gly Cys Tyr | (SEQ ID NO: 517) |
| Cys Cys Glu Phe Cys Cys Arg Pro Ala Cys Thr Gly Cys Tyr | (SEQ ID NO: 518) |
| Cys Cys Glu Phe Cys Cys His Pro Ala Cys Thr Gly Cys Tyr | (SEQ ID NO: 519) |
| Cys Cys Glu Phe Cys Cys Ala Pro Ala Cys Thr Gly Cys | (SEQ ID NO: 520) |
| Cys Cys Glu Phe Cys Cys Val Pro Ala Cys Thr Gly Cys | (SEQ ID NO: 521) |
| Cys Cys Glu Phe Cys Cys Leu Pro Ala Cys Thr Gly Cys | (SEQ ID NO: 522) |
| Cys Cys Glu Phe Cys Cys Ile Pro Ala Cys Thr Gly Cys | (SEQ ID NO: 523) |
| Cys Cys Glu Phe Cys Cys Pro Pro Ala Cys Thr Gly Cys | (SEQ ID NO: 524) |
| Cys Cys Glu Phe Cys Cys Met Pro Ala Cys Thr Gly Cys | (SEQ ID NO: 525) |
| Cys Cys Glu Phe Cys Cys Phe Pro Ala Cys Thr Gly Cys | (SEQ ID NO: 526) |
| Cys Cys Glu Phe Cys Cys Trp Pro Ala Cys Thr Gly Cys | (SEQ ID NO: 527) |
| Cys Cys Glu Phe Cys Cys Gly Pro Ala Cys Thr Gly Cys | (SEQ ID NO: 528) |
| Cys Cys Glu Phe Cys Cys Ser Pro Ala Cys Thr Gly Cys | (SEQ ID NO: 529) |
| Cys Cys Glu Phe Cys Cys Thr Pro Ala Cys Thr Gly Cys | (SEQ ID NO: 530) |
| Cys Cys Glu Phe Cys Cys Cys Pro Ala Cys Thr Gly Cys | (SEQ ID NO: 531) |
| Cys Cys Glu Phe Cys Cys Gln Pro Ala Cys Thr Gly Cys | (SEQ ID NO: 532) |
| Cys Cys Glu Phe Cys Cys Tyr Pro Ala Cys Thr Gly Cys | (SEQ ID NO: 533) |
| Cys Cys Glu Phe Cys Cys Asp Pro Ala Cys Thr Gly Cys | (SEQ ID NO: 534) |
| Cys Cys Glu Phe Cys Cys Glu Pro Ala Cys Thr Gly Cys | (SEQ ID NO: 535) |
| Cys Cys Glu Phe Cys Cys Lys Pro Ala Cys Thr Gly Cys | (SEQ ID NO: 536) |
| Cys Cys Glu Phe Cys Cys Arg Pro Ala Cys Thr Gly Cys | (SEQ ID NO: 537) |
| Cys Cys Glu Phe Cys Cys His Pro Ala Cys Thr Gly Cys | (SEQ ID NO: 538) |
| Cys Cys Glu Trp Cys Cys Ala Pro Ala Cys Thr Gly Cys Tyr | (SEQ ID NO: 539) |
| Cys Cys Glu Trp Cys Cys Val Pro Ala Cys Thr Gly Cys Tyr | (SEQ ID NO: 540) |
| Cys Cys Glu Trp Cys Cys Leu Pro Ala Cys Thr Gly Cys Tyr | (SEQ ID NO: 541) |
| Cys Cys Glu Trp Cys Cys Ile Pro Ala Cys Thr Gly Cys Tyr | (SEQ ID NO: 542) |
| Cys Cys Glu Trp Cys Cys Pro Pro Ala Cys Thr Gly Cys Tyr | (SEQ ID NO: 543) |
| Cys Cys Glu Trp Cys Cys Met Pro Ala Cys Thr Gly Cys Tyr | (SEQ ID NO: 544) |
| Cys Cys Glu Trp Cys Cys Phe Pro Ala Cys Thr Gly Cys Tyr | (SEQ ID NO: 545) |
| Cys Cys Glu Trp Cys Cys Trp Pro Ala Cys Thr Gly Cys Tyr | (SEQ ID NO: 546) |
| Cys Cys Glu Trp Cys Cys Gly Pro Ala Cys Thr Gly Cys Tyr | (SEQ ID NO: 547) |
| Cys Cys Glu Trp Cys Cys Ser Pro Ala Cys Thr Gly Cys Tyr | (SEQ ID NO: 548) |
| Cys Cys Glu Trp Cys Cys Thr Pro Ala Cys Thr Gly Cys Tyr | (SEQ ID NO: 549) |
| Cys Cys Glu Trp Cys Cys Cys Pro Ala Cys Thr Gly Cys Tyr | (SEQ ID NO: 550) |
| Cys Cys Glu Trp Cys Cys Gin Pro Ala Cys Thr Gly Cys Tyr | (SEQ ID NO: 551) |
| Cys Cys Glu Trp Cys Cys Tyr Pro Ala Cys Thr Gly Cys Tyr | (SEQ ID NO: 552) |
| Cys Cys Glu Trp Cys Cys Asp Pro Ala Cys Thr Gly Cys Tyr | (SEQ ID NO: 553) |
| Cys Cys Glu Trp Cys Cys Glu Pro Ala Cys Thr Gly Cys Tyr | (SEQ ID NO: 554) |
| Cys Cys Glu Trp Cys Cys Lys Pro Ala Cys Thr Gly Cys Tyr | (SEQ ID NO: 555) |
| Cys Cys Glu Trp Cys Cys Arg Pro Ala Cys Thr Gly Cys Tyr | (SEQ ID NO: 556) |
| Cys Cys Glu Trp Cys Cys His Pro Ala Cys Thr Gly Cys Tyr | (SEQ ID NO: 557) |
| Cys Cys Glu Trp Cys Cys Ala Pro Ala Cys Thr Gly Cys | (SEQ ID NO: 558) |
| Cys Cys Glu Trp Cys Cys Val Pro Ala Cys Thr Gly Cys | (SEQ ID NO: 559) |
| Cys Cys Glu Trp Cys Cys Leu Pro Ala Cys Thr Gly Cys | (SEQ ID NO: 560) |
| Cys Cys Glu Trp Cys Cys Ile Pro Ala Cys Thr Gly Cys | (SEQ ID NO: 561) |
| Cys Cys Glu Trp Cys Cys Pro Pro Ala Cys Thr Gly Cys | (SEQ ID NO: 562) |
| Cys Cys Glu Trp Cys Cys Met Pro Ala Cys Thr Gly Cys | (SEQ ID NO: 563) |
| Cys Cys Glu Trp Cys Cys Phe Pro Ala Cys Thr Gly Cys | (SEQ ID NO: 564) |
| Cys Cys Glu Trp Cys Cys Trp Pro Ala Cys Thr Gly Cys | (SEQ ID NO: 565) |
| Cys Cys Glu Trp Cys Cys Gly Pro Ala Cys Thr Gly Cys | (SEQ ID NO: 566) |
| Cys Cys Glu Trp Cys Cys Ser Pro Ala Cys Thr Gly Cys | (SEQ ID NO: 567) |
| Cys Cys Glu Trp Cys Cys Thr Pro Ala Cys Thr Gly Cys | (SEQ ID NO: 568) |
| Cys Cys Glu Trp Cys Cys Cys Pro Ala Cys Thr Gly Cys | (SEQ ID NO: 569) |
| Cys Cys Glu Trp Cys Cys Gln Pro Ala Cys Thr Gly Cys | (SEQ ID NO: 570) |
| Cys Cys Glu Trp Cys Cys Tyr Pro Ala Cys Thr Gly Cys | (SEQ ID NO: 571) |
| Cys Cys Glu Trp Cys Cys Asp Pro Ala Cys Thr Gly Cys | (SEQ ID NO: 572) |
| Cys Cys Glu Trp Cys Cys Glu Pro Ala Cys Thr Gly Cys | (SEQ ID NO: 573) |
| Cys Cys Glu Trp Cys Cys Lys Pro Ala Cys Thr Gly Cys | (SEQ ID NO: 574) |
| Cys Cys Glu Trp Cys Cys Arg Pro Ala Cys Thr Gly Cys | (SEQ ID NO: 575) |
| Cys Cys Glu Trp Cys Cys His Pro Ala Cys Thr Gly Cys | (SEQ ID NO: 576) |
| Cys Cys Glu Trp Cys Cys Asn Pro Ala Cys Thr Gly Cys Ala | (SEQ ID NO: 577) |
| Cys Cys Glu Trp Cys Cys Asn Pro Ala Cys Thr Gly Cys Val | (SEQ ID NO: 578) |
| Cys Cys Glu Trp Cys Cys Asn Pro Ala Cys Thr Gly Cys Leu | (SEQ ID NO: 579) |
| Cys Cys Glu Trp Cys Cys Asn Pro Ala Cys Thr Gly Cys Ile | (SEQ ID NO: 580) |
| Cys Cys Glu Trp Cys Cys Asn Pro Ala Cys Thr Gly Cys Pro | (SEQ ID NO: 581 |
| Cys Cys Glu Trp Cys Cys Asn Pro Ala Cys Thr Gly Cys Met | (SEQ ID NO: 58)2 |
| Cys Cys Glu Trp Cys Cys Asn Pro Ala Cys Thr Gly Cys Phe | (SEQ ID NO: 583) |
| Cys Cys Glu Trp Cys Cys Asn Pro Ala Cys Thr Gly Cys Trp | (SEQ ID NO: 584) |
| Cys Cys Glu Trp Cys Cys Asn Pro Ala Cys Thr Gly Cys Gly | (SEQ ID NO: 585) |
| Cys Cys Glu Trp Cys Cys Asn Pro Ala Cys Thr Gly Cys Ser | (SEQ ID NO: 586) |
| Cys Cys Glu Trp Cys Cys Asn Pro Ala Cys Thr Gly Cys Thr | (SEQ ID NO: 587) |
| Cys Cys Glu Trp Cys Cys Asn Pro Ala Cys Thr Gly Cys Cys | (SEQ ID NO: 588) |
| Cys Cys Glu Trp Cys Cys Asn Pro Ala Cys Thr Gly Cys Asn | (SEQ ID NO: 589) |
| Cys Cys Glu Trp Cys Cys Asn Pro Ala Cys Thr Gly Cys Gln | (SEQ ID NO: 590) |
| Cys Cys Glu Trp Cys Cys Asn Pro Ala Cys Thr Gly Cys Asp | (SEQ ID NO: 591) |
| Cys Cys Glu Trp Cys Cys Asn Pro Ala Cys Thr Gly Cys Glu | (SEQ ID NO: 592) |
| Cys Cys Glu Trp Cys Cys Asn Pro Ala Cys Thr Gly Cys Lys | (SEQ ID NO: 593) |
| Cys Cys Glu Trp Cys Cys Asn Pro Ala Cys Thr Gly Cys Arg | (SEQ ID NO: 594) |
| Cys Cys Glu Trp Cys Cys Asn Pro Ala Cys Thr Gly Cys His | (SEQ ID NO: 595) |
| Cys Cys Glu Phe Cys Cys Asn Pro Ala Cys Thr Gly Cys Ala | (SEQ ID NO: 596) |
| Cys Cys Glu Phe Cys Cys Asn Pro Ala Cys Thr Gly Cys Val | (SEQ ID NO: 597) |
| Cys Cys Glu Phe Cys Cys Asn Pro Ala Cys Thr Gly Cys Leu | (SEQ ID NO: 598) |
| Cys Cys Glu Phe Cys Cys Asn Pro Ala Cys Thr Gly Cys Ile | (SEQ ID NO: 599) |
| Cys Cys Glu Phe Cys Cys Asn Pro Ala Cys Thr Gly Cys Pro | (SEQ ID NO: 600) |
| Cys Cys Glu Phe Cys Cys Asn Pro Ala Cys Thr Gly Cys Met | (SEQ ID NO: 601) |
| Cys Cys Glu Phe Cys Cys Asn Pro Ala Cys Thr Gly Cys Phe | (SEQ ID NO: 602) |
| Cys Cys Glu Phe Cys Cys Asn Pro Ala Cys Thr Gly Cys Trp | (SEQ ID NO: 603) |
| Cys Cys Glu Phe Cys Cys Asn Pro Ala Cys Thr Gly Cys Gly | (SEQ ID NO: 604) |
| Cys Cys Glu Phe Cys Cys Asn Pro Ala Cys Thr Gly Cys Ser | (SEQ ID NO: 605) |
| Cys Cys Glu Phe Cys Cys Asn Pro Ala Cys Thr Gly Cys Thr | (SEQ ID NO: 606) |
| Cys Cys Glu Phe Cys Cys Asn Pro Ala Cys Thr Gly Cys Cys | (SEQ ID NO: 607) |
| Cys Cys Glu Phe Cys Cys Asn Pro Ala Cys Thr Gly Cys Asn | (SEQ ID NO: 608) |
| Cys Cys Glu Phe Cys Cys Asn Pro Ala Cys Thr Gly Cys Gln | (SEQ ID NO: 609) |
| Cys Cys Glu Phe Cys Cys Asn Pro Ala Cys Thr Gly Cys Asp | (SEQ ID NO: 610) |
| Cys Cys Glu Phe Cys Cys Asn Pro Ala Cys Thr Gly Cys Glu | (SEQ ID NO: 611) |
| Cys Cys Glu Phe Cys Cys Asn Pro Ala Cys Thr Gly Cys Lys | (SEQ ID NO: 612) |
| Cys Cys Glu Phe Cys Cys Asn Pro Ala Cys Thr Gly Cys Arg | (SEQ ID NO: 613) |
| Cys Cys Glu Phe Cys Cys Asn Pro Ala Cys Thr Gly Cys His | (SEQ ID NO: 614) |
| Cys Cys Ala Phe Cys Cys Asn Pro Ala Cys Thr Gly Cys Tyr | (SEQ ID NO: 615) |
| Cys Cys Val Phe Cys Cys Asn Pro Ala Cys Thr Gly Cys Tyr | (SEQ ID NO: 616) |
| Cys Cys Leu Phe Cys Cys Asn Pro Ala Cys Thr Gly Cys Tyr | (SEQ ID NO: 617) |
| Cys Cys Ile Phe Cys Cys Asn Pro Ala Cys Thr Gly Cys Tyr | (SEQ ID NO: 618) |
| Cys Cys Met Phe Cys Cys Asn Pro Ala Cys Thr Gly Cys Tyr | (SEQ ID NO: 619) |
| Cys Cys Phe Phe Cys Cys Asn Pro Ala Cys Thr Gly Cys Tyr | (SEQ ID NO: 620) |
| Cys Cys Trp Phe Cys Cys Asn Pro Ala Cys Thr Gly Cys Tyr | (SEQ ID NO: 621) |
| Cys Cys Gly Phe Cys Cys Asn Pro Ala Cys Thr Gly Cys Tyr | (SEQ ID NO: 622) |
| Cys Cys Ser Phe Cys Cys Asn Pro Ala Cys Thr Gly Cys Tyr | (SEQ ID NO: 623) |
| Cys Cys Thr Phe Cys Cys Asn Pro Ala Cys Thr Gly Cys Tyr | (SEQ ID NO: 624) |
| Cys Cys Cys Phe Cys Cys Asn Pro Ala Cys Thr Gly Cys Tyr | (SEQ ID NO: 625) |
| Cys Cys Asn Phe Cys Cys Asn Pro Ala Cys Thr Gly Cys Tyr | (SEQ ID NO: 626) |
| Cys Cys Gln Phe Cys Cys Asn Pro Ala Cys Thr Gly Cys Tyr | (SEQ ID NO: 627) |
| Cys Cys Tyr Phe Cys Cys Asn Pro Ala Cys Thr Gly Cys Tyr | (SEQ ID NO: 628) |
| Cys Cys Asp Phe Cys Cys Asn Pro Ala Cys Thr Gly Cys Tyr | (SEQ ID NO: 629) |
| Cys Cys Lys Phe Cys Cys Asn Pro Ala Cys Thr Gly Cys Tyr | (SEQ ID NO: 630) |
| Cys Cys Arg Phe Cys Cys Asn Pro Ala Cys Thr Gly Cys Tyr | (SEQ ID NO: 631) |
| Cys Cys His Phe Cys Cys Asn Pro Ala Cys Thr Gly Cys Tyr | (SEQ ID NO: 632) |
| Cys Cys Ala Phe Cys Cys Asn Pro Ala Cys Thr Gly Cys | (SEQ ID NO: 633) |
| Cys Cys Val Phe Cys Cys Asn Pro Ala Cys Thr Gly Cys | (SEQ ID NO: 634) |
| Cys Cys Leu Phe Cys Cys Asn Pro Ala Cys Thr Gly Cys | (SEQ ID NO: 635) |
| Cys Cys Ile Phe Cys Cys Asn Pro Ala Cys Thr Gly Cys | (SEQ ID NO: 636) |
| Cys Cys Met Phe Cys Cys Asn Pro Ala Cys Thr Gly Cys | (SEQ ID NO: 637) |
| Cys Cys Phe Phe Cys Cys Asn Pro Ala Cys Thr Gly Cys | (SEQ ID NO: 638) |
| Cys Cys Trp Phe Cys Cys Asn Pro Ala Cys Thr Gly Cys | (SEQ ID NO: 639) |
| Cys Cys Gly Phe Cys Cys Asn Pro Ala Cys Thr Gly Cys | (SEQ ID NO: 640) |
| Cys Cys Ser Phe Cys Cys Asn Pro Ala Cys Thr Gly Cys | (SEQ ID NO: 641) |
| Cys Cys Thr Phe Cys Cys Asn Pro Ala Cys Thr Gly Cys | (SEQ ID NO: 642) |
| Cys Cys Cys Phe Cys Cys Asn Pro Ala Cys Thr Gly Cys | (SEQ ID NO: 643) |
| Cys Cys Asn Phe Cys Cys Asn Pro Ala Cys Thr Gly Cys | (SEQ ID NO: 644) |
| Cys Cys Gln Phe Cys Cys Asn Pro Ala Cys Thr Gly Cys | (SEQ ID NO: 645) |
| Cys Cys Tyr Phe Cys Cys Asn Pro Ala Cys Thr Gly Cys | (SEQ ID NO: 646) |
| Cys Cys Asp Phe Cys Cys Asn Pro Ala Cys Thr Gly Cys | (SEQ ID NO: 647) |
| Cys Cys Lys Phe Cys Cys Asn Pro Ala Cys Thr Gly Cys | (SEQ ID NO: 648) |
| Cys Cys Arg Phe Cys Cys Asn Pro Ala Cys Thr Gly Cys | (SEQ ID NO: 649) |
| Cys Cys His Phe Cys Cys Asn Pro Ala Cys Thr Gly Cys | (SEQ ID NO: 650) |
| Cys Cys Ala Trp Cys Cys Asn Pro Ala Cys Thr Gly Cys Tyr | (SEQ ID NO: 651) |
| Cys Cys Val Trp Cys Cys Asn Pro Ala Cys Thr Gly Cys Tyr | (SEQ ID NO: 652) |
| Cys Cys Leu Trp Cys Cys Asn Pro Ala Cys Thr Gly Cys Tyr | (SEQ ID NO: 653) |
| Cys Cys Ile Trp Cys Cys Asn Pro Ala Cys Thr Gly Cys Tyr | (SEQ ID NO: 654) |
| Cys Cys Met Trp Cys Cys Asn Pro Ala Cys Thr Gly Cys Tyr | (SEQ ID NO: 655) |
| Cys Cys Phe Trp Cys Cys Asn Pro Ala Cys Thr Gly Cys Tyr | (SEQ ID NO: 656) |
| Cys Cys Trp Trp Cys Cys Asn Pro Ala Cys Thr Gly Cys Tyr | (SEQ ID NO: 657) |
| Cys Cys Gly Trp Cys Cys Asn Pro Ala Cys Thr Gly Cys Tyr | (SEQ ID NO: 658) |
| Cys Cys Ser Trp Cys Cys Asn Pro Ala Cys Thr Gly Cys Tyr | (SEQ ID NO: 659) |
| Cys Cys Thr Trp Cys Cys Asn Pro Ala Cys Thr Gly Cys Tyr | (SEQ ID NO: 660) |
| Cys Cys Cys Trp Cys Cys Asn Pro Ala Cys Thr Gly Cys Tyr | (SEQ ID NO: 661) |
| Cys Cys Asn Trp Cys Cys Asn Pro Ala Cys Thr Gly Cys Tyr | (SEQ ID NO: 662) |
| Cys Cys Gln Trp Cys Cys Asn Pro Ala Cys Thr Gly Cys Tyr | (SEQ ID NO: 663) |
| Cys Cys Tyr Trp Cys Cys Asn Pro Ala Cys Thr Gly Cys Tyr | (SEQ ID NO: 664) |
| Cys Cys Asp Trp Cys Cys Asn Pro Ala Cys Thr Gly Cys Tyr | (SEQ ID NO: 665) |
| Cys Cys Lys Trp Cys Cys Asn Pro Ala Cys Thr Gly Cys Tyr | (SEQ ID NO: 666) |
| Cys Cys Arg Trp Cys Cys Asn Pro Ala Cys Thr Gly Cys Tyr | (SEQ ID NO: 667) |
| Cys Cys His Trp Cys Cys Asn Pro Ala Cys Thr Gly Cys Tyr | (SEQ ID NO: 668) |
| Cys Cys Ala Trp Cys Cys Asn Pro Ala Cys Thr Gly Cys | (SEQ ID NO: 669) |
| Cys Cys Val Trp Cys Cys Asn Pro Ala Cys Thr Gly Cys | (SEQ ID NO: 670) |
| Cys Cys Leu Trp Cys Cys Asn Pro Ala Cys Thr Gly Cys | (SEQ ID NO: 671) |
| Cys Cys Ile Trp Cys Cys Asn Pro Ala Cys Thr Gly Cys | (SEQ ID NO: 672) |
| Cys Cys Met Trp Cys Cys Asn Pro Ala Cys Thr Gly Cys | (SEQ ID NO: 673) |
| Cys Cys Phe Trp Cys Cys Asn Pro Ala Cys Thr Gly Cys | (SEQ ID NO: 674) |
| Cys Cys Trp Trp Cys Cys Asn Pro Ala Cys Thr Gly Cys | (SEQ ID NO: 675) |
| Cys Cys Gly Trp Cys Cys Asn Pro Ala Cys Thr Gly Cys | (SEQ ID NO: 676) |
| Cys Cys Ser Trp Cys Cys Asn Pro Ala Cys Thr Gly Cys | (SEQ ID NO: 677) |
| Cys Cys Thr Trp Cys Cys Asn Pro Ala Cys Thr Gly Cys | (SEQ ID NO: 678) |
| Cys Cys Cys Trp Cys Cys Asn Pro Ala Cys Thr Gly Cys | (SEQ ID NO: 679) |
| Cys Cys Asn Trp Cys Cys Asn Pro Ala Cys Thr Gly Cys | (SEQ ID NO: 680) |
| Cys Cys Gln Trp Cys Cys Asn Pro Ala Cys Thr Gly Cys | (SEQ ID NO: 681) |
| Cys Cys Tyr Trp Cys Cys Asn Pro Ala Cys Thr Gly Cys | (SEQ ID NO: 682) |
| Cys Cys Asp Trp Cys Cys Asn Pro Ala Cys Thr Gly Cys | (SEQ ID NO: 683) |
| Cys Cys Lys Trp Cys Cys Asn Pro Ala Cys Thr Gly Cys | (SEQ ID NO: 684) |
| Cys Cys Arg Trp Cys Cys Asn Pro Ala Cys Thr Gly Cys | (SEQ ID NO: 685) |
| Cys Cys His Trp Cys Cys Asn Pro Ala Cys Thr Gly Cys | (SEQ ID NO: 686) |
| Cys Glu Leu Cys Ile Asn Val Ala Cys Thr Gly Cys | (SEQ ID NO: 687) |
| Cys Glu Leu Cys Val Asn Val Ala Cys Thr Gly Cys | (SEQ ID NO: 688) |
| Cys Ala Glu Leu Cys Cys Asn Pro Ala Cys | (SEQ ID NO: 689) |
| Cys Cys Gly Leu Cys Cys Asn Pro Ala Cys Ala Gly Cys | (SEQ ID NO: 690) |
| Cys Cys Gly Leu Cys Cys Tyr Pro Ala Cys Ala Gly Cys | (SEQ ID NO: 691) |
| Cys Glu Leu Cys Cys Asn Pro Ala Cys Ala Gly Cys | (SEQ ID NO: 692) |
| Cys Cys Asp Val Cys Cys Tyr Pro Ala Cys Thr Gly Cys | (SEQ ID NO: 693) |
| Cys Cys Glu Leu Cys Cys Asn Pro Ala Cys Ala Gly Cys | (SEQ ID NO: 694) |
| Cys Cys Glu Leu Cys Cys Tyr Pro Ala Cys Ala Gly Cys | (SEQ ID NO: 695) |
| Cys Cys Glu Leu Cys Cys Asn Pro Ala Cys Thr Gly Cys | (SEQ ID NO: 696) |
| Cys Cys Glu Leu Cys Cys Tyr Pro Ala Cys Thr Gly Cys | (SEQ ID NO: 697) |
| Cys Cys Glu Leu Cys Cys Asn Pro Gly Cys Thr Gly Cys | (SEQ ID NO: 698) |
| Cys Cys Glu Ala Cys Cys Asn Pro Ala Cys Thr Gly Cys | (SEQ ID NO: 699) |
| Cys Cys Glu Lys Cys Cys Asn Pro Ala Cys Thr Gly Cys | (SEQ ID NO: 700) |
| Cys Cys Glu Leu Cys Cys Asn Pro Ala Cys Thr Ala Cys | (SEQ ID NO: 701) |
| Cys Cys Pro Leu Cys Cys Asn Pro Ala Cys Thr Gly Cys | (SEQ ID NO: 702) |
| Ala Cys Glu Leu Cys Ala Asn Pro Ala Cys Thr Gly Cys | (SEQ ID NO: 703) |
| Cys Cys Glu Leu Ala Cys Asn Pro Ala Cys Thr Gly Ala | (SEQ ID NO: 704) |
| Cys Glu Leu Cys Ala Asn Pro Ala Cys Thr Gly Cys | (SEQ ID NO: 705) |
| Cys Cys Glu Leu Ala Cys Asn Pro Ala Cys | (SEQ ID NO: 706) |
| Cys Cys Asp Val Cys Cys Asn Pro Ala Cys Ala Gly Cys | (SEQ ID NO: 707) |
| Cys Cys Asp Val Cys Cys Asn Pro Ala Cys Thr Gly Cys | (SEQ ID NO: 708) |
| Cys Cys Asp Val Cys Cys Asn Pro Ala Cys Ala Gly Cys Tyr | (SEQ ID NO: 709) |
| Cys Cys Asp Val Cys Cys Asn Pro Ala Cys Thr Gly Cys Tyr | (SEQ ID NO: 710) |
| Cys Cys Glu Leu Cys Cys Tyr Pro Ala Cys Ala Gly Cys | (SEQ ID NO: 711) |
| Cys Cys Ile Cys Cys Asn Pro Ala Cys Phe Gly Cys | (SEQ ID NO: 712) |
| Cys Cys Asn Tyr Cys Cys Ser Pro Cys Gly Cys | (SEQ ID NO: 713) |
| Cys Cys Asp Val Cys Cys Asn Pro Ala Cys Ala Gly Cys | (SEQ ID NO. 714) |
| Cys Cys Asp Ala Cys Cys Asn Pro Ala Cys Ala Gly Cys | (SEQ ID NO. 715) |
| Cys Cys Asp Cys Cys Cys Asn Pro Ala Cys Ala Gly Cys | (SEQ ID NO. 716) |
| Cys Cys Asp Asp Cys Cys Asn Pro Ala Cys Ala Gly Cys | (SEQ ID NO. 717) |
| Cys Cys Asp Glu Cys Cys Asn Pro Ala Cys Ala Gly Cys | (SEQ ID NO. 718) |
| Cys Cys Asp Phe Cys Cys Asn Pro Ala Cys Ala Gly Cys | (SEQ ID NO. 719) |
| Cys Cys Asp Gly Cys Cys Asn Pro Ala Cys Ala Gly Cys | (SEQ ID NO. 720) |
| Cys Cys Asp His Cys Cys Asn Pro Ala Cys Ala Gly Cys | (SEQ ID NO. 721) |
| Cys Cys Asp Ile Cys Cys Asn Pro Ala Cys Ala Gly Cys | (SEQ ID NO. 722) |
| Cys Cys Asp Lys Cys Cys Asn Pro Ala Cys Ala Gly Cys | (SEQ ID NO. 723) |
| Cys Cys Asp Leu Cys Cys Asn Pro Ala Cys Ala Gly Cys | (SEQ ID NO. 724) |
| Cys Cys Asp Met Cys Cys Asn Pro Ala Cys Ala Gly Cys | (SEQ ID NO. 725) |
| Cys Cys Asp Asn Cys Cys Asn Pro Ala Cys Ala Gly Cys | (SEQ ID NO. 726) |
| Cys Cys Asp Pro Cys Cys Asn Pro Ala Cys Ala Gly Cys | (SEQ ID NO. 727) |
| Cys Cys Asp Gln Cys Cys Asn Pro Ala Cys Ala Gly Cys | (SEQ ID NO. 728) |
| Cys Cys Asp Arg Cys Cys Asn Pro Ala Cys Ala Gly Cys | (SEQ ID NO. 729) |
| Cys Cys Asp Ser Cys Cys Asn Pro Ala Cys Ala Gly Cys | (SEQ ID NO. 730) |
| Cys Cys Asp Thr Cys Cys Asn Pro Ala Cys Ala Gly Cys | (SEQ ID NO. 731) |
| Cys Cys Asp Trp Cys Cys Asn Pro Ala Cys Ala Gly Cys | (SEQ ID NO. 732) |
| Cys Cys Asp Tyr Cys Cys Asn Pro Ala Cys Ala Gly Cys | (SEQ ID NO. 733) |
| Cys Cys Glu Ile Cys Cys Asn Pro Ala Cys Phe Gly Cys | (SEQ ID NO. 734) |
| Cys Cys Glu Ala Cys Cys Asn Pro Ala Cys Phe Gly Cys | (SEQ ID NO. 735) |
| Cys Cys Glu Cys Cys Cys Asn Pro Ala Cys Phe Gly Cys | (SEQ ID NO. 736) |
| Cys Cys Glu Asp Cys Cys Asn Pro Ala Cys Phe Gly Cys | (SEQ ID NO. 737) |
| Cys Cys Glu Glu Cys Cys Asn Pro Ala Cys Phe Gly Cys | (SEQ ID NO. 738) |
| Cys Cys Glu Phe Cys Cys Asn Pro Ala Cys Phe Gly Cys | (SEQ ID NO. 739) |
| Cys Cys Glu Gly Cys Cys Asn Pro Ala Cys Phe Gly Cys | (SEQ ID NO. 740) |
| Cys Cys Glu His Cys Cys Asn Pro Ala Cys Phe Gly Cys | (SEQ ID NO. 741) |
| Cys Cys Glu Lys Cys Cys Asn Pro Ala Cys Phe Gly Cys | (SEQ ID NO. 742) |
| Cys Cys Glu Leu Cys Cys Asn Pro Ala Cys Phe Gly Cys | (SEQ ID NO. 743) |
| Cys Cys Glu Met Cys Cys Asn Pro Ala Cys Phe Gly Cys | (SEQ ID NO. 744) |
| Cys Cys Glu Asn Cys Cys Asn Pro Ala Cys Phe Gly Cys | (SEQ ID NO. 745) |
| Cys Cys Glu Pro Cys Cys Asn Pro Ala Cys Phe Gly Cys | (SEQ ID NO. 746) |
| Cys Cys Glu Gln Cys Cys Asn Pro Ala Cys Phe Gly Cys | (SEQ ID NO. 747) |
| Cys Cys Glu Arg Cys Cys Asn Pro Ala Cys Phe Gly Cys | (SEQ ID NO. 748) |
| Cys Cys Glu Ser Cys Cys Asn Pro Ala Cys Phe Gly Cys | (SEQ ID NO. 749) |
| Cys Cys Glu Thr Cys Cys Asn Pro Ala Cys Phe Gly Cys | (SEQ ID NO. 750) |
| Cys Cys Glu Val Cys Cys Asn Pro Ala Cys Phe Gly Cys | (SEQ ID NO. 751) |
| Cys Cys Glu Trp Cys Cys Asn Pro Ala Cys Phe Gly Cys | (SEQ ID NO. 752) |
| Cys Cys Glu Tyr Cys Cys Asn Pro Ala Cys Phe Gly Cys | (SEQ ID NO. 753) |
| Cys Cys Glu Leu Cys Cys Asn Pro Ala Cys Ala Pro Cys | (SEQ ID NO. 754) |
| Cys Cys Glu Ala Cys Cys Asn Pro Ala Cys Ala Pro Cys | (SEQ ID NO. 755) |
| Cys Cys Glu Cys Cys Cys Asn Pro Ala Cys Ala Pro Cys | (SEQ ID NO. 756) |
| Cys Cys Glu Asp Cys Cys Asn Pro Ala Cys Ala Pro Cys | (SEQ ID NO. 757) |
| Cys Cys Glu Glu Cys Cys Asn Pro Ala Cys Ala Pro Cys | (SEQ ID NO. 758) |
| Cys Cys Glu Phe Cys Cys Asn Pro Ala Cys Ala Pro Cys | (SEQ ID NO. 759) |
| Cys Cys Glu Gly Cys Cys Asn Pro Ala Cys Ala Pro Cys | (SEQ ID NO. 760) |
| Cys Cys Glu His Cys Cys Asn Pro Ala Cys Ala Pro Cys | (SEQ ID NO. 761) |
| Cys Cys Glu Ile Cys Cys Asn Pro Ala Cys Ala Pro Cys | (SEQ ID NO. 762) |
| Cys Cys Glu Lys Cys Cys Asn Pro Ala Cys Ala Pro Cys | (SEQ ID NO. 763) |
| Cys Cys Glu Met Cys Cys Asn Pro Ala Cys Ala Pro Cys | (SEQ ID NO. 764) |
| Cys Cys Glu Asn Cys Cys Asn Pro Ala Cys Ala Pro Cys | (SEQ ID NO. 765) |
| Cys Cys Glu Pro Cys Cys Asn Pro Ala Cys Ala Pro Cys | (SEQ ID NO. 766) |
| Cys Cys Glu Gln Cys Cys Asn Pro Ala Cys Ala Pro Cys | (SEQ ID NO. 767) |
| Cys Cys Glu Arg Cys Cys Asn Pro Ala Cys Ala Pro Cys | (SEQ ID NO. 768) |
| Cys Cys Glu Ser Cys Cys Asn Pro Ala Cys Ala Pro Cys | (SEQ ID NO. 769) |
| Cys Cys Glu Thr Cys Cys Asn Pro Ala Cys Ala Pro Cys | (SEQ ID NO. 770) |
| Cys Cys Glu Val Cys Cys Asn Pro Ala Cys Ala Pro Cys | (SEQ ID NO. 771) |
| Cys Cys Glu Trp Cys Cys Asn Pro Ala Cys Ala Pro Cys | (SEQ ID NO. 772) |
| Cys Cys Glu Tyr Cys Cys Asn Pro Ala Cys Ala Pro Cys | (SEQ ID NO. 773) |
| Cys Cys Glu Leu Cys Cys Asn Pro Ala Cys Phe Gly Cys | (SEQ ID NO. 774) |
| Cys Cys Glu Ala Cys Cys Asn Pro Ala Cys Phe Gly Cys | (SEQ ID NO. 775) |
| Cys Cys Glu Cys Cys Cys Asn Pro Ala Cys Phe Gly Cys | (SEQ ID NO. 776) |
| Cys Cys Glu Asp Cys Cys Asn Pro Ala Cys Phe Gly Cys | (SEQ ID NO. 777) |
| Cys Cys Glu Glu Cys Cys Asn Pro Ala Cys Phe Gly Cys | (SEQ ID NO. 778) |
| Cys Cys Glu Phe Cys Cys Asn Pro Ala Cys Phe Gly Cys | (SEQ ID NO. 779) |
| Cys Cys Glu Gly Cys Cys Asn Pro Ala Cys Phe Gly Cys | (SEQ ID NO. 780) |
| Cys Cys Glu His Cys Cys Asn Pro Ala Cys Phe Gly Cys | (SEQ ID NO. 781) |
| Cys Cys Glu Ile Cys Cys Asn Pro Ala Cys Phe Gly Cys | (SEQ ID NO. 782) |
| Cys Cys Glu Lys Cys Cys Asn Pro Ala Cys Phe Gly Cys | (SEQ ID NO. 783) |
| Cys Cys Glu Met Cys Cys Asn Pro Ala Cys Phe Gly Cys | (SEQ ID NO. 784) |
| Cys Cys Glu Asn Cys Cys Asn Pro Ala Cys Phe Gly Cys | (SEQ ID NO. 785) |
| Cys Cys Glu Pro Cys Cys Asn Pro Ala Cys Phe Gly Cys | (SEQ ID NO. 786) |
| Cys Cys Glu Gln Cys Cys Asn Pro Ala Cys Phe Gly Cys | (SEQ ID NO. 787) |
| Cys Cys Glu Arg Cys Cys Asn Pro Ala Cys Phe Gly Cys | (SEQ ID NO. 788) |
| Cys Cys Glu Ser Cys Cys Asn Pro Ala Cys Phe Gly Cys | (SEQ ID NO. 789) |
| Cys Cys Glu Thr Cys Cys Asn Pro Ala Cys Phe Gly Cys | (SEQ ID NO. 790) |
| Cys Cys Glu Val Cys Cys Asn Pro Ala Cys Phe Gly Cys | (SEQ ID NO. 791) |
| Cys Cys Glu Trp Cys Cys Asn Pro Ala Cys Phe Gly Cys | (SEQ ID NO. 792) |
| Cys Cys Glu Tyr Cys Cys Asn Pro Ala Cys Phe Gly Cys | (SEQ ID NO. 793) |
| Cys Cys Glu Val Cys Cys Asn Pro Ala Cys Ala Gly Cys | (SEQ ID NO. 794) |
| Cys Cys Glu Ala Cys Cys Asn Pro Ala Cys Ala Gly Cys | (SEQ ID NO. 795) |
| Cys Cys Glu Cys Cys Cys Asn Pro Ala Cys Ala Gly Cys | (SEQ ID NO. 796) |
| Cys Cys Glu Asp Cys Cys Asn Pro Ala Cys Ala Gly Cys | (SEQ ID NO. 797) |
| Cys Cys Glu Glu Cys Cys Asn Pro Ala Cys Ala Gly Cys | (SEQ ID NO. 798) |
| Cys Cys Glu Phe Cys Cys Asn Pro Ala Cys Ala Gly Cys | (SEQ ID NO. 799) |
| Cys Cys Glu Gly Cys Cys Asn Pro Ala Cys Ala Gly Cys | (SEQ ID NO. 800) |
| Cys Cys Glu His Cys Cys Asn Pro Ala Cys Ala Gly Cys | (SEQ ID NO. 801) |
| Cys Cys Glu Ile Cys Cys Asn Pro Ala Cys Ala Gly Cys | (SEQ ID NO. 802) |
| Cys Cys Glu Lys Cys Cys Asn Pro Ala Cys Ala Gly Cys | (SEQ ID NO. 803) |
| Cys Cys Glu Leu Cys Cys Asn Pro Ala Cys Ala Gly Cys | (SEQ ID NO. 804) |
| Cys Cys Glu Met Cys Cys Asn Pro Ala Cys Ala Gly Cys | (SEQ ID NO. 210) |
| Cys Cys Glu Asn Cys Cys Asn Pro Ala Cys Ala Gly Cys | (SEQ ID NO. 211) |
| Cys Cys Glu Pro Cys Cys Asn Pro Ala Cys Ala Gly Cys | (SEQ ID NO. 212) |
| Cys Cys Glu Gln Cys Cys Asn Pro Ala Cys Ala Gly Cys | (SEQ ID NO. 213) |
| Cys Cys Glu Arg Cys Cys Asn Pro Ala Cys Ala Gly Cys | (SEQ ID NO. 214) |
| Cys Cys Glu Ser Cys Cys Asn Pro Ala Cys Ala Gly Cys | (SEQ ID NO. 215) |
| Cys Cys Glu Thr Cys Cys Asn Pro Ala Cys Ala Gly Cys | (SEQ ID NO. 216) |
| Cys Cys Glu Trp Cys Cys Asn Pro Ala Cys Ala Gly Cys | (SEQ ID NO. 217) |
| Cys Cys Glu Tyr Cys Cys Asn Pro Ala Cys Ala Gly Cys | (SEQ ID NO. 218) |
| Cys Cys Glu Leu Cys Cys Asn Pro Ala Cys Thr Gly Cys Tyr | (SEQ ID NO: 219) |
| Cys Cys Glu Tyr Cys Cys Asn Pro Ala Cys Thr Gly Cys Tyr | (SEQ ID NO: 805) |
| and | |
| Cys Cys Glu Leu Cys Cys Asn Pro Ala Cys Thr Gly Cys. | (SEQ ID NO: 806) |

In some cases: D′ comprises an amino acid sequence selected from SEQ ID NOs: 807-847, respectively, as shown below:

(SEQ ID NO: 807)
Cys Cys Glu Ala Cys Cys Asn Pro Ala Cys Thr
Gly Cys
(SEQ ID NO: 808)
Cys Cys Glu Arg Cys Cys Asn Pro Ala Cys Thr
Gly Cys
(SEQ ID NO: 809)
Cys Cys Glu Asn Cys Cys Asn Pro Ala Cys Thr
Gly Cys
(SEQ ID NO: 810)
Cys Cys Glu Asp Cys Cys Asn Pro Ala Cys Thr
Gly Cys
(SEQ ID NO: 811)
Cys Cys Glu Cys Cys Cys Asn Pro Ala Cys Thr
Gly Cys
(SEQ ID NO: 812)
Cys Cys Glu Gln Cys Cys Asn Pro Ala Cys Thr
Gly Cys
(SEQ ID NO: 813)
Cys Cys Glu Glu Cys Cys Asn Pro Ala Cys Thr
Gly Cys
(SEQ ID NO: 814)
Cys Cys Glu Gly Cys Cys Asn Pro Ala Cys Thr
Gly Cys
(SEQ ID NO: 815)
Cys Cys Glu His Cys Cys Asn Pro Ala Cys Thr
Gly Cys
(SEQ ID NO: 816)
Cys Cys Glu Ile Cys Cys Asn Pro Ala Cys Thr
Gly Cys
(SEQ ID NO: 817)
Cys Cys Glu Lys Cys Cys Asn Pro Ala Cys Thr
Gly Cys
(SEQ ID NO: 818)
Cys Cys Glu Met Cys Cys Asn Pro Ala Cys Thr
Gly Cys
(SEQ ID NO: 819)
Cys Cys Glu Phe Cys Cys Asn Pro Ala Cys Thr
Gly Cys
(SEQ ID NO: 820)
Cys Cys Glu Pro Cys Cys Asn Pro Ala Cys Thr
Gly Cys
(SEQ ID NO: 821)
Cys Cys Glu Ser Cys Cys Asn Pro Ala Cys Thr
Gly Cys
(SEQ ID NO: 822)
Cys Cys Glu Thr Cys Cys Asn Pro Ala Cys Thr
Gly Cys
(SEQ ID NO: 823)
Cys Cys Glu Trp Cys Cys Asn Pro Ala Cys Thr
Gly Cys
(SEQ ID NO: 824)
Cys Cys Glu Val Cys Cys Asn Pro Ala Cys Thr
Gly Cys
(SEQ ID NO: 825)
Cys Cys Glu Tyr Cys Cys Asn Pro Ala Cys Thr
Gly Cys
(SEQ ID NO: 826)
Cys Cys Glu Leu Cys Cys Asn Pro Ala Cys Thr
Gly Cys
(SEQ ID NO: 827)
Cys Cys Glu Ala Cys Cys Asn Pro Ala Cys Thr
Gly Cys Tyr
(SEQ ID NO: 828)
Cys Cys Glu Arg Cys Cys Asn Pro Ala Cys Thr
Gly Cys Tyr
(SEQ ID NO: 829)
Cys Cys Glu Asn Cys Cys Asn Pro Ala Cys Thr
Gly Cys Tyr
(SEQ ID NO: 830)
Cys Cys Glu Asp Cys Cys Asn Pro Ala Cys Thr
Gly Cys Tyr
(SEQ ID NO: 831)
Cys Cys Glu Cys Cys Cys Asn Pro Ala Cys Thr
Gly Cys Tyr
(SEQ ID NO: 832)
Cys Cys Glu Gln Cys Cys Asn Pro Ala Cys Thr
Gly Cys Tyr
(SEQ ID NO: 833)
Cys Cys Glu Glu Cys Cys Asn Pro Ala Cys Thr
Gly Cys Tyr
(SEQ ID NO: 834)
Cys Cys Glu Gly Cys Cys Asn Pro Ala Cys Thr
Gly Cys Tyr
(SEQ ID NO: 835)
Cys Cys Glu His Cys Cys Asn Pro Ala Cys Thr
Gly Cys Tyr
(SEQ ID NO: 836)
Cys Cys Glu Ile Cys Cys Asn Pro Ala Cys Thr
Gly Cys Tyr
(SEQ ID NO: 837)
Cys Cys Glu Lys Cys Cys Asn Pro Ala Cys Thr
Gly Cys Tyr
(SEQ ID NO: 838)
Cys Cys Glu Met Cys Cys Asn Pro Ala Cys Thr
Gly Cys Tyr
(SEQ ID NO: 839)
Cys Cys Glu Phe Cys Cys Asn Pro Ala Cys Thr
Gly Cys Tyr
(SEQ ID NO: 840)
Cys Cys Glu Pro Cys Cys Asn Pro Ala Cys Thr
Gly Cys Tyr
(SEQ ID NO: 841)
Cys Cys Glu Ser Cys Cys Asn Pro Ala Cys Thr
Gly Cys Tyr
(SEQ ID NO: 842)
Cys Cys Glu Thr Cys Cys Asn Pro Ala Cys Thr
Gly Cys Tyr
(SEQ ID NO: 843)
Cys Cys Glu Trp Cys Cys Asn Pro Ala Cys Thr
Gly Cys Tyr
(SEQ ID NO: 844)
Cys Cys Glu Val Cys Cys Asn Pro Ala Cys Thr
Gly Cys Tyr
(SEQ ID NO: 845)
Cys Cys Glu Tyr Cys Cys Asn Pro Ala Cys Thr
Gly Cys Tyr
(SEQ ID NO: 846)
Cys Cys Glu Leu Cys Cys Asn Pro Ala Cys Thr
Gly Cys Tyr

Among the useful peptides are peptides comprising, consisting of or consisting essentially of the amino acid sequence Xaa₁ Xaa₂ Xaa₃ Xaa₄ Xaa₅ Cys Cys Glu Xaa₉ Cys Cys Asn Pro Ala Cys Thr Gly Cys Tyr Xaa₂₀ Xaa₂₁ (SEQ ID NO:64) are the following peptides (SEQ ID NOs: 848-910, respectively, as shown below):

(SEQ ID NO: 849)
Gln Ser Ser Asn Tyr Cys Cys Glu Tyr Cys Cys
Asn Pro Ala Cys Thr Gly Cys Tyr
(SEQ ID NO: 850)
Asn Thr Ser Asn Tyr Cys Cys Glu Tyr Cys Cys
Asn Pro Ala Cys Thr Gly Cys Tyr
(SEQ ID NO: 851)
Asn Leu Ser Asn Tyr Cys Cys Glu Tyr Cys Cys
Asn Pro Ala Cys Thr Gly Cys Tyr
(SEQ ID NO: 852)
Asn Ile Ser Asn Tyr Cys Cys Glu Tyr Cys Cys
Asn Pro Ala Cys Thr Gly Cys Tyr
(SEQ ID NO: 853)
Asn Ser Ser Gln Tyr Cys Cys Glu Tyr Cys Cys
Asn Pro Ala Cys Thr Gly Cys Tyr
(SEQ ID NO: 854)
Ser Ser Asn Tyr Cys Cys Glu Tyr Cys Cys Asn
Pro Ala Cys Thr Gly Cys Tyr
(SEQ ID NO: 855)
Gln Ser Ser Gln Tyr Cys Cys Glu Tyr Cys Cys
Asn Pro Ala Cys Thr Gly Cys Tyr
(SEQ ID NO: 856)
Ser Ser Gln Tyr Cys Cys Glu Tyr Cys Cys Asn
Pro Ala Cys Thr Gly Cys Tyr
(SEQ ID NO: 857)
Asn Ser Ser Asn Tyr Cys Cys Glu Ala Cys Cys
Asn Pro Ala Cys Thr Gly Cys Tyr
(SEQ ID NO: 858)
Asn Ser Ser Asn Tyr Cys Cys Glu Arg Cys Cys
Asn Pro Ala Cys Thr Gly Cys Tyr
(SEQ ID NO: 859)
Asn Ser Ser Asn Tyr Cys Cys Glu Asn Cys Cys
Asn Pro Ala Cys Thr Gly Cys Tyr
(SEQ ID NO: 860)
Asn Ser Ser Asn Tyr Cys Cys Glu Asp Cys Cys
Asn Pro Ala Cys Thr Gly Cys Tyr
(SEQ ID NO: 861)
Asn Ser Ser Asn Tyr Cys Cys Glu Cys Cys Cys
Asn Pro Ala Cys Thr Gly Cys Tyr
(SEQ ID NO: 862)
Asn Ser Ser Asn Tyr Cys Cys Glu Gln Cys Cys
Asn Pro Ala Cys Thr Gly Cys Tyr
(SEQ ID NO: 863)
Asn Ser Ser Asn Tyr Cys Cys Glu Glu Cys Cys
Asn Pro Ala Cys Thr Gly Cys Tyr
(SEQ ID NO: 864)
Asn Ser Ser Asn Tyr Cys Cys Glu Gly Cys Cys
Asn Pro Ala Cys Thr Gly Cys Tyr
(SEQ ID NO: 865)
Asn Ser Ser Asn Tyr Cys Cys Glu His Cys Cys
Asn Pro Ala Cys Thr Gly Cys Tyr
(SEQ ID NO: 866)
Asn Ser Ser Asn Tyr Cys Cys Glu Ile Cys Cys
Asn Pro Ala Cys Thr Gly Cys Tyr
(SEQ ID NO: 867)
Asn Ser Ser Asn Tyr Cys Cys Glu Lys Cys Cys
Asn Pro Ala Cys Thr Gly Cys Tyr
(SEQ ID NO: 868)
Asn Ser Ser Asn Tyr Cys Cys Glu Met Cys Cys
Asn Pro Ala Cys Thr Gly Cys Tyr
(SEQ ID NO: 869)
Asn Ser Ser Asn Tyr Cys Cys Glu Phe Cys Cys
Asn Pro Ala Cys Thr Gly Cys Tyr
(SEQ ID NO: 870)
Asn Ser Ser Asn Tyr Cys Cys Glu Pro Cys Cys
Asn Pro Ala Cys Thr Gly Cys Tyr
(SEQ ID NO: 871)
Asn Ser Ser Asn Tyr Cys Cys Glu Ser Cys Cys
Asn Pro Ala Cys Thr Gly Cys Tyr
(SEQ ID NO: 872)
Asn Ser Ser Asn Tyr Cys Cys Glu Thr Cys Cys
Asn Pro Ala Cys Thr Gly Cys Tyr
(SEQ ID NO: 873)
Asn Ser Ser Asn Tyr Cys Cys Glu Trp Cys Cys
Asn Pro Ala Cys Thr Gly Cys Tyr
(SEQ ID NO: 874)
Asn Ser Ser Asn Tyr Cys Cys Glu Val Cys Cys
Asn Pro Ala Cys Thr Gly Cys Tyr
(SEQ ID NO: 875)
Cys Cys Glu Ala Cys Cys Asn Pro Ala Cys Thr
Gly Cys Tyr
(SEQ ID NO: 876)
Cys Cys Glu Arg Cys Cys Asn Pro Ala Cys Thr
Gly Cys Tyr
(SEQ ID NO: 877)
Cys Cys Glu Asn Cys Cys Asn Pro Ala Cys Thr
Gly Cys Tyr
(SEQ ID NO: 878)
Cys Cys Glu Asp Cys Cys Asn Pro Ala Cys Thr
Gly Cys Tyr
(SEQ ID NO: 879)
Cys Cys Glu Cys Cys Cys Asn Pro Ala Cys Thr
Gly Cys Tyr
(SEQ ID NO: 880)
Cys Cys Glu Gln Cys Cys Asn Pro Ala Cys Thr
Gly Cys Tyr
(SEQ ID NO: 881)
Cys Cys Glu Glu Cys Cys Asn Pro Ala Cys Thr
Gly Cys Tyr
(SEQ ID NO: 882)
Cys Cys Glu Gly Cys Cys Asn Pro Ala Cys Thr
Gly Cys Tyr
(SEQ ID NO: 883)
Cys Cys Glu His Cys Cys Asn Pro Ala Cys Thr
Gly Cys Tyr
(SEQ ID NO: 884)
Cys Cys Glu Ile Cys Cys Asn Pro Ala Cys Thr
Gly Cys Tyr
(SEQ ID NO: 885)
Cys Cys Glu Lys Cys Cys Asn Pro Ala Cys Thr
Gly Cys Tyr
(SEQ ID NO: 886)
Cys Cys Glu Met Cys Cys Asn Pro Ala Cys Thr
Gly Cys Tyr
(SEQ ID NO: 887)
Cys Cys Glu Phe Cys Cys Asn Pro Ala Cys Thr
Gly Cys Tyr
(SEQ ID NO: 888)
Cys Cys Glu Pro Cys Cys Asn Pro Ala Cys Thr
Gly Cys Tyr
(SEQ ID NO: 889)
Cys Cys Glu Ser Cys Cys Asn Pro Ala Cys Thr
Gly Cys Tyr
(SEQ ID NO: 890)
Cys Cys Glu Thr Cys Cys Asn Pro Ala Cys Thr
Gly Cys Tyr
(SEQ ID NO: 891)
Cys Cys Glu Trp Cys Cys Asn Pro Ala Cys Thr
Gly Cys Tyr
(SEQ ID NO: 892)
Cys Cys Glu Val Cys Cys Asn Pro Ala Cys Thr
Gly Cys Tyr
(SEQ ID NO: 893)
Cys Cys Glu Tyr Cys Cys Asn Pro Ala Cys Thr
Gly Cys
(SEQ ID NO: 894)
Cys Cys Glu Ala Cys Cys Asn Pro Ala Cys Thr
Gly Cys
(SEQ ID NO: 895)
Cys Cys Glu Arg Cys Cys Asn Pro Ala Cys Thr
Gly Cys
(SEQ ID NO: 896)
Cys Cys Glu Asn Cys Cys Asn Pro Ala Cys Thr
Gly Cys
(SEQ ID NO: 897)
Cys Cys Glu Asp Cys Cys Asn Pro Ala Cys Thr
Gly Cys
(SEQ ID NO: 898)
Cys Cys Glu Cys Cys Cys Asn Pro Ala Cys Thr
Gly Cys
(SEQ ID NO: 899)
Cys Cys Glu Gln Cys Cys Asn Pro Ala Cys Thr
Gly Cys
(SEQ ID NO: 900)
Cys Cys Glu Glu Cys Cys Asn Pro Ala Cys Thr
Gly Cys
(SEQ ID NO: 901)
Cys Cys Glu Gly Cys Cys Asn Pro Ala Cys Thr
Gly Cys
(SEQ ID NO: 902)
Cys Cys Glu His Cys Cys Asn Pro Ala Cys Thr
Gly Cys
(SEQ ID NO: 903)
Cys Cys Glu Ile Cys Cys Asn Pro Ala Cys Thr
Gly Cys
(SEQ ID NO: 904)
Cys Cys Glu Lys Cys Cys Asn Pro Ala Cys Thr
Gly Cys
(SEQ ID NO: 905)
Cys Cys Glu Met Cys Cys Asn Pro Ala Cys Thr
Gly Cys
(SEQ ID NO: 906)
Cys Cys Glu Phe Cys Cys Asn Pro Ala Cys Thr
Gly Cys
(SEQ ID NO: 907)
Cys Cys Glu Pro Cys Cys Asn Pro Ala Cys Thr
Gly Cys
(SEQ ID NO: 908)
Cys Cys Glu Ser Cys Cys Asn Pro Ala Cys Thr
Gly Cys
(SEQ ID NO: 909)
Cys Cys Glu Thr Cys Cys Asn Pro Ala Cys Thr
Gly Cys;
(SEQ ID NO: 910)
Cys Cys Glu Trp Cys Cys Asn Pro Ala Cys Thr
Gly Cys
(SEQ ID NO: 847)
Cys Cys Glu Val Cys Cys Asn Pro Ala Cys Thr
Gly Cys.

Also useful are peptides wherein D′ comprises the, consists of or consists essentially of any of the following sequences (SEQ ID NOs: 911-1422, respectively, as shown below):

(SEQ ID NO: 911
Cys Cys Glu Leu Cys Cys Ala Pro Ala Cys Thr
Gly Cys Tyr
(SEQ ID NO: 912
Cys Cys Glu Leu Cys Cys Val Pro Ala Cys Thr
Gly Cys Tyr
(SEQ ID NO: 913
Cys Cys Glu Leu Cys Cys Leu Pro Ala Cys Thr
Gly Cys Tyr
(SEQ ID NO: 914
Cys Cys Glu Leu Cys Cys Ile Pro Ala Cys Thr
Gly Cys Tyr
(SEQ ID NO: 915
Cys Cys Glu Leu Cys Cys Pro Pro Ala Cys Thr
Gly Cys Tyr
(SEQ ID NO: 916
Cys Cys Glu Leu Cys Cys Met Pro Ala Cys Thr
Gly Cys Tyr
(SEQ ID NO: 917
Cys Cys Glu Leu Cys Cys Phe Pro Ala Cys Thr
Gly Cys Tyr
(SEQ ID NO: 918
Cys Cys Glu Leu Cys Cys Trp Pro Ala Cys Thr
Gly Cys Tyr
(SEQ ID NO: 919
Cys Cys Glu Leu Cys Cys Gly Pro Ala Cys Thr
Gly Cys Tyr
(SEQ ID NO: 920
Cys Cys Glu Leu Cys Cys Ser Pro Ala Cys Thr
Gly Cys Tyr
(SEQ ID NO: 921
Cys Cys Glu Leu Cys Cys Thr Pro Ala Cys Thr
Gly Cys Tyr
(SEQ ID NO: 922
Cys Cys Glu Leu Cys Cys Cys Pro Ala Cys Thr
Gly Cys Tyr
(SEQ ID NO: 923
Cys Cys Glu Leu Cys Cys Gln Pro Ala Cys Thr
Gly Cys Tyr
(SEQ ID NO: 924
Cys Cys Glu Leu Cys Cys Tyr Pro Ala Cys Thr
Gly Cys Tyr
(SEQ ID NO: 925
Cys Cys Glu Leu Cys Cys Asp Pro Ala Cys Thr
Gly Cys Tyr
(SEQ ID NO: 926
Cys Cys Glu Leu Cys Cys Glu Pro Ala Cys Thr
Gly Cys Tyr
(SEQ ID NO: 927
Cys Cys Glu Leu Cys Cys Lys Pro Ala Cys Thr
Gly Cys Tyr
(SEQ ID NO: 928
Cys Cys Glu Leu Cys Cys Arg Pro Ala Cys Thr
Gly Cys Tyr
(SEQ ID NO: 929
Cys Cys Glu Leu Cys Cys His Pro Ala Cys Thr
Gly Cys Tyr
(SEQ ID NO: 930
Cys Cys Glu Tyr Cys Cys Ala Pro Ala Cys Thr
Gly Cys Tyr
(SEQ ID NO: 931
Cys Cys Glu Tyr Cys Cys Val Pro Ala Cys Thr
Gly Cys Tyr
(SEQ ID NO: 932
Cys Cys Glu Tyr Cys Cys Leu Pro Ala Cys Thr
Gly Cys Tyr
(SEQ ID NO: 933
Cys Cys Glu Tyr Cys Cys Ile Pro Ala Cys Thr
Gly Cys Tyr
(SEQ ID NO: 934
Cys Cys Glu Tyr Cys Cys Pro Pro Ala Cys Thr
Gly Cys Tyr
(SEQ ID NO: 935
Cys Cys Glu Tyr Cys Cys Met Pro Ala Cys Thr
Gly Cys Tyr
(SEQ ID NO: 936
Cys Cys Glu Tyr Cys Cys Phe Pro Ala Cys Thr
Gly Cys Tyr
(SEQ ID NO: 937
Cys Cys Glu Tyr Cys Cys Trp Pro Ala Cys Thr
Gly Cys Tyr
(SEQ ID NO: 938
Cys Cys Glu Tyr Cys Cys Gly Pro Ala Cys Thr
Gly Cys Tyr
(SEQ ID NO: 939
Cys Cys Glu Tyr Cys Cys Ser Pro Ala Cys Thr
Gly Cys Tyr
(SEQ ID NO: 940
Cys Cys Glu Tyr Cys Cys Thr Pro Ala Cys Thr
Gly Cys Tyr
(SEQ ID NO: 941
Cys Cys Glu Tyr Cys Cys Cys Pro Ala Cys Thr
Gly Cys Tyr
(SEQ ID NO: 942
Cys Cys Glu Tyr Cys Cys Gln Pro Ala Cys Thr
Gly Cys Tyr
(SEQ ID NO: 943
Cys Cys Glu Tyr Cys Cys Tyr Pro Ala Cys Thr
Gly Cys Tyr
(SEQ ID NO: 944
Cys Cys Glu Tyr Cys Cys Asp Pro Ala Cys Thr
Gly Cys Tyr
(SEQ ID NO: 945
Cys Cys Glu Tyr Cys Cys Glu Pro Ala Cys Thr
Gly Cys Tyr
(SEQ ID NO: 946
Cys Cys Glu Tyr Cys Cys Lys Pro Ala Cys Thr
Gly Cys Tyr
(SEQ ID NO: 947
Cys Cys Glu Tyr Cys Cys Arg Pro Ala Cys Thr
Gly Cys Tyr
(SEQ ID NO: 948
Cys Cys Glu Tyr Cys Cys His Pro Ala Cys Thr
Gly Cys Tyr
(SEQ ID NO: 949
Cys Cys Glu Leu Cys Cys Ala Pro Ala Cys Thr
Gly Cys
(SEQ ID NO: 950
Cys Cys Glu Leu Cys Cys Val Pro Ala Cys Thr
Gly Cys
(SEQ ID NO: 951
Cys Cys Glu Leu Cys Cys Leu Pro Ala Cys Thr
Gly Cys
(SEQ ID NO: 952
Cys Cys Glu Leu Cys Cys Ile Pro Ala Cys Thr
Gly Cys
(SEQ ID NO: 953
Cys Cys Glu Leu Cys Cys Pro Pro Ala Cys Thr
Gly Cys
(SEQ ID NO: 954
Cys Cys Glu Leu Cys Cys Met Pro Ala Cys Thr
Gly Cys
(SEQ ID NO: 955
Cys Cys Glu Leu Cys Cys Phe Pro Ala Cys Thr
Gly Cys
(SEQ ID NO: 956
Cys Cys Glu Leu Cys Cys Trp Pro Ala Cys Thr
Gly Cys
(SEQ ID NO: 957
Cys Cys Glu Leu Cys Cys Gly Pro Ala Cys Thr
Gly Cys
(SEQ ID NO: 958
Cys Cys Glu Leu Cys Cys Ser Pro Ala Cys Thr
Gly Cys
(SEQ ID NO: 959
Cys Cys Glu Leu Cys Cys Thr Pro Ala Cys Thr
Gly Cys
(SEQ ID NO: 960
Cys Cys Glu Leu Cys Cys Cys Pro Ala Cys Thr
Gly Cys
(SEQ ID NO: 961
Cys Cys Glu Leu Cys Cys Gln Pro Ala Cys Thr
Gly Cys
(SEQ ID NO: 962
Cys Cys Glu Leu Cys Cys Tyr Pro Ala Cys Thr
Gly Cys
(SEQ ID NO: 963
Cys Cys Glu Leu Cys Cys Asp Pro Ala Cys Thr
Gly Cys
(SEQ ID NO: 964
Cys Cys Glu Leu Cys Cys Glu Pro Ala Cys Thr
Gly Cys
(SEQ ID NO: 965
Cys Cys Glu Leu Cys Cys Lys Pro Ala Cys Thr
Gly Cys
(SEQ ID NO: 966
Cys Cys Glu Leu Cys Cys Arg Pro Ala Cys Thr
Gly Cys
(SEQ ID NO: 967
Cys Cys Glu Leu Cys Cys His Pro Ala Cys Thr
Gly Cys
(SEQ ID NO: 968
Cys Cys Glu Tyr Cys Cys Ala Pro Ala Cys Thr
Gly Cys
(SEQ ID NO: 969
Cys Cys Glu Tyr Cys Cys Val Pro Ala Cys Thr
Gly Cys
(SEQ ID NO: 970
Cys Cys Glu Tyr Cys Cys Leu Pro Ala Cys Thr
Gly Cys
(SEQ ID NO: 971
Cys Cys Glu Tyr Cys Cys Ile Pro Ala Cys Thr
Gly Cys
(SEQ ID NO: 972
Cys Cys Glu Tyr Cys Cys Pro Pro Ala Cys Thr
Gly Cys
(SEQ ID NO: 973
Cys Cys Glu Tyr Cys Cys Met Pro Ala Cys Thr
Gly Cys
(SEQ ID NO: 974
Cys Cys Glu Tyr Cys Cys Phe Pro Ala Cys Thr
Gly Cys
(SEQ ID NO: 975
Cys Cys Glu Tyr Cys Cys Trp Pro Ala Cys Thr
Gly Cys
(SEQ ID NO: 976
Cys Cys Glu Tyr Cys Cys Gly Pro Ala Cys Thr
Gly Cys
(SEQ ID NO: 977
Cys Cys Glu Tyr Cys Cys Ser Pro Ala Cys Thr
Gly Cys
(SEQ ID NO: 978
Cys Cys Glu Tyr Cys Cys Thr Pro Ala Cys Thr
Gly Cys
(SEQ ID NO: 979
Cys Cys Glu Tyr Cys Cys Cys Pro Ala Cys Thr
Gly Cys
(SEQ ID NO: 980
Cys Cys Glu Tyr Cys Cys Gln Pro Ala Cys Thr
Gly Cys
(SEQ ID NO: 981
Cys Cys Glu Tyr Cys Cys Tyr Pro Ala Cys Thr
Gly Cys
(SEQ ID NO: 982
Cys Cys Glu Tyr Cys Cys Asp Pro Ala Cys Thr
Gly Cys
(SEQ ID NO: 983
Cys Cys Glu Tyr Cys Cys Glu Pro Ala Cys Thr
Gly Cys
(SEQ ID NO: 984
Cys Cys Glu Tyr Cys Cys Lys Pro Ala Cys Thr
Gly Cys
(SEQ ID NO: 985
Cys Cys Glu Tyr Cys Cys Arg Pro Ala Cys Thr
Gly Cys
(SEQ ID NO: 986
Cys Cys Glu Tyr Cys Cys His Pro Ala Cys Thr
Gly Cys
(SEQ ID NO: 987
Cys Cys Glu Leu Cys Cys Asn Pro Thr Cys Thr
Gly Cys Tyr
(SEQ ID NO: 988
Cys Cys Glu Tyr Cys Cys Asn Pro Thr Cys Thr
Gly Cys Tyr
(SEQ ID NO: 989
Cys Cys Glu Leu Cys Cys Asn Pro Thr Cys Thr
Gly Cys
(SEQ ID NO: 990
Cys Cys Glu Tyr Cys Cys Asn Pro Thr Cys Thr
Gly Cys
(SEQ ID NO: 991
Cys Cys Glu Phe Cys Cys Asn Pro Thr Cys Thr
Gly Cys Tyr
(SEQ ID NO: 992
Cys Cys Glu Phe Cys Cys Asn Pro Thr Cys Thr
Gly Cys
(SEQ ID NO: 993
Cys Cys Glu Trp Cys Cys Asn Pro Thr Cys Thr
Gly Cys Tyr
(SEQ ID NO: 994
Cys Cys Glu Trp Cys Cys Asn Pro Thr Cys Thr
Gly Cys
(SEQ ID NO: 995
Cys Cys Glu Leu Cys Cys Asn Gly Ala Cys Thr
Gly Cys Tyr
(SEQ ID NO: 996
Cys Cys Glu Tyr Cys Cys Asn Gly Ala Cys Thr
Gly Cys Tyr
(SEQ ID NO: 997
Cys Cys Glu Leu Cys Cys Asn Gly Ala Cys Thr
Gly Cys
(SEQ ID NO: 998
Cys Cys Glu Tyr Cys Cys Asn Gly Ala Cys Thr
Gly Cys
(SEQ ID NO: 999
Cys Cys Glu Phe Cys Cys Asn Gly Ala Cys Thr
Gly Cys Tyr
(SEQ ID NO: 1000
Cys Cys Glu Phe Cys Cys Asn Gly Ala Cys Thr
Gly Cys
(SEQ ID NO: 1001
Cys Cys Glu Trp Cys Cys Asn Gly Ala Cys Thr
Gly Cys Tyr
(SEQ ID NO: 1002
Cys Cys Glu Trp Cys Cys Asn Gly Ala Cys Thr
Gly Cys
(SEQ ID NO: 1003
Cys Cys Glu Leu Cys Cys Asn Pro Ala Cys Val
Gly Cys Tyr
(SEQ ID NO: 1004
Cys Cys Glu Tyr Cys Cys Asn Pro Ala Cys Val
Gly Cys Tyr
(SEQ ID NO: 1005
Cys Cys Glu Leu Cys Cys Asn Pro Ala Cys Val
Gly Cys
(SEQ ID NO: 1006
Cys Cys Glu Tyr Cys Cys Asn Pro Ala Cys Val
Gly Cys
(SEQ ID NO: 1007
Cys Cys Glu Phe Cys Cys Asn Pro Ala Cys Val
Gly Cys Tyr
(SEQ ID NO: 1008
Cys Cys Glu Phe Cys Cys Asn Pro Ala Cys Val
Gly Cys
(SEQ ID NO: 1009
Cys Cys Glu Trp Cys Cys Asn Pro Ala Cys Val
Gly Cys Tyr
(SEQ ID NO: 1010
Cys Cys Glu Trp Cys Cys Asn Pro Ala Cys Val
Gly Cys
(SEQ ID NO: 1011
Cys Cys Glu Leu Cys Cys Asn Pro Ala Cys Gly
Gly Cys Tyr
(SEQ ID NO: 1012
Cys Cys Glu Tyr Cys Cys Asn Pro Ala Cys Gly
Gly Cys Tyr
(SEQ ID NO: 1013
Cys Cys Glu Leu Cys Cys Asn Pro Ala Cys Gly
Gly Cys
(SEQ ID NO: 1014
Cys Cys Glu Tyr Cys Cys Asn Pro Ala Cys Gly
Gly Cys
(SEQ ID NO: 1015
Cys Cys Glu Phe Cys Cys Asn Pro Ala Cys Gly
Gly Cys Tyr
(SEQ ID NO: 1016
Cys Cys Glu Phe Cys Cys Asn Pro Ala Cys Gly
Gly Cys
(SEQ ID NO: 1017
Cys Cys Glu Trp Cys Cys Asn Pro Ala Cys Gly
Gly Cys Tyr
(SEQ ID NO: 1018
Cys Cys Glu Trp Cys Cys Asn Pro Ala Cys Gly
Gly Cys
(SEQ ID NO: 1019
Cys Cys Glu Leu Cys Cys Asn Pro Ala Cys Thr
Ala Cys Tyr
(SEQ ID NO: 1020
Cys Cys Glu Tyr Cys Cys Asn Pro Ala Cys Thr
Ala Cys Tyr
(SEQ ID NO: 1021
Cys Cys Glu Leu Cys Cys Asn Pro Ala Cys Thr
Ala Cys
(SEQ ID NO: 1022
Cys Cys Glu Tyr Cys Cys Asn Pro Ala Cys Thr
Ala Cys
(SEQ ID NO: 1023
Cys Cys Glu Trp Cys Cys Asn Pro Ala Cys Thr
Ala Cys Tyr
(SEQ ID NO: 1024
Cys Cys Glu Trp Cys Cys Asn Pro Ala Cys Thr
Ala Cys
(SEQ ID NO: 1025
Cys Cys Glu Phe Cys Cys Asn Pro Ala Cys Thr
Ala Cys Tyr
(SEQ ID NO: 1026
Cys Cys Glu Phe Cys Cys Asn Pro Ala Cys Thr
Ala Cys
(SEQ ID NO: 1027
Cys Cys Glu Leu Cys Cys Asn Pro Ala Cys Thr
Gly Cys Ala
(SEQ ID NO: 1028
Cys Cys Glu Leu Cys Cys Asn Pro Ala Cys Thr
Gly Cys Val
(SEQ ID NO: 1029
Cys Cys Glu Leu Cys Cys Asn Pro Ala Cys Thr
Gly Cys Leu
(SEQ ID NO: 1030
Cys Cys Glu Leu Cys Cys Asn Pro Ala Cys Thr
Gly Cys Ile
(SEQ ID NO: 1031
Cys Cys Glu Leu Cys Cys Asn Pro Ala Cys Thr
Gly Cys Pro
(SEQ ID NO: 1032
Cys Cys Glu Leu Cys Cys Asn Pro Ala Cys Thr
Gly Cys Met
(SEQ ID NO: 1033
Cys Cys Glu Leu Cys Cys Asn Pro Ala Cys Thr
Gly Cys Ph
(SEQ ID NO: 1034
Cys Cys Glu Leu Cys Cys Asn Pro Ala Cys Thr
Gly Cys Trp
(SEQ ID NO: 1035
Cys Cys Glu Leu Cys Cys Asn Pro Ala Cys Thr
Gly Cys Gly
(SEQ ID NO: 1036
Cys Cys Glu Leu Cys Cys Asn Pro Ala Cys Thr
Gly Cys Ser
(SEQ ID NO: 1037
Cys Cys Glu Leu Cys Cys Asn Pro Ala Cys Thr
Gly Cys Thr
(SEQ ID NO: 1038
Cys Cys Glu Leu Cys Cys Asn Pro Ala Cys Thr
Gly Cys Cys
(SEQ ID NO: 1039
Cys Cys Glu Leu Cys Cys Asn Pro Ala Cys Thr
Gly Cys Asn
(SEQ ID NO: 1040
Cys Cys Glu Leu Cys Cys Asn Pro Ala Cys Thr
Gly Cys Gln
(SEQ ID NO: 1041
Cys Cys Glu Leu Cys Cys Asn Pro Ala Cys Thr
Gly Cys Asp
(SEQ ID NO: 1042
Cys Cys Glu Leu Cys Cys Asn Pro Ala Cys Thr
Gly Cys Glu
(SEQ ID NO: 1043
Cys Cys Glu Leu Cys Cys Asn Pro Ala Cys Thr
Gly Cys Lys
(SEQ ID NO: 1044
Cys Cys Glu Leu Cys Cys Asn Pro Ala Cys Thr
Gly Cys Arg
(SEQ ID NO: 1045
Cys Cys Glu Leu Cys Cys Asn Pro Ala Cys Thr
Gly Cys His
(SEQ ID NO: 1046
Cys Cys Glu Tyr Cys Cys Asn Pro Ala Cys Thr
Gly Cys Ala
(SEQ ID NO: 1047
Cys Cys Glu Tyr Cys Cys Asn Pro Ala Cys Thr
Gly Cys Val
(SEQ ID NO: 1048
Cys Cys Glu Tyr Cys Cys Asn Pro Ala Cys Thr
Gly Cys Leu
(SEQ ID NO: 1049
Cys Cys Glu Tyr Cys Cys Asn Pro Ala Cys Thr
Gly Cys Ile
(SEQ ID NO: 1050
Cys Cys Glu Tyr Cys Cys Asn Pro Ala Cys Thr
Gly Cys Pro
(SEQ ID NO: 1051
Cys Cys Glu Tyr Cys Cys Asn Pro Ala Cys Thr
Gly Cys Met
(SEQ ID NO: 1052
Cys Cys Glu Tyr Cys Cys Asn Pro Ala Cys Thr
Gly Cys Phe
(SEQ ID NO: 1053
Cys Cys Glu Tyr Cys Cys Asn Pro Ala Cys Thr
Gly Cys Trp
(SEQ ID NO: 1054
Cys Cys Glu Tyr Cys Cys Asn Pro Ala Cys Thr
Gly Cys Gly
(SEQ ID NO: 1055
Cys Cys Glu Tyr Cys Cys Asn Pro Ala Cys Thr
Gly Cys Ser
(SEQ ID NO: 1056
Cys Cys Glu Tyr Cys Cys Asn Pro Ala Cys Thr
Gly Cys Thr
(SEQ ID NO: 1057
Cys Cys Glu Tyr Cys Cys Asn Pro Ala Cys Thr
Gly Cys Cys
(SEQ ID NO: 1058
Cys Cys Glu Tyr Cys Cys Asn Pro Ala Cys Thr
Gly Cys Asn
(SEQ ID NO: 1059
Cys Cys Glu Tyr Cys Cys Asn Pro Ala Cys Thr
Gly Cys Gln
(SEQ ID NO: 1060
Cys Cys Glu Tyr Cys Cys Asn Pro Ala Cys Thr
Gly Cys Asp
(SEQ ID NO: 1061
Cys Cys Glu Tyr Cys Cys Asn Pro Ala Cys Thr
Gly Cys Glu
(SEQ ID NO: 1062
Cys Cys Glu Tyr Cys Cys Asn Pro Ala Cys Thr
Gly Cys Lys
(SEQ ID NO: 1063
Cys Cys Glu Tyr Cys Cys Asn Pro Ala Cys Thr
Gly Cys Arg
(SEQ ID NO: 1064
Cys Cys Glu Tyr Cys Cys Asn Pro Ala Cys Thr
Gly Cys His
(SEQ ID NO: 1065
Cys Cys Ala Leu Cys Cys Asn Pro Ala Cys Thr
Gly Cys Tyr
(SEQ ID NO: 1066
Cys Cys Val Leu Cys Cys Asn Pro Ala Cys Thr
Gly Cys Tyr
(SEQ ID NO: 1067
Cys Cys Leu Leu Cys Cys Asn Pro Ala Cys Thr
Gly Cys Tyr
(SEQ ID NO: 1068
Cys Cys Ile Leu Cys Cys Asn Pro Ala Cys Thr
Gly Cys Tyr
(SEQ ID NO: 1069
Cys Cys Met Leu Cys Cys Asn Pro Ala Cys Thr
Gly Cys Tyr
(SEQ ID NO: 1070
Cys Cys Phe Leu Cys Cys Asn Pro Ala Cys Thr
Gly Cys Tyr
(SEQ ID NO: 1071
Cys Cys Trp Leu Cys Cys Asn Pro Ala Cys Thr
Gly Cys Tyr
(SEQ ID NO: 1072
Cys Cys Gly Leu Cys Cys Asn Pro Ala Cys Thr
Gly Cys Tyr
(SEQ ID NO: 1073
Cys Cys Ser Leu Cys Cys Asn Pro Ala Cys Thr
Gly Cys Tyr
(SEQ ID NO: 1074
Cys Cys Thr Leu Cys Cys Asn Pro Ala Cys Thr
Gly Cys Tyr
(SEQ ID NO: 1075
Cys Cys Cys Leu Cys Cys Asn Pro Ala Cys Thr
Gly Cys Tyr
(SEQ ID NO: 1076
Cys Cys Asn Leu Cys Cys Asn Pro Ala Cys Thr
Gly Cys Tyr
(SEQ ID NO: 1077
Cys Cys Gln Leu Cys Cys Asn Pro Ala Cys Thr
Gly Cys Tyr
(SEQ ID NO: 1078
Cys Cys Tyr Leu Cys Cys Asn Pro Ala Cys Thr
Gly Cys Tyr
(SEQ ID NO: 1079
Cys Cys Asp Leu Cys Cys Asn Pro Ala Cys Thr
Gly Cys Tyr
(SEQ ID NO: 1080
Cys Cys Lys Leu Cys Cys Asn Pro Ala Cys Thr
Gly Cys Tyr
(SEQ ID NO: 1081
Cys Cys Arg Leu Cys Cys Asn Pro Ala Cys Thr
Gly Cys Tyr
(SEQ ID NO: 1082
Cys Cys His Leu Cys Cys Asn Pro Ala Cys Thr
Gly Cys Tyr
(SEQ ID NO: 1083
Cys Cys Ala Leu Cys Cys Asn Pro Ala Cys Thr
Gly Cys
(SEQ ID NO: 1084
Cys Cys Val Leu Cys Cys Asn Pro Ala Cys Thr
Gly Cys
(SEQ ID NO: 1085
Cys Cys Leu Leu Cys Cys Asn Pro Ala Cys Thr
Gly Cys
(SEQ ID NO: 1086
Cys Cys Ile Leu Cys Cys Asn Pro Ala Cys Thr
Gly Cys
(SEQ ID NO: 1087
Cys Cys Met Leu Cys Cys Asn Pro Ala Cys Thr
Gly Cys
(SEQ ID NO: 1088
Cys Cys Phe Leu Cys Cys Asn Pro Ala Cys Thr
Gly Cys
(SEQ ID NO: 1089
Cys Cys Trp Leu Cys Cys Asn Pro Ala Cys Thr
Gly Cys
(SEQ ID NO: 1090
Cys Cys Gly Leu Cys Cys Asn Pro Ala Cys Thr
Gly Cys
(SEQ ID NO: 1091
Cys Cys Ser Leu Cys Cys Asn Pro Ala Cys Thr
Gly Cys
(SEQ ID NO: 1092
Cys Cys Thr Leu Cys Cys Asn Pro Ala Cys Thr
Gly Cys
(SEQ ID NO: 1093
Cys Cys Cys Leu Cys Cys Asn Pro Ala Cys Thr
Gly Cys
(SEQ ID NO: 1094
Cys Cys Asn Leu Cys Cys Asn Pro Ala Cys Thr
Gly Cys
(SEQ ID NO: 1095
Cys Cys Gln Leu Cys Cys Asn Pro Ala Cys Thr
Gly Cys
(SEQ ID NO: 1096
Cys Cys Tyr Leu Cys Cys Asn Pro Ala Cys Thr
Gly Cys
(SEQ ID NO: 1097
Cys Cys Asp Leu Cys Cys Asn Pro Ala Cys Thr
Gly Cys
(SEQ ID NO: 1098
Cys Cys Lys Leu Cys Cys Asn Pro Ala Cys Thr
Gly Cys
(SEQ ID NO: 1099
Cys Cys Arg Leu Cys Cys Asn Pro Ala Cys Thr
Gly Cys
(SEQ ID NO: 1100
Cys Cys His Leu Cys Cys Asn Pro Ala Cys Thr
Gly Cys
(SEQ ID NO: 1101
Cys Cys Ala Tyr Cys Cys Asn Pro Ala Cys Thr
Gly Cys Tyr
(SEQ ID NO: 1102
Cys Cys Val Tyr Cys Cys Asn Pro Ala Cys Thr
Gly Cys Tyr
(SEQ ID NO: 1103
Cys Cys Leu Tyr Cys Cys Asn Pro Ala Cys Thr
Gly Cys Tyr
(SEQ ID NO: 1104
Cys Cys Ile Tyr Cys Cys Asn Pro Ala Cys Thr
Gly Cys Tyr
(SEQ ID NO: 1105
Cys Cys Met Tyr Cys Cys Asn Pro Ala Cys Thr
Gly Cys Tyr
(SEQ ID NO: 1106
Cys Cys Phe Tyr Cys Cys Asn Pro Ala Cys Thr
Gly Cys Tyr
(SEQ ID NO: 1107
Cys Cys Trp Tyr Cys Cys Asn Pro Ala Cys Thr
Gly Cys Tyr
(SEQ ID NO: 1108
Cys Cys Gly Tyr Cys Cys Asn Pro Ala Cys Thr
Gly Cys Tyr
(SEQ ID NO: 1109
Cys Cys Ser Tyr Cys Cys Asn Pro Ala Cys Thr
Gly Cys Tyr
(SEQ ID NO: 1110
Cys Cys Thr Tyr Cys Cys Asn Pro Ala Cys Thr
Gly Cys Tyr
(SEQ ID NO: 1111
Cys Cys Cys Tyr Cys Cys Asn Pro Ala Cys Thr
Gly Cys Tyr
(SEQ ID NO: 1112
Cys Cys Asn Tyr Cys Cys Asn Pro Ala Cys Thr
Gly Cys Tyr
(SEQ ID NO: 1113
Cys Cys Gln Tyr Cys Cys Asn Pro Ala Cys Thr
Gly Cys Tyr
(SEQ ID NO: 1114
Cys Cys Tyr Tyr Cys Cys Asn Pro Ala Cys Thr
Gly Cys Tyr
(SEQ ID NO: 1115
Cys Cys Asp Tyr Cys Cys Asn Pro Ala Cys Thr
Gly Cys Tyr
(SEQ ID NO: 1116
Cys Cys Lys Tyr Cys Cys Asn Pro Ala Cys Thr
Gly Cys Tyr
(SEQ ID NO: 1117
Cys Cys Arg Tyr Cys Cys Asn Pro Ala Cys Thr
Gly Cys Tyr
(SEQ ID NO: 1118
Cys Cys His Tyr Cys Cys Asn Pro Ala Cys Thr
Gly Cys Tyr
(SEQ ID NO: 1119
Cys Cys Ala Tyr Cys Cys Asn Pro Ala Cys Thr
Gly Cys
(SEQ ID NO: 1120
Cys Cys Val Tyr Cys Cys Asn Pro Ala Cys Thr
Gly Cys
(SEQ ID NO: 1121
Cys Cys Leu Tyr Cys Cys Asn Pro Ala Cys Thr
Gly Cys
(SEQ ID NO: 1122
Cys Cys Ile Tyr Cys Cys Asn Pro Ala Cys Thr
Gly Cys
(SEQ ID NO: 1123
Cys Cys Met Tyr Cys Cys Asn Pro Ala Cys Thr
Gly Cys
(SEQ ID NO: 1124
Cys Cys Phe Tyr Cys Cys Asn Pro Ala Cys Thr
Gly Cys
(SEQ ID NO: 1125
Cys Cys Trp Tyr Cys Cys Asn Pro Ala Cys Thr
Gly Cys
(SEQ ID NO: 1126
Cys Cys Gly Tyr Cys Cys Asn Pro Ala Cys Thr
Gly Cys
(SEQ ID NO: 1127
Cys Cys Ser Tyr Cys Cys Asn Pro Ala Cys Thr
Gly Cys
(SEQ ID NO: 1128
Cys Cys Thr Tyr Cys Cys Asn Pro Ala Cys Thr
Gly Cys
(SEQ ID NO: 1129
Cys Cys Cys Tyr Cys Cys Asn Pro Ala Cys Thr
Gly Cys
(SEQ ID NO: 1130
Cys Cys Asn Tyr Cys Cys Asn Pro Ala Cys Thr
Gly Cys
(SEQ ID NO: 1131
Cys Cys Gln Tyr Cys Cys Asn Pro Ala Cys Thr
Gly Cys
(SEQ ID NO: 1132
Cys Cys Tyr Tyr Cys Cys Asn Pro Ala Cys Thr
Gly Cys
(SEQ ID NO: 1133
Cys Cys Asp Tyr Cys Cys Asn Pro Ala Cys Thr
Gly Cys
(SEQ ID NO: 1134
Cys Cys Lys Tyr Cys Cys Asn Pro Ala Cys Thr
Gly Cys
(SEQ ID NO: 1135
Cys Cys Arg Tyr Cys Cys Asn Pro Ala Cys Thr
Gly Cys
(SEQ ID NO: 1136
Cys Cys His Tyr Cys Cys Asn Pro Ala Cys Thr
Gly Cys
(SEQ ID NO: 1137
Cys Cys Glu Phe Cys Cys Ala Pro Ala Cys Thr
Gly Cys Tyr
(SEQ ID NO: 1138
Cys Cys Glu Phe Cys Cys Val Pro Ala Cys Thr
Gly Cys Tyr
(SEQ ID NO: 1139
Cys Cys Glu Phe Cys Cys Leu Pro Ala Cys Thr
Gly Cys Tyr
(SEQ ID NO: 1140
Cys Cys Glu Phe Cys Cys Ile Pro Ala Cys Thr
Gly Cys Tyr
(SEQ ID NO: 1141
Cys Cys Glu Phe Cys Cys Pro Pro Ala Cys Thr
Gly Cys Tyr
(SEQ ID NO: 1142
Cys Cys Glu Phe Cys Cys Met Pro Ala Cys Thr
Gly Cys Tyr
(SEQ ID NO: 1143
Cys Cys Glu Phe Cys Cys Phe Pro Ala Cys Thr
Gly Cys Tyr
(SEQ ID NO: 1144
Cys Cys Glu Phe Cys Cys Trp Pro Ala Cys Thr
Gly Cys Tyr
(SEQ ID NO: 1145
Cys Cys Glu Phe Cys Cys Gly Pro Ala Cys Thr
Gly Cys Tyr
(SEQ ID NO: 1146
Cys Cys Glu Phe Cys Cys Ser Pro Ala Cys Thr
Gly Cys Tyr
(SEQ ID NO: 1147
Cys Cys Glu Phe Cys Cys Thr Pro Ala Cys Thr
Gly Cys Tyr
(SEQ ID NO: 1148
Cys Cys Glu Phe Cys Cys Cys Pro Ala Cys Thr
Gly Cys Tyr
(SEQ ID NO: 1149
Cys Cys Glu Phe Cys Cys Gln Pro Ala Cys Thr
Gly Cys Tyr
(SEQ ID NO: 1150
Cys Cys Glu Phe Cys Cys Tyr Pro Ala Cys Thr
Gly Cys Tyr
(SEQ ID NO: 1151
Cys Cys Glu Phe Cys Cys Asp Pro Ala Cys Thr
Gly Cys Tyr
(SEQ ID NO: 1152
Cys Cys Glu Phe Cys Cys Glu Pro Ala Cys Thr
Gly Cys Tyr
(SEQ ID NO: 1153
Cys Cys Glu Phe Cys Cys Lys Pro Ala Cys Thr
Gly Cys Tyr
(SEQ ID NO: 1154
Cys Cys Glu Phe Cys Cys Arg Pro Ala Cys Thr
Gly Cys Tyr
(SEQ ID NO: 1155
Cys Cys Glu Phe Cys Cys His Pro Ala Cys Thr
Gly Cys Tyr
(SEQ ID NO: 1156
Cys Cys Glu Phe Cys Cys Ala Pro Ala Cys Thr
Gly Cys
(SEQ ID NO: 1157
Cys Cys Glu Phe Cys Cys Val Pro Ala Cys Thr
Gly Cys
(SEQ ID NO: 1158
Cys Cys Glu Phe Cys Cys Leu Pro Ala Cys Thr
Gly Cys
(SEQ ID NO: 1159
Cys Cys Glu Phe Cys Cys Ile Pro Ala Cys Thr
Gly Cys
(SEQ ID NO: 1160
Cys Cys Glu Phe Cys Cys Pro Pro Ala Cys Thr
Gly Cys
(SEQ ID NO: 1161
Cys Cys Glu Phe Cys Cys Met Pro Ala Cys Thr
Gly Cys
(SEQ ID NO: 1162
Cys Cys Glu Phe Cys Cys Phe Pro Ala Cys Thr
Gly Cys
(SEQ ID NO: 1163
Cys Cys Glu Phe Cys Cys Trp Pro Ala Cys Thr
Gly Cys
(SEQ ID NO: 1164
Cys Cys Glu Phe Cys Cys Gly Pro Ala Cys Thr
Gly Cys
(SEQ ID NO: 1165
Cys Cys Glu Phe Cys Cys Ser Pro Ala Cys Thr
Gly Cys
(SEQ ID NO: 1166
Cys Cys Glu Phe Cys Cys Thr Pro Ala Cys Thr
Gly Cys
(SEQ ID NO: 1167
Cys Cys Glu Phe Cys Cys Cys Pro Ala Cys Thr
Gly Cys
(SEQ ID NO: 1168
Cys Cys Glu Phe Cys Cys Gln Pro Ala Cys Thr
Gly Cys
(SEQ ID NO: 1169
Cys Cys Glu Phe Cys Cys Tyr Pro Ala Cys Thr
Gly Cys
(SEQ ID NO: 1170
Cys Cys Glu Phe Cys Cys Asp Pro Ala Cys Thr
Gly Cys
(SEQ ID NO: 1171
Cys Cys Glu Phe Cys Cys Glu Pro Ala Cys Thr
Gly Cys
(SEQ ID NO: 1172
Cys Cys Glu Phe Cys Cys Lys Pro Ala Cys Thr
Gly Cys
(SEQ ID NO: 1173
Cys Cys Glu Phe Cys Cys Arg Pro Ala Cys Thr
Gly Cys
(SEQ ID NO: 1174
Cys Cys Glu Phe Cys Cys His Pro Ala Cys Thr
Gly Cys
(SEQ ID NO: 1175
Cys Cys Glu Trp Cys Cys Ala Pro Ala Cys Thr
Gly Cys Tyr
(SEQ ID NO: 1176
Cys Cys Glu Trp Cys Cys Val Pro Ala Cys Thr
Gly Cys Tyr
(SEQ ID NO: 1177
Cys Cys Glu Trp Cys Cys Leu Pro Ala Cys Thr
Gly Cys Tyr
(SEQ ID NO: 1178
Cys Cys Glu Trp Cys Cys Ile Pro Ala Cys Thr
Gly Cys Tyr
(SEQ ID NO: 1179
Cys Cys Glu Trp Cys Cys Pro Pro Ala Cys Thr
Gly Cys Tyr
(SEQ ID NO: 1180
Cys Cys Glu Trp Cys Cys Met Pro Ala Cys Thr
Gly Cys Tyr
(SEQ ID NO: 1181
Cys Cys Glu Trp Cys Cys Phe Pro Ala Cys Thr
Gly Cys Tyr
(SEQ ID NO: 1182
Cys Cys Glu Trp Cys Cys Trp Pro Ala Cys Thr
Gly Cys Tyr
(SEQ ID NO: 1183
Cys Cys Glu Trp Cys Cys Gly Pro Ala Cys Thr
Gly Cys Tyr
(SEQ ID NO: 1184
Cys Cys Glu Trp Cys Cys Ser Pro Ala Cys Thr
Gly Cys Tyr
(SEQ ID NO: 1185
Cys Cys Glu Trp Cys Cys Thr Pro Ala Cys Thr
Gly Cys Tyr
(SEQ ID NO: 1186
Cys Cys Glu Trp Cys Cys Cys Pro Ala Cys Thr
Gly Cys Tyr
(SEQ ID NO: 1187
Cys Cys Glu Trp Cys Cys Gln Pro Ala Cys Thr
Gly Cys Tyr
(SEQ ID NO: 1188
Cys Cys Glu Trp Cys Cys Tyr Pro Ala Cys Thr
Gly Cys Tyr
(SEQ ID NO: 1189
Cys Cys Glu Trp Cys Cys Asp Pro Ala Cys Thr
Gly Cys Tyr
(SEQ ID NO: 1190
Cys Cys Glu Trp Cys Cys Glu Pro Ala Cys Thr
Gly Cys Tyr
(SEQ ID NO: 1191
Cys Cys Glu Trp Cys Cys Lys Pro Ala Cys Thr
Gly Cys Tyr
(SEQ ID NO: 1192
Cys Cys Glu Trp Cys Cys Arg Pro Ala Cys Thr
Gly Cys Tyr
(SEQ ID NO: 1193
Cys Cys Glu Trp Cys Cys His Pro Ala Cys Thr
Gly Cys Tyr
(SEQ ID NO: 1194
Cys Cys Glu Trp Cys Cys Ala Pro Ala Cys Thr
Gly Cys
(SEQ ID NO: 1195
Cys Cys Glu Trp Cys Cys Val Pro Ala Cys Thr
Gly Cys
(SEQ ID NO: 1196
Cys Cys Glu Trp Cys Cys Leu Pro Ala Cys Thr
Gly Cys
(SEQ ID NO: 1197
Cys Cys Glu Trp Cys Cys Ile Pro Ala Cys Thr
Gly Cys
(SEQ ID NO: 1198
Cys Cys Glu Trp Cys Cys Pro Pro Ala Cys Thr
Gly Cys
(SEQ ID NO: 1199
Cys Cys Glu Trp Cys Cys Met Pro Ala Cys Thr
Gly Cys
(SEQ ID NO: 1200
Cys Cys Glu Trp Cys Cys Phe Pro Ala Cys Thr
Gly Cys
(SEQ ID NO: 1201
Cys Cys Glu Trp Cys Cys Trp Pro Ala Cys Thr
Gly Cys
(SEQ ID NO: 1202
Cys Cys Glu Trp Cys Cys Gly Pro Ala Cys Thr
Gly Cys
(SEQ ID NO: 1203
Cys Cys Glu Trp Cys Cys Ser Pro Ala Cys Thr
Gly Cys
(SEQ ID NO: 1204
Cys Cys Glu Trp Cys Cys Thr Pro Ala Cys Thr
Gly Cys
(SEQ ID NO: 1205
Cys Cys Glu Trp Cys Cys Cys Pro Ala Cys Thr
Gly Cys
(SEQ ID NO: 1206
Cys Cys Glu Trp Cys Cys Gln Pro Ala Cys Thr
Gly Cys
(SEQ ID NO: 1207
Cys Cys Glu Trp Cys Cys Tyr Pro Ala Cys Thr
Gly Cys
(SEQ ID NO: 1208
Cys Cys Glu Trp Cys Cys Asp Pro Ala Cys Thr
Gly Cys
(SEQ ID NO: 1209
Cys Cys Glu Trp Cys Cys Glu Pro Ala Cys Thr
Gly Cys
(SEQ ID NO: 1210
Cys Cys Glu Trp Cys Cys Lys Pro Ala Cys Thr
Gly Cys
(SEQ ID NO: 1211
Cys Cys Glu Trp Cys Cys Arg Pro Ala Cys Thr
Gly Cys
(SEQ ID NO: 1212
Cys Cys Glu Trp Cys Cys His Pro Ala Cys Thr
Gly Cys
(SEQ ID NO: 1213
Cys Cys Glu Trp Cys Cys Asn Pro Ala Cys Thr
Gly Cys Ala
(SEQ ID NO: 1214
Cys Cys Glu Trp Cys Cys Asn Pro Ala Cys Thr
Gly Cys Val
(SEQ ID NO: 1215
Cys Cys Glu Trp Cys Cys Asn Pro Ala Cys Thr
Gly Cys Leu
(SEQ ID NO: 1216
Cys Cys Glu Trp Cys Cys Asn Pro Ala Cys Thr
Gly Cys Ile
(SEQ ID NO: 1217
Cys Cys Glu Trp Cys Cys Asn Pro Ala Cys Thr
Gly Cys Pro
(SEQ ID NO: 1218
Cys Cys Glu Trp Cys Cys Asn Pro Ala Cys Thr
Gly Cys Met
(SEQ ID NO: 1219
Cys Cys Glu Trp Cys Cys Asn Pro Ala Cys Thr
Gly Cys Phe
(SEQ ID NO: 1220
Cys Cys Glu Trp Cys Cys Asn Pro Ala Cys Thr
Gly Cys Trp
(SEQ ID NO: 1221
Cys Cys Glu Trp Cys Cys Asn Pro Ala Cys Thr
Gly Cys Gly
(SEQ ID NO: 1222
Cys Cys Glu Trp Cys Cys Asn Pro Ala Cys Thr
Gly Cys Ser
(SEQ ID NO: 1223
Cys Cys Glu Trp Cys Cys Asn Pro Ala Cys Thr
Gly Cys Thr
(SEQ ID NO: 1224
Cys Cys Glu Trp Cys Cys Asn Pro Ala Cys Thr
Gly Cys Cys
(SEQ ID NO: 1225
Cys Cys Glu Trp Cys Cys Asn Pro Ala Cys Thr
Gly Cys Asn
(SEQ ID NO: 1226
Cys Cys Glu Trp Cys Cys Asn Pro Ala Cys Thr
Gly Cys Gln
(SEQ ID NO: 1227
Cys Cys Glu Trp Cys Cys Asn Pro Ala Cys Thr
Gly Cys Asp
(SEQ ID NO: 1228
Cys Cys Glu Trp Cys Cys Asn Pro Ala Cys Thr
Gly Cys Glu
(SEQ ID NO: 1229
Cys Cys Glu Trp Cys Cys Asn Pro Ala Cys Thr
Gly Cys Lys
(SEQ ID NO: 1230
Cys Cys Glu Trp Cys Cys Asn Pro Ala Cys Thr
Gly Cys Arg
(SEQ ID NO: 1231
Cys Cys Glu Trp Cys Cys Asn Pro Ala Cys Thr
Gly Cys His
(SEQ ID NO: 1232
Cys Cys Glu Phe Cys Cys Asn Pro Ala Cys Thr
Gly Cys Ala
(SEQ ID NO: 1233
Cys Cys Glu Phe Cys Cys Asn Pro Ala Cys Thr
Gly Cys Val
(SEQ ID NO: 1234
Cys Cys Glu Phe Cys Cys Asn Pro Ala Cys Thr
Gly Cys Leu
(SEQ ID NO: 1235
Cys Cys Glu Phe Cys Cys Asn Pro Ala Cys Thr
Gly Cys Ile
(SEQ ID NO: 1236
Cys Cys Glu Phe Cys Cys Asn Pro Ala Cys Thr
Gly Cys Pro
(SEQ ID NO: 1237
Cys Cys Glu Phe Cys Cys Asn Pro Ala Cys Thr
Gly Cys Met
(SEQ ID NO: 1238
Cys Cys Glu Phe Cys Cys Asn Pro Ala Cys Thr
Gly Cys Phe
(SEQ ID NO: 1239
Cys Cys Glu Phe Cys Cys Asn Pro Ala Cys Thr
Gly Cys Trp
(SEQ ID NO: 1240
Cys Cys Glu Phe Cys Cys Asn Pro Ala Cys Thr
Gly Cys Gly
(SEQ ID NO: 1241
Cys Cys Glu Phe Cys Cys Asn Pro Ala Cys Thr
Gly Cys Ser
(SEQ ID NO: 1242
Cys Cys Glu Phe Cys Cys Asn Pro Ala Cys Thr
Gly Cys Thr
(SEQ ID NO: 1243
Cys Cys Glu Phe Cys Cys Asn Pro Ala Cys Thr
Gly Cys Cys
(SEQ ID NO: 1244
Cys Cys Glu Phe Cys Cys Asn Pro Ala Cys Thr
Gly Cys Asn
(SEQ ID NO: 1245
Cys Cys Glu Phe Cys Cys Asn Pro Ala Cys Thr
Gly Cys Gln
(SEQ ID NO: 1246
Cys Cys Glu Phe Cys Cys Asn Pro Ala Cys Thr
Gly Cys Asp
(SEQ ID NO: 1247
Cys Cys Glu Phe Cys Cys Asn Pro Ala Cys Thr
Gly Cys Glu
(SEQ ID NO: 1248
Cys Cys Glu Phe Cys Cys Asn Pro Ala Cys Thr
Gly Cys Lys
(SEQ ID NO: 1249
Cys Cys Glu Phe Cys Cys Asn Pro Ala Cys Thr
Gly Cys Arg
(SEQ ID NO: 1250
Cys Cys Glu Phe Cys Cys Asn Pro Ala Cys Thr
Gly Cys His
(SEQ ID NO: 1251
Cys Cys Ala Phe Cys Cys Asn Pro Ala Cys Thr
Gly Cys Tyr
(SEQ ID NO: 1252
Cys Cys Val Phe Cys Cys Asn Pro Ala Cys Thr
Gly Cys Tyr
(SEQ ID NO: 1253
Cys Cys Leu Phe Cys Cys Asn Pro Ala Cys Thr
Gly Cys Tyr
(SEQ ID NO: 1254
Cys Cys Ile Phe Cys Cys Asn Pro Ala Cys Thr
Gly Cys Tyr
(SEQ ID NO: 1255
Cys Cys Met Phe Cys Cys Asn Pro Ala Cys Thr
Gly Cys Tyr
(SEQ ID NO: 1256
Cys Cys Phe Phe Cys Cys Asn Pro Ala Cys Thr
Gly Cys Tyr
(SEQ ID NO: 1257
Cys Cys Trp Phe Cys Cys Asn Pro Ala Cys Thr
Gly Cys Tyr
(SEQ ID NO: 1258
Cys Cys Gly Phe Cys Cys Asn Pro Ala Cys Thr
Gly Cys Tyr
(SEQ ID NO: 1259
Cys Cys Ser Phe Cys Cys Asn Pro Ala Cys Thr
Gly Cys Tyr
(SEQ ID NO: 1260
Cys Cys Thr Phe Cys Cys Asn Pro Ala Cys Thr
Gly Cys Tyr
(SEQ ID NO: 1261
Cys Cys Cys Phe Cys Cys Asn Pro Ala Cys Thr
Gly Cys Tyr
(SEQ ID NO: 1262
Cys Cys Asn Phe Cys Cys Asn Pro Ala Cys Thr
Gly Cys Tyr
(SEQ ID NO: 1263
Cys Cys Gln Phe Cys Cys Asn Pro Ala Cys Thr
Gly Cys Tyr
(SEQ ID NO: 1264
Cys Cys Tyr Phe Cys Cys Asn Pro Ala Cys Thr
Gly Cys Tyr
(SEQ ID NO: 1265
Cys Cys Asp Phe Cys Cys Asn Pro Ala Cys Thr
Gly Cys Tyr
(SEQ ID NO: 1266
Cys Cys Lys Phe Cys Cys Asn Pro Ala Cys Thr
Gly Cys Tyr
(SEQ ID NO: 1267
Cys Cys Arg Phe Cys Cys Asn Pro Ala Cys Thr
Gly Cys Tyr
(SEQ ID NO: 1268
Cys Cys His Phe Cys Cys Asn Pro Ala Cys Thr
Gly Cys Tyr
(SEQ ID NO: 1269
Cys Cys Ala Phe Cys Cys Asn Pro Ala Cys Thr
Gly Cys
(SEQ ID NO: 1270
Cys Cys Val Phe Cys Cys Asn Pro Ala Cys Thr
Gly Cys
(SEQ ID NO: 1271
Cys Cys Leu Phe Cys Cys Asn Pro Ala Cys Thr
Gly Cys
(SEQ ID NO: 1272
Cys Cys Ile Phe Cys Cys Asn Pro Ala Cys Thr
Gly Cys
(SEQ ID NO: 1273
Cys Cys Met Phe Cys Cys Asn Pro Ala Cys Thr
Gly Cys
(SEQ ID NO: 1274
Cys Cys Phe Phe Cys Cys Asn Pro Ala Cys Thr
Gly Cys
(SEQ ID NO: 1275
Cys Cys Trp Phe Cys Cys Asn Pro Ala Cys
Thr Gly Cys
(SEQ ID NO: 1276
Cys Cys Gly Phe Cys Cys Asn Pro Ala Cys Thr
Gly Cys
(SEQ ID NO: 1277
Cys Cys Ser Phe Cys Cys Asn Pro Ala Cys Thr
Gly Cys
(SEQ ID NO: 1278
Cys Cys Thr Phe Cys Cys Asn Pro Ala Cys Thr
Gly Cys
(SEQ ID NO: 1279
Cys Cys Cys Phe Cys Cys Asn Pro Ala Cys Thr
Gly Cys
(SEQ ID NO: 1280
Cys Cys Asn Phe Cys Cys Asn Pro Ala Cys Thr
Gly Cys
(SEQ ID NO: 1281
Cys Cys Gln Phe Cys Cys Asn Pro Ala Cys Thr
Gly Cys
(SEQ ID NO: 1282
Cys Cys Tyr Phe Cys Cys Asn Pro Ala Cys Thr
Gly Cys
(SEQ ID NO: 1283
Cys Cys Asp Phe Cys Cys Asn Pro Ala Cys Thr
Gly Cys
(SEQ ID NO: 1284
Cys Cys Lys Phe Cys Cys Asn Pro Ala Cys Thr
Gly Cys
(SEQ ID NO: 1285
Cys Cys Arg Phe Cys Cys Asn Pro Ala Cys Thr
Gly Cys
(SEQ ID NO: 1286
Cys Cys His Phe Cys Cys Asn Pro Ala Cys Thr
Gly Cys
(SEQ ID NO: 1287
Cys Cys Ala Trp Cys Cys Asn Pro Ala Cys Thr
Gly Cys Tyr
(SEQ ID NO: 1288
Cys Cys Val Trp Cys Cys Asn Pro Ala Cys Thr
Gly Cys Tyr
(SEQ ID NO: 1289
Cys Cys Leu Trp Cys Cys Asn Pro Ala Cys Thr
Gly Cys Tyr
(SEQ ID NO: 1290
Cys Cys Ile Trp Cys Cys Asn Pro Ala Cys Thr
Gly Cys Tyr
(SEQ ID NO: 1291
Cys Cys Met Trp Cys Cys Asn Pro Ala Cys Thr
Gly Cys Tyr
(SEQ ID NO: 1292
Cys Cys Phe Trp Cys Cys Asn Pro Ala Cys Thr
Gly Cys Tyr
(SEQ ID NO: 1293
Cys Cys Trp Trp Cys Cys Asn Pro Ala Cys Thr
Gly Cys Tyr
(SEQ ID NO: 1294
Cys Cys Gly Trp Cys Cys Asn Pro Ala Cys Thr
Gly Cys Tyr
(SEQ ID NO: 1295
Cys Cys Ser Trp Cys Cys Asn Pro Ala Cys Thr
Gly Cys Tyr
(SEQ ID NO: 1296
Cys Cys Thr Trp Cys Cys Asn Pro Ala Cys Thr
Gly Cys Tyr
(SEQ ID NO: 1297
Cys Cys Cys Trp Cys Cys Asn Pro Ala Cys Thr
Gly Cys Tyr
(SEQ ID NO: 1298
Cys Cys Asn Trp Cys Cys Asn Pro Ala Cys Thr
Gly Cys Tyr
(SEQ ID NO: 1299
Cys Cys Gln Trp Cys Cys Asn Pro Ala Cys Thr
Gly Cys Tyr
(SEQ ID NO: 1300
Cys Cys Tyr Trp Cys Cys Asn Pro Ala Cys Thr
Gly Cys Tyr
(SEQ ID NO: 1301
Cys Cys Asp Trp Cys Cys Asn Pro Ala Cys Thr
Gly Cys Tyr
(SEQ ID NO: 1302
Cys Cys Lys Trp Cys Cys Asn Pro Ala Cys Thr
Gly Cys Tyr
(SEQ ID NO: 1303
Cys Cys Arg Trp Cys Cys Asn Pro Ala Cys Thr
Gly Cys Tyr
(SEQ ID NO: 1304
Cys Cys His Trp Cys Cys Asn Pro Ala Cys Thr
Gly Cys Tyr
(SEQ ID NO: 1305
Cys Cys Ala Trp Cys Cys Asn Pro Ala Cys Thr
Gly Cys
(SEQ ID NO: 1306
Cys Cys Val Trp Cys Cys Asn Pro Ala Cys Thr
Gly Cys
(SEQ ID NO: 1307
Cys Cys Leu Trp Cys Cys Asn Pro Ala Cys Thr
Gly Cys
(SEQ ID NO: 1308
Cys Cys Ile Trp Cys Cys Asn Pro Ala Cys Thr
Gly Cys
(SEQ ID NO: 1309
Cys Cys Met Trp Cys Cys Asn Pro Ala Cys Thr
Gly Cys
(SEQ ID NO: 1310
Cys Cys Phe Trp Cys Cys Asn Pro Ala Cys Thr
Gly Cys
(SEQ ID NO: 1311
Cys Cys Trp Trp Cys Cys Asn Pro Ala Cys Thr
Gly Cys
(SEQ ID NO: 1312
Cys Cys Gly Trp Cys Cys Asn Pro Ala Cys Thr
Gly Cys
(SEQ ID NO: 1313
Cys Cys Ser Trp Cys Cys Asn Pro Ala Cys Thr
Gly Cys
(SEQ ID NO: 1314
Cys Cys Thr Trp Cys Cys Asn Pro Ala Cys Thr
Gly Cys
(SEQ ID NO: 1315
Cys Cys Cys Trp Cys Cys Asn Pro Ala Cys Thr
Gly Cys
(SEQ ID NO: 1316
Cys Cys Asn Trp Cys Cys Asn Pro Ala Cys Thr
Gly Cys
(SEQ ID NO: 1317
Cys Cys Gln Trp Cys Cys Asn Pro Ala Cys Thr
Gly Cys
(SEQ ID NO: 1318
Cys Cys Tyr Trp Cys Cys Asn Pro Ala Cys Thr
Gly Cys
(SEQ ID NO: 1319
Cys Cys Asp Trp Cys Cys Asn Pro Ala Cys Thr
Gly Cys
(SEQ ID NO: 1320
Cys Cys Lys Trp Cys Cys Asn Pro Ala Cys Thr
Gly Cys
(SEQ ID NO: 1321
Cys Cys Arg Trp Cys Cys Asn Pro Ala Cys Thr
Gly Cys;
and
(SEQ ID NO: 1322
Cys Cys His Trp Cys Cys Asn Pro Ala Cys Thr
Gly Cys

Additional useful peptides include peptides wherein D′ comprises, consists of, or consists essentially of the amino acid sequence (SEQ ID NOs:1423-1550, respectively, as shown below):

(SEQ ID NO: 1423
Cys Glu Leu Cys Ile Asn Val Ala Cys Thr Gly Cys
(SEQ ID NO: 1424
Cys Glu Leu Cys Val Asn Val Ala Cys Thr Gly Cys
(SEQ ID NO: 1425
Cys Ala Glu Leu Cys Cys Asn Pro Ala Cys
(SEQ ID NO: 1426
Cys Cys Gly Leu Cys Cys Asn Pro Ala Cys Ala Gly Cys
(SEQ ID NO: 1427
Cys Cys Gly Leu Cys Cys Tyr Pro Ala Cys Ala Gly Cys
(SEQ ID NO: 1428
Cys Glu Leu Cys Cys Asn Pro Ala Cys Ala Gly Cys
(SEQ ID NO: 1429
Cys Cys Asp Val Cys Cys Tyr Pro Ala Cys Thr Gly Cys
(SEQ ID NO: 1430
Cys Cys Glu Leu Cys Cys Asn Pro Ala Cys Ala Gly Cys
(SEQ ID NO: 1431
Cys Cys Glu Leu Cys Cys Tyr Pro Ala Cys Ala Gly Cys
(SEQ ID NO: 1432
Cys Cys Glu Leu Cys Cys Asn Pro Ala Cys Thr Gly Cys
(SEQ ID NO: 1433
Cys Cys Glu Leu Cys Cys Tyr Pro Ala Cys Thr Gly Cys
(SEQ ID NO: 1434
Cys Cys Glu Leu Cys Cys Asn Pro Gly Cys Thr Gly Cys
(SEQ ID NO: 1435
Cys Cys Glu Ala Cys Cys Asn Pro Ala Cys Thr Gly Cys
(SEQ ID NO: 1436
Cys Cys Glu Lys Cys Cys Asn Pro Ala Cys Thr Gly Cys
(SEQ ID NO: 1437
Cys Cys Glu Leu Cys Cys Asn Pro Ala Cys Thr Ala Cys
(SEQ ID NO: 1438
Cys Cys Pro Leu Cys Cys Asn Pro Ala Cys Thr Gly Cys
(SEQ ID NO: 1439
Ala Cys Glu Leu Cys Ala Asn Pro Ala Cys Thr Gly Cys
(SEQ ID NO: 1440
Cys Cys Glu Leu Ala Cys Asn Pro Ala Cys Thr Gly Ala
(SEQ ID NO: 1441
Cys Glu Leu Cys Ala Asn Pro Ala Cys Thr Gly Cys
(SEQ ID NO: 1442
Cys Cys Glu Leu Ala Cys Asn Pro Ala Cys
(SEQ ID NO: 1443
Cys Cys Asp Val Cys Cys Asn Pro Ala Cys Ala Gly Cys
(SEQ ID NO: 1444
Cys Cys Asp Val Cys Cys Asn Pro Ala Cys Thr Gly Cys
(SEQ ID NO: 1445
Cys Cys Asp Val Cys Cys Asn Pro Ala Cys Ala Gly Cys
Tyr
(SEQ ID NO: 1446
Cys Cys Asp Val Cys Cys Asn Pro Ala Cys Thr Gly Cys
Tyr
(SEQ ID NO: 1447
Cys Cys Glu Leu Cys Cys Tyr Pro Ala Cys Ala Gly Cys
(SEQ ID NO: 1448
Cys Cys Ile Cys Cys Asn Pro Ala Cys Phe Gly Cys
(SEQ ID NO: 1449
Cys Cys Asn Tyr Cys Cys Ser Pro Cys Gly Cys
(SEQ ID NO. 1450)
Cys Cys Asp Val Cys Cys Asn Pro Ala Cys Ala Gly Cys
(SEQ ID NO. 1451)
Cys Cys Asp Ala Cys Cys Asn Pro Ala Cys Ala Gly Cys
(SEQ ID NO. 1452)
Cys Cys Asp Cys Cys Cys Asn Pro Ala Cys Ala Gly Cys
(SEQ ID NO. 1453)
Cys Cys Asp Asp Cys Cys Asn Pro Ala Cys Ala Gly Cys
(SEQ ID NO. 1454)
Cys Cys Asp Glu Cys Cys Asn Pro Ala Cys Ala Gly Cys
(SEQ ID NO. 1455)
Cys Cys Asp Phe Cys Cys Asn Pro Ala Cys Ala Gly Cys
(SEQ ID NO. 1456)
Cys Cys Asp Gly Cys Cys Asn Pro Ala Cys Ala Gly Cys
(SEQ ID NO. 1457)
Cys Cys Asp His Cys Cys Asn Pro Ala Cys Ala Gly Cys
(SEQ ID NO. 1458)
Cys Cys Asp Ile Cys Cys Asn Pro Ala Cys Ala Gly Cys
(SEQ ID NO. 1459)
Cys Cys Asp Lys Cys Cys Asn Pro Ala Cys Ala Gly Cys
(SEQ ID NO. 1460)
Cys Cys Asp Leu Cys Cys Asn Pro Ala Cys Ala Gly Cys
(SEQ ID NO. 1461)
Cys Cys Asp Met Cys Cys Asn Pro Ala Cys Ala Gly Cys
(SEQ ID NO. 1462)
Cys Cys Asp Asn Cys Cys Asn Pro Ala Cys Ala Gly Cys
(SEQ ID NO. 1463)
Cys Cys Asp Pro Cys Cys Asn Pro Ala Cys Ala Gly Cys
(SEQ ID NO. 1464)
Cys Cys Asp Gln Cys Cys Asn Pro Ala Cys Ala Gly Cys
(SEQ ID NO. 1465)
Cys Cys Asp Arg Cys Cys Asn Pro Ala Cys Ala Gly Cys
(SEQ ID NO. 1466)
Cys Cys Asp Ser Cys Cys Asn Pro Ala Cys Ala Gly Cys
(SEQ ID NO. 1467)
Cys Cys Asp Thr Cys Cys Asn Pro Ala Cys Ala Gly Cys
(SEQ ID NO. 1468)
Cys Cys Asp Trp Cys Cys Asn Pro Ala Cys Ala Gly Cys
(SEQ ID NO. 1469)
Cys Cys Asp Tyr Cys Cys Asn Pro Ala Cys Ala Gly Cys
(SEQ ID NO. 1470)
Cys Cys Glu Ile Cys Cys Asn Pro Ala Cys Phe Gly Cys
(SEQ ID NO. 1471)
Cys Cys Glu Ala Cys Cys Asn Pro Ala Cys Phe Gly Cys
(SEQ ID NO. 1472)
Cys Cys Glu Cys Cys Cys Asn Pro Ala Cys Phe Gly Cys
(SEQ ID NO. 1473)
Cys Cys Glu Asp Cys Cys Asn Pro Ala Cys Phe Gly Cys
(SEQ ID NO. 1474)
Cys Cys Glu Glu Cys Cys Asn Pro Ala Cys Phe Gly Cys
(SEQ ID NO. 1475)
Cys Cys Glu Phe Cys Cys Asn Pro Ala Cys Phe Gly Cys
(SEQ ID NO. 1476)
Cys Cys Glu Gly Cys Cys Asn Pro Ala Cys Phe Gly Cys
(SEQ ID NO. 1477)
Cys Cys Glu His Cys Cys Asn Pro Ala Cys Phe Gly Cys
(SEQ ID NO. 1478)
Cys Cys Glu Lys Cys Cys Asn Pro Ala Cys Phe Gly Cys
(SEQ ID NO. 1479)
Cys Cys Glu Leu Cys Cys Asn Pro Ala Cys Phe Gly Cys
(SEQ ID NO. 1480)
Cys Cys Glu Met Cys Cys Asn Pro Ala Cys Phe Gly Cys
(SEQ ID NO. 1481)
Cys Cys Glu Asn Cys Cys Asn Pro Ala Cys Phe Gly Cys
(SEQ ID NO. 1482)
Cys Cys Glu Pro Cys Cys Asn Pro Ala Cys Phe Gly Cys
(SEQ ID NO. 1483)
Cys Cys Glu Gln Cys Cys Asn Pro Ala Cys Phe Gly Cys
(SEQ ID NO. 1484)
Cys Cys Glu Arg Cys Cys Asn Pro Ala Cys Phe Gly Cys
(SEQ ID NO. 1485)
Cys Cys Glu Ser Cys Cys Asn Pro Ala Cys Phe Gly Cys
(SEQ ID NO. 1486)
Cys Cys Glu Thr Cys Cys Asn Pro Ala Cys Phe Gly Cys
(SEQ ID NO. 1487)
Cys Cys Glu Val Cys Cys Asn Pro Ala Cys Phe Gly Cys
(SEQ ID NO. 1488)
Cys Cys Glu Trp Cys Cys Asn Pro Ala Cys Phe Gly Cys
(SEQ ID NO. 1489)
Cys Cys Glu Tyr Cys Cys Asn Pro Ala Cys Phe Gly Cys
(SEQ ID NO. 1490)
Cys Cys Glu Leu Cys Cys Asn Pro Ala Cys Ala Pro Cys
(SEQ ID NO. 1491)
Cys Cys Glu Ala Cys Cys Asn Pro Ala Cys Ala Pro Cys
(SEQ ID NO. 1492)
Cys Cys Glu Cys Cys Cys Asn Pro Ala Cys Ala Pro Cys
(SEQ ID NO. 1493)
Cys Cys Glu Asp Cys Cys Asn Pro Ala Cys Ala Pro Cys
(SEQ ID NO. 1494)
Cys Cys Glu Glu Cys Cys Asn Pro Ala Cys Ala Pro Cys
(SEQ ID NO. 1495)
Cys Cys Glu Phe Cys Cys Asn Pro Ala Cys Ala Pro Cys
(SEQ ID NO. 1496)
Cys Cys Glu Gly Cys Cys Asn Pro Ala Cys Ala Pro Cys
(SEQ ID NO. 1497)
Cys Cys Glu His Cys Cys Asn Pro Ala Cys Ala Pro Cys
(SEQ ID NO. 1498)
Cys Cys Glu Ile Cys Cys Asn Pro Ala Cys Ala Pro Cys
(SEQ ID NO. 1499)
Cys Cys Glu Lys Cys Cys Asn Pro Ala Cys Ala Pro Cys
(SEQ ID NO. 1500)
Cys Cys Glu Met Cys Cys Asn Pro Ala Cys Ala Pro Cys
(SEQ ID NO. 1501)
Cys Cys Glu Asn Cys Cys Asn Pro Ala Cys Ala Pro Cys
(SEQ ID NO. 1502)
Cys Cys Glu Pro Cys Cys Asn Pro Ala Cys Ala Pro Cys
(SEQ ID NO. 1503)
Cys Cys Glu Gln Cys Cys Asn Pro Ala Cys Ala Pro Cys
(SEQ ID NO. 1504)
Cys Cys Glu Arg Cys Cys Asn Pro Ala Cys Ala Pro Cys
(SEQ ID NO. 1505)
Cys Cys Glu Ser Cys Cys Asn Pro Ala Cys Ala Pro Cys
(SEQ ID NO. 1506)
Cys Cys Glu Thr Cys Cys Asn Pro Ala Cys Ala Pro Cys
(SEQ ID NO. 1507)
Cys Cys Glu Val Cys Cys Asn Pro Ala Cys Ala Pro Cys
(SEQ ID NO. 1508)
Cys Cys Glu Trp Cys Cys Asn Pro Ala Cys Ala Pro Cys
(SEQ ID NO. 1509)
Cys Cys Glu Tyr Cys Cys Asn Pro Ala Cys Ala Pro Cys
(SEQ ID NO. 1510)
Cys Cys Glu Leu Cys Cys Asn Pro Ala Cys Phe Gly Cys
(SEQ ID NO. 1511)
Cys Cys Glu Ala Cys Cys Asn Pro Ala Cys Phe Gly Cys
(SEQ ID NO. 1512)
Cys Cys Glu Cys Cys Cys Asn Pro Ala Cys Phe Gly Cys
(SEQ ID NO. 1513)
Cys Cys Glu Asp Cys Cys Asn Pro Ala Cys Phe Gly Cys
(SEQ ID NO. 1514)
Cys Cys Glu Glu Cys Cys Asn Pro Ala Cys Phe Gly Cys
(SEQ ID NO. 1515)
Cys Cys Glu Phe Cys Cys Asn Pro Ala Cys Phe Gly Cys
(SEQ ID NO. 1516)
Cys Cys Glu Gly Cys Cys Asn Pro Ala Cys Phe Gly Cys
(SEQ ID NO. 1517)
Cys Cys Glu His Cys Cys Asn Pro Ala Cys Phe Gly Cys
(SEQ ID NO. 1518)
Cys Cys Glu Ile Cys Cys Asn Pro Ala Cys Phe Gly Cys
(SEQ ID NO. 1519)
Cys Cys Glu Lys Cys Cys Asn Pro Ala Cys Phe Gly Cys
(SEQ ID NO. 1520)
Cys Cys Glu Met Cys Cys Asn Pro Ala Cys Phe Gly Cys
(SEQ ID NO. 1521)
Cys Cys Glu Asn Cys Cys Asn Pro Ala Cys Phe Gly Cys
(SEQ ID NO. 1522)
Cys Cys Glu Pro Cys Cys Asn Pro Ala Cys Phe Gly Cys
(SEQ ID NO. 1523)
Cys Cys Glu Gln Cys Cys Asn Pro Ala Cys Phe Gly Cys
(SEQ ID NO. 1524)
Cys Cys Glu Arg Cys Cys Asn Pro Ala Cys Phe Gly Cys
(SEQ ID NO. 1525)
Cys Cys Glu Ser Cys Cys Asn Pro Ala Cys Phe Gly Cys
(SEQ ID NO. 1526)
Cys Cys Glu Thr Cys Cys Asn Pro Ala Cys Phe Gly Cys
(SEQ ID NO. 1527)
Cys Cys Glu Val Cys Cys Asn Pro Ala Cys Phe Gly Cys
(SEQ ID NO. 1528)
Cys Cys Glu Trp Cys Cys Asn Pro Ala Cys Phe Gly Cys
(SEQ ID NO. 1529)
Cys Cys Glu Tyr Cys Cys Asn Pro Ala Cys Phe Gly Cys
(SEQ ID NO. 1530)
Cys Cys Glu Val Cys Cys Asn Pro Ala Cys Ala Gly Cys
(SEQ ID NO. 1531)
Cys Cys Glu Ala Cys Cys Asn Pro Ala Cys Ala Gly Cys
(SEQ ID NO. 1532)
Cys Cys Glu Cys Cys Cys Asn Pro Ala Cys Ala Gly Cys
(SEQ ID NO. 1533)
Cys Cys Glu Asp Cys Cys Asn Pro Ala Cys Ala Gly Cys
(SEQ ID NO. 1534)
Cys Cys Glu Glu Cys Cys Asn Pro Ala Cys Ala Gly Cys
(SEQ ID NO. 1535)
Cys Cys Glu Phe Cys Cys Asn Pro Ala Cys Ala Gly Cys
(SEQ ID NO. 1536)
Cys Cys Glu Gly Cys Cys Asn Pro Ala Cys Ala Gly Cys
(SEQ ID NO. 1537)
Cys Cys Glu His Cys Cys Asn Pro Ala Cys Ala Gly Cys
(SEQ ID NO. 1538)
Cys Cys Glu Ile Cys Cys Asn Pro Ala Cys Ala Gly Cys
(SEQ ID NO. 1539)
Cys Cys Glu Lys Cys Cys Asn Pro Ala Cys Ala Gly Cys
(SEQ ID NO. 1540)
Cys Cys Glu Leu Cys Cys Asn Pro Ala Cys Ala Gly Cys
(SEQ ID NO. 1541)
Cys Cys Glu Met Cys Cys Asn Pro Ala Cys Ala Gly Cys
(SEQ ID NO. 1542)
Cys Cys Glu Asn Cys Cys Asn Pro Ala Cys Ala Gly Cys
(SEQ ID NO. 1543)
Cys Cys Glu Pro Cys Cys Asn Pro Ala Cys Ala Gly Cys
(SEQ ID NO. 1544)
Cys Cys Glu Gln Cys Cys Asn Pro Ala Cys Ala Gly Cys
(SEQ ID NO. 1545)
Cys Cys Glu Arg Cys Cys Asn Pro Ala Cys Ala Gly Cys
(SEQ ID NO. 1546)
Cys Cys Glu Ser Cys Cys Asn Pro Ala Cys Ala Gly Cys
(SEQ ID NO. 1547)
Cys Cys Glu Thr Cys Cys Asn Pro Ala Cys Ala Gly Cys
(SEQ ID NO. 1548)
Cys Cys Glu Trp Cys Cys Asn Pro Ala Cys Ala Gly Cys
(SEQ ID NO. 1549)
Cys Cys Glu Tyr Cys Cys Asn Pro Ala Cys Ala Gly Cys

Also useful are the following peptides (SEQ ID NOs: 1551-1553, respectively, as shown below) wherein Xaa represents any of the 20 naturally occurring amino acids

(SEQ ID NO: 1551)
Cys Cys Xaa Xaa Cys Cys Xaa Pro Ala Cys Xaa Gly Cys
(SEQ ID NO: 1552)
Cys Cys Ile Xaa Cys Cys Asn Pro Ala Cys Phe Gly Cys
(SEQ ID NO: 1553)
Cys Cys Asn Tyr Cys Cys Ser Pro Xaa Cys Xaa Gly Cys

The disclosure also features deletion variants of any of the peptides described herein in which one, two, three or four amino acids (or non-natural amino acids or natural or non-natural amino acid analogs), other than a Cys (or an amino acid substituted for Cys, e.g, an amino acid capable of forming a covalent bond to another amino acid), are deleted. Where two (or more) amino acids are deleted and the peptide comprises the sequence: Cysₐ Cys_{b} Xaa Xaa Cys_{c} Cys_{d} Xaa Xaa Xaa Cysₑ Xaa Xaa Cys_{f}, in some cases two or more deletions can be located between Cys_{b} and Cys_{c} and/or between Cys_{d} and Cysₑ and/or between Cysₑ and Cys_{f}. However, in other cases there is at most one deletion between each of Cys_{b} and Cys_{c} or between Cys_{d} and Cysₑ or between Cysₑ and Cys_{f}. Thus, the disclosure includes any of the peptides described herein comprising the sequence Cysₐ Cys_{b} Xaa Xaa Cys_{c} Cys_{d} Xaa Xaa Xaa Cysₑ Xaa Xaa Cys_{f} wherein: a) one amino acid between Cys_{b} and Cys_{c} is deleted; b) one amino acid between Cys_{d} and Cysₑ is deleted; c) one amino acid between Cysₑ and Cys_{f} is deleted; d) one amino acid between Cys_{b} and Cys_{c} is deleted and one amino acid between Cys_{d} and Cysₑ is deleted; e) one amino acid between Cys_{d} and Cysₑ is deleted and one amino acid between Cysₑ and Cys_{f} is deleted; f) one amino acid between Cys_{b} and Cys_{c} is deleted and one amino acid between Cysₑ and Cys_{f} is deleted or g) one amino acid between Cys_{b} and Cys_{c} is deleted, one amino acid between Cys_{d} and Cysₑ is deleted and one amino acid between Cysₑ and Cys_{f} is deleted. In certain cases, the various deletion variants are peptides that bind to and/or activate the GC-C receptor. In various cases, the various deletion variants are peptides that increase cGMP levels.

Deletion variants of Cys Cys Glu Tyr Cys Cys Asn Pro Ala Cys Thr Gly Cys Tyr (SEQ ID NO:805) include the peptides listed in FIG. 13. In these deletion variants, any of the amino acids can be deleted and there can be one, two, three or four amino acids deleted other than Cys.

The disclosure also features insertion variants of any of the peptides described herein in which one, two, three or four amino acids (e.g., Gly or Ala) are inserted before or after any amino acid in the peptide. In some cases no more than one amino acid is inserted between two Cys. For example, where two or more amino acids are inserted and the peptide comprises the sequence Cysₐ Cys_{b} Xaa Xaa Cys_{c} Cys_{d} Xaa Xaa Xaa Cysₑ Xaa Xaa Cys_{f} (SEQ ID NO:1554), in some cases two or more insertions can be located between Cys_{b} and Cys_{c} or between Cys_{d} and Cysₑ or between Cysₑ and Cys_{f}. However, in other cases no more than one insertion is located between Cys_{b} and Cys_{c} or between Cys_{d} and Cysₑ or between Cysₑ and Cys_{f}. Thus, the disclosure features any of the peptides described herein comprising the sequence Cysₐ Cys_{b} Xaa Xaa Cys_{c} Cys_{d} Xaa Xaa Xaa Cysₑ Xaa Xaa Cys_{f} (SEQ ID NO:1554) wherein: a) one amino acid is inserted between Cys_{b} and Cys_{c}; b) one amino acid is inserted between Cys_{d} and Cysₑ; c) one amino acid is inserted between Cysₑ and Cys_{f}; d) one amino acid is inserted between Cys_{b} and Cys_{c} and one amino acid is inserted between Cys_{d} and Cysₑ; e) one amino acid is inserted between Cys_{d} and Cysₑ and one amino acid is inserted between Cysₑ and Cys_{f}; f) one amino acid is inserted between Cys_{b} and Cys_{c} and one amino acid is inserted between Cysₑ and Cys_{f}; or g) one amino acid is inserted between Cys_{b} and Cys_{c}, one amino acid is inserted between Cys_{d} and Cysₑ and one amino acid is inserted between Cysₑ and Cys_{f}. In addition, one or more amino acids can be inserted preceding Cysₐ and/or one or more amino acids can be inserted following Cys_{f}.

Insertion variants of Cys Cys Glu Tyr Cys Cys Asn Pro Ala Cys Thr Gly Cys Tyr (SEQ ID NO:805) include those in which up to four amino acids (i.e., 0, 1, 2, 3 or 4) can be inserted after each amino acid. Thus, the disclosure includes peptides having the sequence: Cys Xaa₍₀₋₄₎ Cys Xaa₍₀₋₄₎ Glu Xaa₍₀₋₄₎ Tyr Xaa₍₀₋₄₎ Cys Xaa₍₀₋₄₎ Cys Xaa₍₀₋₄₎ Asn Xaa₍₀₋₄₎ Pro Xaa₍₀₋₄₎ Ala Xaa₍₀₋₄₎ Cys Xaa₍₀₋₄₎ Thr Xaa₍₀₋₄₎ Gly Xaa₍₀₋₄₎ Cys Xaa₍₀₋₄₎ Tyr Xaa₍₀₋₄₎₎ (SEQ ID NO: 1555). The inserted amino acids can be any amino acid or amino acid analog (natural or non-natural) and can be the same or different. In certain cases the inserted amino acids are all Gly or all Ala or a combination of Gly and Ala.

FIG. 14 depicts insertion variants of the peptide having the sequence: Cys Cys Glu Tyr Cys Cys Asn Pro Ala Cys Thr Gly Cys Tyr (SEQ ID NO:805).

The disclosure also features variants of peptides having the sequence Xaa₁ Xaa₂ Xaa₃ Xaa₄ Xaa₅ Cys₆ Cys₇ Xaa₈ Xaa₉ Cys₁₀ Cys₁₁ Xaa₁₂ Xaa₁₃ Xaa₁₄ Cys₁₅ Xaa₁₆ Xaa₁₇ Cys₁₈ Xaa₁₉ Xaa₂₀ Xaa₂₁ (SEQ ID NO:63), e.g., variants of Cys Cys Glu Tyr Cys Cys Asn Pro Ala Cys Thr Gly Cys Tyr (SEQ ID NO:805), in which up to four amino acids are deleted and/or up to four amino acids are inserted. The insertions and deletions can be between Cys₆ and Cys₁₈ in SEQ ID NO:63 or they can be amino terminal to Cys₆ and/or carboxy terminal to Cys₁₈ in SEQ ID NO:63.

In some cases, the polypeptides described herein include polypeptides comprising (consisting essentially of or consisting of) the amino acid sequence:

(SEQ ID NO: 1556)
X₁ Cys Glu X₂ X₃ X₄ Asn Pro Ala Cys Thr Gly X₅ X₆

wherein:
X₁, X₃, X₄ and X₅ are independently selected from: Ala, Arg, Asn, Asp, Cys, Gln, Glu, Gly, His, Ile, Leu, Lys, Met, Phe, Pro, Ser, Thr, Trp, Tyr and Val;
X₂ is selected from: Ala, Arg, Asn, Asp, Cys, Gln, Glu, Gly, His, Ile, Leu, Lys, Met, Phe, Pro, Ser, Thr, Trp, Tyr and Val; and
X₆ is selected from Phe, Trp and Tyr or is missing,

provided that when both X₁ and X₄ are Ala and both X₃ and X₅ are Cys or when both X₃ and X₅ are Ala and both X₁ and X₄ are Cys or when X₁, X₃, X₄ and X₅ are all Cys, then either X₆ is selected from Phe and Trp or X₂ is not Leu.

In various cases of X₁ Cys Glu X₂ X₃ X₄ Asn Pro Ala Cys Thr Gly X₅ X₆ (SEQ ID NO:1556): at least one of X₁, X₃, X₄ and X₅ is Cys; at least two of X₁, X₃, X₄ and X₅ are Cys; at least three of X₁, X₃, X₄ and X₅ is Cys; X₁, X₃, X₄ and X₅ are Cys; X₁ and X₄ are Cys; X₃ and X₅ are Gly or Ala; X₃ and X₅ are Cys; X₁ and X₄ are Gly or Ala; X₁, X₃, X₄ and X₅ are Cys; X₂ is selected from: Ala, Arg, Asn, Asp, Cys, Gln, Glu, Gly, His, Ile, Lys, Met, Phe, Pro, Ser, Thr, Val, Trp and Tyr; one of X₁, X₃, X₄ and X₅ is Gly or Ala and the rest are Cys; two of X₁, X₃, X₄ and X₅ are Gly or Ala and the rest are Cys; three of X₁, X₃, X₄ and X₅ are Gly or Ala and the rest are Cys; X₁ and X₄ are independently Gly or Ala and X₃ and X₅ are Cys; X₃ and X₅ are independent Gly or Ala and X₁ and X₄ are Cys; X₂ is Phe, Tyr or Trp; X₂ is Phe; X₂ is Tyr; X₂ is Trp; X₆ is Tyr; X₆ is missing; X₁ is Gly or Ala; X₃ is Gly or Ala; X₄ is Gly or Ala; X₅ is Gly or Ala; X₁ and X₄ are Ala and X₃ and X₅ are Cys; X₃ and X₅ are Ala and X₁ and X₄ are Cys; X₁ and X₄ are Gly and X₃ and X₅ are Cys; X₃ and X₅ are Gly and X₁ and X₄ are Cys; one of X₁ and X₄ is Ala and the other is Gly and X₃ and X₅ are Cys; an one X₃ and X₅ is Ala and the other is Gly and X₁ and X₄ are Cys; the polypeptide comprises 100 or fewer amino acids; the polypeptide comprises 20 or fewer amino acids; the polypeptide comprises 15 or fewer amino acids. Additional cases are shown in FIG. 10.

The variants of the forgoing polypeptides can be created by insertion or deletion of amino acids. For example, one or two amino acids within the sequence X₁ Cys Glu X₂ X₃ X₄ Asn Pro Ala Cys Thr Gly X₅ X₆ (SEQ ID NO:1556) can be deleted. The deleted amino acids can be selected from Glu, X₂, Asn, Pro, Ala, Thr and Gly in the sequence X₁ Cys Glu X₂ X₃ X₄ Asn Pro Ala Cys Thr Gly X₅ X₆. In addition, insertions of 1, 2, 3, or 4 contiguous amino acids into a peptide having the sequence X₁ Cys Glu X₂ X₃ X₄ Asn Pro Ala Cys Thr Gly X₅ X₆ (SEQ ID NO:1556) can be made. Preferably the insertions are not between X₁ and Cys or between X₅ X₆ in a peptide having the sequence X₁ Cys Glu X₂ X₃ X₄ Asn Pro Ala Cys Thr Gly X₅ X₆ (SEQ ID NO:1556). Various insertion and deletion variants are depicted in FIGS. 11 and 12 (Xaa represents any amino acid.

In some cases, GC-C receptor binding polypeptides (D′) that can modified by the addition of pre, pro, prepro, N-terminal non-core, and C-terminal non-core sequences are SEQ ID NOs:1557-1594, 805, and 1595, respectively, as shown below:

(SEQ ID NO: 1557)
Cys Cys Glu Ala Cys Cys Asn Pro Ala Cys Thr Gly Cys
(SEQ ID NO: 1558)
Cys Cys Glu Arg Cys Cys Asn Pro Ala Cys Thr Gly Cys
(SEQ ID NO: 1559)
Cys Cys Glu Asn Cys Cys Asn Pro Ala Cys Thr Gly Cys
(SEQ ID NO: 1560)
Cys Cys Glu Asp Cys Cys Asn Pro Ala Cys Thr Gly Cys
(SEQ ID NO: 1561)
Cys Cys Glu Cys Cys Cys Asn Pro Ala Cys Thr Gly Cys
(SEQ ID NO: 1562)
Cys Cys Glu Gln Cys Cys Asn Pro Ala Cys Thr Gly Cys
(SEQ ID NO: 1563)
Cys Cys Glu Glu Cys Cys Asn Pro Ala Cys Thr Gly Cys
(SEQ ID NO: 1564)
Cys Cys Glu Gly Cys Cys Asn Pro Ala Cys Thr Gly Cys
(SEQ ID NO: 1565)
Cys Cys Glu His Cys Cys Asn Pro Ala Cys Thr Gly Cys
(SEQ ID NO: 1566)
Cys Cys Glu Ile Cys Cys Asn Pro Ala Cys Thr Gly Cys
(SEQ ID NO: 1567)
Cys Cys Glu Lys Cys Cys Asn Pro Ala Cys Thr Gly Cys
(SEQ ID NO: 1568)
Cys Cys Glu Met Cys Cys Asn Pro Ala Cys Thr Gly Cys
(SEQ ID NO: 1569)
Cys Cys Glu Phe Cys Cys Asn Pro Ala Cys Thr Gly Cys
(SEQ ID NO: 1570)
Cys Cys Glu Pro Cys Cys Asn Pro Ala Cys Thr Gly Cys
(SEQ ID NO: 1571)
Cys Cys Glu Ser Cys Cys Asn Pro Ala Cys Thr Gly Cys
(SEQ ID NO: 1572)
Cys Cys Glu Thr Cys Cys Asn Pro Ala Cys Thr Gly Cys
(SEQ ID NO: 1573)
Cys Cys Glu Trp Cys Cys Asn Pro Ala Cys Thr Gly Cys
(SEQ ID NO: 1574)
Cys Cys Glu Val Cys Cys Asn Pro Ala Cys Thr Gly Cys
(SEQ ID NO: 1575)
Cys Cys Glu Tyr Cys Cys Asn Pro Ala Cys Thr Gly Cys
(SEQ ID NO: 1576)
Cys Cys Glu Leu Cys Cys Asn Pro Ala Cys Thr Gly Cys
(SEQ ID NO: 1577)
Cys Cys Glu Ala Cys Cys Asn Pro Ala Cys Thr Gly Cys
Tyr
(SEQ ID NO: 1578)
Cys Cys Glu Arg Cys Cys Asn Pro Ala Cys Thr Gly Cys
Tyr
(SEQ ID NO: 1579)
Cys Cys Glu Asn Cys Cys Asn Pro Ala Cys Thr Gly Cys
Tyr
(SEQ ID NO: 1580)
Cys Cys Glu Asp Cys Cys Asn Pro Ala Cys Thr Gly Cys
Tyr
(SEQ ID NO: 1581)
Cys Cys Glu Cys Cys Cys Asn Pro Ala Cys Thr Gly Cys
Tyr
(SEQ ID NO: 1582)
Cys Cys Glu Gln Cys Cys Asn Pro Ala Cys Thr Gly Cys
Tyr
(SEQ ID NO: 1583)
Cys Cys Glu Glu Cys Cys Asn Pro Ala Cys Thr Gly Cys
Tyr
(SEQ ID NO: 1584)
Cys Cys Glu Gly Cys Cys Asn Pro Ala Cys Thr Gly Cys
Tyr
(SEQ ID NO: 1585)
Cys Cys Glu His Cys Cys Asn Pro Ala Cys Thr Gly Cys
Tyr
(SEQ ID NO: 1586)
Cys Cys Glu Ile Cys Cys Asn Pro Ala Cys Thr Gly Cys
Tyr
(SEQ ID NO: 1587)
Cys Cys Glu Lys Cys Cys Asn Pro Ala Cys Thr Gly Cys
Tyr
(SEQ ID NO: 1588)
Cys Cys Glu Met Cys Cys Asn Pro Ala Cys Thr Gly Cys
Tyr
(SEQ ID NO: 1589)
Cys Cys Glu Phe Cys Cys Asn Pro Ala Cys Thr Gly Cys
Tyr
(SEQ ID NO: 1590)
Cys Cys Glu Pro Cys Cys Asn Pro Ala Cys Thr Gly Cys
Tyr
(SEQ ID NO: 1591)
Cys Cys Glu Ser Cys Cys Asn Pro Ala Cys Thr Gly Cys
Tyr
(SEQ ID NO: 1592)
Cys Cys Glu Thr Cys Cys Asn Pro Ala Cys Thr Gly Cys
Tyr
(SEQ ID NO: 1593)
Cys Cys Glu Trp Cys Cys Asn Pro Ala Cys Thr Gly Cys
Tyr
(SEQ ID NO: 1594)
Cys Cys Glu Val Cys Cys Asn Pro Ala Cys Thr Gly Cys
Tyr
(SEQ ID NO: 805)
Cys Cys Glu Tyr Cys Cys Asn Pro Ala Cys Thr Gly Cys
Tyr
(SEQ ID NO: 1596)
Cys Cys Glu Leu Cys Cys Asn Pro Ala Cys Thr Gly Cys
Tyr

In some cases, D′ can be based on a sequence comprising, consisting of, or consisting essentially of the sequence: Cys Cys Glu Leu Cys Cys Asn Pro Ala Cys Thr Gly Cys Tyr (SEQ ID NO:1596). To create a variant having a potentially functional chymotrypsin cleavage site capable of inactivating the peptide, either the Leu (underlined) or the Thr (underlined) can be replaced by Trp, Phe or Tyr or both the Leu and the Thr can be replaced by (independently) Trp, Phe or Tyr. To create a variant having an analgesic di-peptide, the core sequence is followed by Asp Phe. The carboxy terminal Tyr in the core sequence can allow the Asp Phe dipeptide to be released by chymotrypsin in the digestive tract.

Useful variants of SEQ ID NO:1596 include, but are not limited to SEQ ID NOs: 1597-1601, 805, and 1602-1629:

(SEQ ID NO: 1597)
Asn Ser Ser Asn Tyr Cys Cys Glu Leu Cys Cys Asn Pro
Ala Cys Thr Gly Cys Tyr
(SEQ ID NO: 1598)
Asn Ser Ser Asn Tyr Cys Cys Glu Leu Cys Cys Asn Pro
Ala Cys Trp Gly Cys Tyr
(SEQ ID NO: 1550)
Asn Ser Ser Asn Tyr Cys Cys Glu Tyr Cys Cys Asn Pro
Ala Cys Thr Gly Cys Tyr
NSSNYCCEYCCNPACTGCY
(SEQ ID NO: 1599)
Cys Cys Glu Leu Cys Cys Asn Pro Ala Cys Thr Gly Cys
Tyr
(SEQ ID NO: 1600)
Cys Cys Glu Leu Cys Cys Asn Pro Ala Cys Trp Gly Cys
Tyr
(SEQ ID NO: 805)
Cys Cys Glu Tyr Cys Cys Asn Pro Ala Cys Thr Gly Cys
Tyr
(SEQ ID NO: 1601)
Asn Cys Cys Glu Leu Cys Cys Asn Pro Ala Cys Thr Gly
Cys Tyr
(SEQ ID NO: 1602)
Asn Cys Cys Glu Leu Cys Cys Asn Pro Ala Cys Trp Gly
Cys Tyr
(SEQ ID NO: 1603)
Asn Cys Cys Glu Phe Cys Cys Asn Pro Ala Cys Thr Gly
Cys Tyr
(SEQ ID NO: 1604)
Asn Cys Cys Glu Tyr Cys Cys Asn Pro Ala Cys Thr Gly
Cys Tyr
(SEQ ID NO: 1605)
Asn Cys Cys Glu Trp Cys Cys Asn Pro Ala Cys Thr Gly
Cys Tyr
(SEQ ID NO: 1606)
Asn Cys Cys Glu Arg Cys Cys Asn Pro Ala Cys Thr Gly
Cys Tyr
(SEQ ID NO: 1607)
Asn Cys Cys Glu Lys Cys Cys Asn Pro Ala Cys Thr Gly
Cys Tyr
(SEQ ID NO: 1608)
Asn Ser Ser Asn Tyr Cys Cys Glu Leu Cys Cys Asn Pro
Ala Cys Thr Gly Cys Tyr Asp Phe
(SEQ ID NO: 1609)
Asn Ser Ser Asn Tyr Cys Cys Glu Leu Cys Cys Asn Pro
Ala Cys Trp Gly Cys Tyr Asp Phe
(SEQ ID NO: 1610)
Asn Ser Ser Asn Tyr Cys Cys Glu Phe Cys Cys Asn Pro
Ala Cys Thr Gly Cys Tyr Asp Phe
(SEQ ID NO: 1611)
Asn Ser Ser Asn Tyr Cys Cys Glu Tyr Cys Cys Asn Pro
Ala Cys Thr Gly Cys Tyr Asp Phe
(SEQ ID NO: 1612)
Asn Ser Ser Asn Tyr Cys Cys Glu Trp Cys Cys Asn Pro
Ala Cys Thr Gly Cys Tyr Asp Phe
(SEQ ID NO: 1613)
Asn Ser Ser Asn Tyr Cys Cys Glu Arg Cys Cys Asn Pro
Ala Cys Thr Gly Cys Tyr Asp Phe
(SEQ ID NO: 1614)
Asn Ser Ser Asn Tyr Cys Cys Glu Lys Cys Cys Asn Pro
Ala Cys Thr Gly Cys Tyr Asp Phe
(SEQ ID NO: 1615)
Cys Cys Glu Leu Cys Cys Asn Pro Ala Cys Thr Gly Cys
Tyr Asp Phe
(SEQ ID NO: 1616)
Cys Cys Glu Leu Cys Cys Asn Pro Ala Cys Trp Gly Cys
Tyr Asp Phe
(SEQ ID NO: 1617)
Cys Cys Glu Phe Cys Cys Asn Pro Ala Cys Thr Gly Cys
Tyr Asp Phe
(SEQ ID NO: 1618)
Cys Cys Glu Tyr Cys Cys Asn Pro Ala Cys Thr Gly Cys
Tyr Asp Phe
(SEQ ID NO: 1619)
Cys Cys Glu Trp Cys Cys Asn Pro Ala Cys Thr Gly Cys
Tyr Asp Phe
(SEQ ID NO: 1620)
Cys Cys Glu Arg Cys Cys Asn Pro Ala Cys Thr Gly Cys
Tyr Asp Phe
(SEQ ID NO: 1621)
Cys Cys Glu Lys Cys Cys Asn Pro Ala Cys Thr Gly Cys
Tyr Asp Phe
(SEQ ID NO: 1622)
Asn Cys Cys Glu Leu Cys Cys Asn Pro Ala Cys Thr Gly
Cys Tyr Asp Phe
(SEQ ID NO: 1623)
Asn Cys Cys Glu Leu Cys Cys Asn Pro Ala Cys Trp Gly
Cys Tyr Asp Phe
(SEQ ID NO: 1624)
Asn Cys Cys Glu Phe Cys Cys Asn Pro Ala Cys Thr Gly
Cys Tyr Asp Phe
(SEQ ID NO: 1625)
Asn Cys Cys Glu Tyr Cys Cys Asn Pro Ala Cys Thr Gly
Cys Tyr Asp Phe
(SEQ ID NO: 1626)
Asn Cys Cys Glu Trp Cys Cys Asn Pro Ala Cys Thr Gly
Cys Tyr Asp Phe
(SEQ ID NO: 1627)
Asn Cys Cys Glu Arg Cys Cys Asn Pro Ala Cys Thr Gly
Cys Tyr Asp Phe
(SEQ ID NO: 1628)
Asn Cys Cys Glu Lys Cys Cys Asn Pro Ala Cys Thr Gly
Cys Tyr Asp Phe

GC-C agonists of the disclosure can also comprise, consist essentially of, or consist of peptides derived from the C-terminal domain of any of the peptides described herein. Thus, they can contain, for example, anywhere from 13-75 amino acids including 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and/or 75 amino acids of the C-terminal domain of any of the peptides described herein.

In some cases it may be desirable to have a polypeptide that includes a pre sequence (A′) from a first bacterial ST polypeptide and a pro sequence (B′) from a second bacterial ST polypeptide. In other cases, the pre sequence (A′) and the pro sequence (B′) are from the same ST polypeptide.

### Cleavage of Polypeptides

While polypeptides can be cleaved at the carboxy terminal side of methionine using cyanogen bromide, other chemical and enzymatic cleavage methods can be used, for example to remove a pre-sequence and/or a pro-sequence. In selecting an appropriate cleavage method, it is important to make certain that cleavage sites are not located within the desired final polypeptide, i.e., within the GC-C agonist polypeptide.

Described below are various chemical and enzymatic cleavage methods and the sequence requirements for each. In the two tables below, P1 is the amino acid that is at amino terminal side of the cleavage site, and P1′ is the amino acid that is at the carboxy terminal side of the cleavage site. P2′ is immediately carboxy-terminal to P1′. P2 is immediately amino terminal to P1; P3 is immediately amino terminal to P2; and P4 is immediately amino terminal to P3. A “-” indicates that any amino acid can be present at that position. In some cases it is important that a particular amino acid is not present at a particular position relative to the cleavage site, and this is indicated in the tables.

In some cases it may be desirable to have two different cleavages. In such cases two different enzymes or chemicals (or an enzyme and a chemical) can be used. If the reactions conditions for the two cleavages are compatible and if it is desirable to do so, the two cleavages can take place in the same reaction. Alternatively, two separate reactions can be performed. In some cases purification can take place between the two different cleavages such that one or more polypeptide fragments are removed between the first and second cleavages. In other cases no such purification takes place.

Chemical cleavage

| chemical | P1 | P1′ |
|---|---|---|
| CNBr | M | — |
| Formic acid | D | — |
| Hydroxylamine | N | G |
| Iodosobenzoic | W | — |
| acid | | |

Enzymatic Cleavage

| Protease | P4 | P3 | P2 | P1 | P1′ | P2′ |
|---|---|---|---|---|---|---|
| Arg-C proteinase | — | — | — | R | — | — |
| Asp-N endopeptidase | — | — | — | — | D | — |
| BNPS-Skatole | — | — | — | W | — | — |
| Caspase 1 | F, W, Y, or L | — | H, A or T | D | not P, E, D, Q, | — |
| | | | | | K or R | |
| Caspase 2 | D | V | A | D | not P, E, D, Q, | — |
| | | | | | K or R | |
| Caspase 3 | D | M | Q | D | not P, E, D, Q, | — |
| | | | | | K or R | |
| Caspase 4 | L | E | V | D | not P, E, D, Q, | — |
| | | | | | K or R | |
| Caspase 5 | L or W | E | H | D | — | — |
| Caspase 6 | V | E | H or I | D | not P, E, D, Q, | — |
| | | | | | K or R | |
| Caspase 7 | D | E | V | D | not P, E, D, Q, | — |
| | | | | | K or R | |
| Caspase 8 | I or L | E | T | D | not P, E, D, Q, | — |
| | | | | | K or R | |
| Caspase 9 | L | E | H | D | — | — |
| Caspase 10 | I | E | A | D | — | — |
| Chymotrypsin-high | — | — | — | F or Y | not P | — |
| specificity | | | | | | |
| Chymotrypsin-high | — | — | — | W | not M or P | — |
| specificity | | | | | | |
| Chymotrypsin-low | — | — | — | F, L or Y | not P | — |
| specificity | | | | | | |
| Chymotrypsin-low | — | — | — | W | not M or P | — |
| specificity | | | | | | |
| Chymotrypsin-low | — | — | — | M | not P or Y | — |
| specificity | | | | | | |
| Chymotrypsin-low | — | — | — | H | not D, M, P | — |
| specificity | | | | | or W | |
| Clostripain | — | — | — | R | — | — |
| (Clostridiopeptidase B) | | | | | | |
| Enterokinase | D or N | D or N | D or N | K | — | — |
| Factor Xa | A, F, G, I, L, | D or E | G | R | — | — |
| | T, V or M | | | | | |
| Glutamyl | — | — | — | E | — | — |
| endopeptidase | | | | | | |
| GranzymeB | I | E | P | D | — | — |
| LysC | — | — | — | K | — | — |
| Pepsin (pH 1.3) | — | not H, K, or R | not P | not R | F, L, W or Y | not P |
| Pepsin (pH 1.3) | — | not H, K, or R | not P | F, L, W or Y | — | not P |
| Pepsin (pH > 2) | — | not H, K or R | not P | not R | F or L | not P |
| Pepsin (pH > 2) | — | not H, K or R | not P | F or L | — | not P |
| Proline-endopeptidase | — | — | H, K or R | P | not P | — |
| Proteinase K | — | — | — | A, E, F, I, L, | — | — |
| | | | | T, V, W or Y | | |
| Staphylococcal | — | — | not E | E | — | — |
| peptidase I | | | | | | |
| Thermolysin | — | — | — | not D or E | A, F, I, L, | — |
| | | | | | M or V | |
| Thrombin | — | — | G | R | G | — |
| Thrombin | A, F, G, I, L, | A, F, G, I, L, | P | R | not D or E | not DE |
| | T, V or M | T, V, W or A | | | | |
| Trypsin | — | — | — | K or R | not P | — |
| Trypsin | — | — | W | K | P | — |
| Trypsin | — | — | M | R | P | — |

### Asparagine Substitutions

In some cases, any of the polypeptides described herein may comprise Asn having the structure:

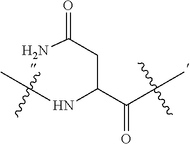

optionally replaced by a group having a structure selected from (a), (b) and (c):

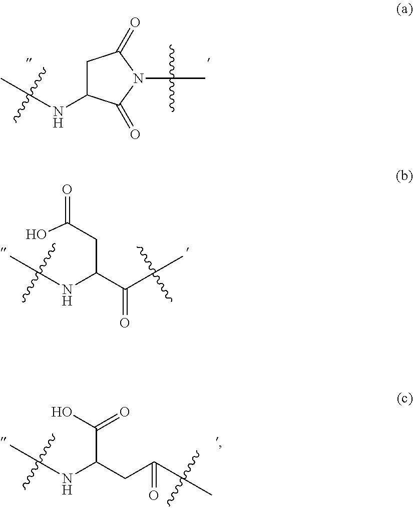

provided that an Asn at the carboxy terminus is not replaced by structure (a) or structure (c).

In various cases: at least one Asn is replaced by a group having structure (a at least one Asn is replaced by a group having structure (b); at least one Asn is replaced by a group having structure (c); an Asn at the amino terminus of the polypeptide is replaced by a structure selected from (a), (b) and (c); an Asn at the carboxy terminus of the polypeptide is replaced by a structure (b); an Asn that is neither at the carboxy terminus of the polypeptide nor the at the amino terminus of the polypeptide is replaced by a structure selected from (a), (b) and (c); all Asn are replaced by a structure selected from (a), (b) and (c); at least two Asn are replaced by a structure selected from (a), (b) and (c); at least three Asn are replaced by a structure selected from (a), (b) and (c); at least four Asn are replaced by a structure selected from (a), (b) and (c); at least five Asn are replaced by a structure selected from (a), (b) and (c); at least six Asn are replaced by a structure selected from (a), (b) and (c); all Asn replaced by a structure selected from (a), (b) and (c) are replaced by structure (a); all Asn replaced by a structure selected from (a), (b) and (c) are replaced by structure (b); all Asn replaced by a structure selected from (a), (b) and (c) are replaced by structure (c); at least one Asn within A′, when A′ is present, is replaced by a structure selected from (a), (b) and (c); at least one Asn within B′, when B′ is present, is replaced by a structure selected from (a), (b) and (c); at least one Asn within C′, when C′ is present, is replaced by a structure selected from (a), (b) and (c); at least one Asn within D′, when D′ is present, is replaced by a structure selected from (a), (b) and (c); at least one Asn within E′, when E′ is present, is replaced by a structure selected from (a), (b) and (c); at least one Asn within A′, when A′ is present, is replaced by structure (a); at least one Asn within B′, when B′ is present, is replaced by structure (a); at least one Asn within C′, when C′ is present, is replaced by structure (a); at least one Asn within D′, when D′ is present, is replaced by structure (a); at least one Asn within E′, when E′ is present, is replaced by structure (a); at least one Asn within A′, when A′ is present, is replaced by structure (b); at least one Asn within B′, when B′ is present, is replaced by structure (b); at least one Asn within C′, when C′ is present, is replaced by structure (b); at least one Asn within D′, when D′ is present, is replaced by structure (b); at least one Asn within E′, when E′ is present, is replaced by structure (b); at least one Asn within A′, when A′ is present, is replaced by structure (c); at least one Asn within B′, when B′ is present, is replaced by structure (c); at least one Asn within C′, when C′ is present, is replaced by structure (c); at least one Asn within D′, when D′ is present, is replaced by structure (c); and least one Asn within E′, when E′ is present, is replaced by structure (c)

Also disclosed a polypeptide produced by the hydrolysis of structure (a), (b), and/or (c) within any of the aforementioned polypeptides.

In certain cases the peptides include either one or two or more contiguous negatively charged amino acids (e.g., Asp or Glu) or one or two or more contiguous positively charged residues (e.g., Lys or Arg) or one or two or more contiguous positively or negatively charged amino acids at the carboxy terminus. In these cases all of the flanking amino acids at the carboxy terminus are either positively or negatively charged. In other cases the carboxy terminal charged amino acids are preceded by a Leu. For example, any of the following amino acid sequences can be added to the carboxy terminus of the peptide: Asp; Asp Lys; Lys Lys Lys Lys Lys Lys; Asp Lys Lys Lys Lys Lys Lys; Leu Lys Lys; and Leu Asp. It is also possible to simply add Leu at the carboxy terminus.

Also described are nucleic acid molecules comprising a nucleotide sequences encoding an aforementioned polypeptide. The nucleic molecules can optionally include transcription and translation control sequences operably linked to the polypeptide encoding sequences such that the nucleic acid molecule can direct the expression of the polypeptide within a prokaryotic cell, for example, E. coli. The nucleic acid molecule can be within a vector that allows replication in a prokaryotic cell and includes a slectable marker. Also described are cells (e.g., prokaryotic cells) harboring the nucleic acid molecule and cells harboring the vector. Such cells are commonly referred to a recombinant cells.

Compositions, including pharmaceutical compositions, can include at least one such polypeptide or can include at least two (three, four or more) such polypeptides which are different. In the compositions containing two or more such polypeptides the polypeptides can be separate or they can be covalently direct linked, e.g., by a peptide bond or a linker or they can be indirectly linked. For example, two such polypeptide sequences can be contained within a larger polypeptide and the two polypeptide sequences can be separated by other polypeptide sequences.

Variant Polypeptides

The disclosure includes variant polypeptides that can include one, two, three, four, or five or more (e.g., 6, 7, 8, 9, 10, 11, 12, 13, 14, or 15) amino acid substitutions compared to any of the polypeptides described above. The substitution(s) can be conservative or non-conservative. The naturally-occurring amino acids can be substituted by D-isomers of any amino acid, non-natural amino acids, natural and non-natural amino acid analogs, and other groups. A conservative amino acid substitution results in the alteration of an amino acid for a similar acting amino acid, or amino acid of like charge, polarity, or hydrophobicity. At some positions, even conservative amino acid substitutions can reduce the activity of the polypeptide. A conservative substitution can substitute a naturally-occurring amino acid for a non-naturally-occurring amino acid. Among the naturally occurring amino acid substitutions generally considered conservative are:

| For | Replace |
|---|---|
| Ala | Gly, Cys, |
| Arg | Lys, His |
| Asn | Asp, Glu, |
| Asp | Asn, Glu, |
| Cys | Met, Thr, |
| Gln | Asn, Glu, |
| Glu | Asp, Asn, |
| Gly | Ala |
| His | Lys, Arg |
| Ile | Val, Leu, |
| Leu | Val, Ile, |
| Lys | Arg, His |
| Met | Ile, Leu, Val |
| Phe | Tyr, His, |
| Pro | |
| Ser | Thr, Cys, |
| Thr | Ser, Met, |
| Trp | Phe, Tyr |
| Tyr | Phe, His |
| Val | Leu, Ile, |

In general, “identity,” as used herein, refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

The peptides of the disclosure can be present with a counterion. Useful counterions include salts of: acetate, benzenesulfonate, benzoate, calcium edetate, camsylate, carbonate, citrate, edetate (EDTA), edisylate, embonate, esylate, fumarate, gluceptate, gluconate, glutamate, glycollylarsanilate, hexylresorcinate, iodide, bromide, chloride, hydroxynaphthoate, isethionate, lactate, lactobionate, estolate, maleate, malate, mandelate, mesylate, mucate, napsylate, nitrate, pantothenate, phosphate, salicylate, stearate, succinate, sulfate, tartarate, tartrate, hydrochlorate, theoclate, acetamidobenzoate, adipate, alginate, aminosalicylate, anhydromethylenecitrate, ascorbate, aspartate, camphorate, caprate, caproate, caprylate, cinnamate, cyclamate, dichloroacetate, formate, gentisate, glucuronate, glycerophosphate, glycolate, hippurate, fluoride, malonate, napadisylate, nicotinate, oleate, orotate, oxalate, oxoglutarate, palmitate, pectinate, pectinate polymer, phenylethylbarbiturate, picrate, propionate, pidolate, sebacate, rhodanide, tosylate, and tannate.

### Expression Constructs

The present disclosure features expression constructs encoding one or more isolated nucleic acids encoding one or more guanylyl cyclase C (GC-C) activating prohormones (e.g., prohormone forms of guanylin (GN) and/or uroguanylin (UGN)). In general, suitable expression constructs include those capable of entering a target cell (e.g., a prokaryotic and/or eukaryotic cell) and maintaining and expressing one or more of the peptides described herein within the target cell.

Exemplary expression constructs include, but are not limited to, naked DNA constructs, DNA vectors, and viral vectors. Combinations of expression vectors, for example, combinations of naked DNA constructs, DNA vectors, and/or viral vectors, are also useful in the presently described methods.

Expression constructs can include, but are not limited to, one or more of the components detailed below. Such components may be useful, e.g., to target the construct into a target cell and/or promote and/or enhance expression (including transcription and/or translation) of the construct and its components in a target cell. The choice of components to include in a construct can be optimized to, for example, yield the highest expression level of the construct encoded nucleic acid in the target cell. Such components are well known in the art and their use is described, for example, in Current Protocols in Molecular Biology, Ausubel, F. M. et al. (eds.) Greene Publishing Associates, (1989), Sections 9.10-9.14 and other standard laboratory manuals.

Promoter Sequences

Promoter sequences can be used to drive the expression (transcription) of one or more nucleic acid sequences in a target cell. In some cases, one promoter can be operatively linked to one or more nucleic acid sequences encoding one or more of the polypeptides described herein. When one promoter is operatively linked to two or more nucleic acids encoding two or more of the polypeptides described herein, one or more internal ribosome entry site (IRES) elements can be inserted, e.g., between each nucleic acid.

Exemplary promoter sequences that can be used in the expression constructs of the present disclosure include, e.g., promoter sequences capable of driving gene expression in target cell.

Promoters can be selected based on the relative strength of the promoter in a target cell (e.g., a specific prokaryotic cell such as Escherichia coli or a eukaryotic cell). The sequence and relative strengths of common promoters are well known in the art. Expression constructs can include one or more prokaryotic promoter sequences operatively linked to a nucleic acid encoding a peptide described herein (e.g., one promoter per peptide). Expression constructs containing more than one promoter may contain multiple copies of the same promoter and/or different promoters. The choice of promoter or promoter combinations can be optimized to yield the highest expression level of the peptide of interest in the target cell. Commonly used prokaryotic promoter sequences that may be useful in the present disclosure include, but are not limited to, T7, T3, and T5 bacteriovirus promoter sequences. Methods for selecting strong bacterial promoters are described, e.g., by Sakanyan (E.P. 1, 441, 036). Other exemplary promoters useful for expression in E. coli are described, e.g., by O'Neill (O'Neill, Jour. Biol. Chem., 264:5522-5530, 1989).

Alternatively or in addition, an expression construct can include one or more eukaryotic promoter sequences. A database of well known eukaryotic promoters can be accessed, for example at world wide web address epd.isb-sib.ch. The sequence and relative strengths of common eukaryotic promoters are well known in the art.

In some cases, one or more promoter sequences can be operatively linked to one or more of the polypeptide encoding nucleotide sequences described herein.

IBES Elements

Internal ribosomal entry site (IRES) elements are nucleotide sequences that allow for cap-independent translation initiation in the middle of a messenger RNA (mRNA). IRES elements can be usefully inserted, e.g., into bicistronic expression constructs to support the expression (translation) of a second nucleic acid. IRES elements can be added intercistronicly to a construct to confer internal initiation of translation of an mRNA product independent of a 5′ cap. Exemplary IRES elements include those present in, e.g., picornavirus, poliovirus, encephalomyocarditis virus, foot-and-mouth disease virus, flavivirus, hepatitis C virus, pestivirus, classical swine fever virus, retrovirus, murine leukemia virus, lentivirus, simian immunodeficiency virus, insect RNA virus, and cricket paralysis virus.

Antibiotic Resistance Genes

Antibiotic resistance genes are routinely included in expression constructs. Inclusion of the antibiotic to which the gene confers resistance ensures that only bacteria containing the expression construct replicate. Commonly used antibiotic resistance genes include, but are not limited to, genes conferring resistance to, for example, ampicillin, kanamycin, tetracycline, chloramphenicol, Zeocin3, G418, and gentamycin. The nucleotide sequences and use of such genes are known in the art.

Shine-Dalgarno Sequence

Shine-Dalgarno Sequences are useful for promoting efficient translation in prokaryotic cells. Shine-Dalgarno Sequences are typically located 6-7 nucleotides upstream from a start codon. The Shine-Dalgarno nucleotide sequence is known in the art.

Multiple Cloning Site

Multiple cloning sites typically contain several well characterized restriction endonuclease sites to facilitate insertion of DNA fragments into an expression construct. One or more multiple cloning sites may be usefully added to a expression construct.

Affinity Tags

Affinity tags can be used to isolate and/or purify expressed polypeptides. Nucleotide sequences encoding one or more tags can be inserted or positioned, e.g., in frame, onto the amino and/or carboxy-terminals of a nucleotide sequence encoding a protein of interest. Translation of such a construct yields a recombinant fusion protein. Routine methods are available to isolate the expressed fusion protein. Exemplary useful affinity tags include, but are not limited to E. coli maltose E binding protein (MBP), glutathione S-transferase (GST), hexa-histidine (6-His), thioredoxin (TrxA), S-Tag, the Xpress3 epitope, protein A, FLAG tag, hexa-histidine, myc tag and the influenza HA tag.

Affinity Tags

In some instances where affinity tags are utilized, DNA sequence encoding a protease recognition site will be fused between the nucleotide sequence encoding the affinity tag and the nucleotide sequence encoding the polypeptide of interest. One or more protease recognition sites can be included, e.g., to allow proteolytic cleavage of a purification tag from a protein of interest, e.g., following isolation and purification of the fusion protein. Exemplary protease recognition sites include but are not limited to, the PreScission3 protease recognition site (Leu-Glu-Val0-Leu-Phe-Gln-Gly-Pro (SEQ ID NO: 25)), the enterokinase protease recognition site (Asp-Asp-Asp-Asp-Lys (SEQ ID NO:26), the factor Xa protease recognition site (Ile-X₁-Gly-Arg, where X₁ is Glu or Asp (SEQ ID NO:27)), the thrombin protease recognition site (Leu-Val-Pro-Arg-Gly-Ser (SEQ ID NO:28)), and the TEV protease recognition site (Glu-Asn-Leu-Tyr-Phe-Gln-Gly (SEQ ID NO:29)). Protease recognition sites and proteolytic cleavage methods are described, for example, by LaVallie et al. (Enzymatic and Chemical Cleavage of Fusion Proteins, In Current Protocols in Molecular Biology. Page 16.4.5-16.4.17, John Wiley and Sons, Inc., New York, N.Y.).

In some cases, useful expression constructs can include one or more components selected from the group consisting of an internal ribosomal entry site (IRES) element, a polyadenylation signal, a Kozak Consensus Sequence, an enhancer element, one or more heterologous and synthetic introns, and one or more signal sequences.

In some cases, the polypeptides described herein can be produced (e.g., expressed) using, for example, a DNA vector. Such vectors can include a bacterial or bacteriophage DNA such as bacteriophage lambda or M13 and derivatives thereof. DNA vectors are known in the art and typically are circular, double stranded, DNA molecules that include at least one promoter sequence that facilitates expression (transcription and translation) of the DNA vector and its components and/or one or more nucleic acids that have been introduced into the vector in a target cell.

In some cases, the polypeptides described herein can be expressed using pET32b (Novagen). Other exemplary useful expression vectors include, but are not limited to, for example pGEX (Pharmacia Biotech Inc; Smith, D. B. and Johnson, K. S. (1988) Gene, 67:31-40), pMAL (New England Biolabs, Beverly, Mass.) and pRIT5 (Pharmacia, Piscataway, N.J.) which fuse glutathione S-transferase (GST), maltose E binding protein, and protein A, respectively. Other useful vectors are commercially available from, e.g., Novagen, Invitrogen, Promega, Pharmacia, New England Biolabs, and GE Healthcare

In some cases, uptake of an expression vector into a target cell can be facilitated (e.g., improved) by combining the DNA vector with, for example, a cationic lipid, and forming a DNA complex.

Methods of using the polypeptides described herein are described by Currie et al., (Publication No. WO2007/002971).

Polypeptide Production

As described herein, amino acid sequences (e.g., polypeptides) can be treated with cyanogen bromide to produce a polypeptide that is capable of binding to and/or activating the GC-C receptor. These methods generally require the introduction of one or more methionine residues (e.g., one, two, three, or more methionine residues) at one or more locations within an amino acid chain where cleavage is required. For example, one or more methionine residues can be introduced at a location within a single amino acid sequence that results in the generation of two or more separate amino acid sequences. In some cases, one or more of these resulting amino acid sequences will be capable of binding to and/or activating the GC-C receptor.

One or more methionine residues can be introduced into a nucleotide sequence by chemically synthesizing a nucleotide sequence encoding a methionine codon (ATG) at the desired position. Alternatively or in addition, one or more codons encoding methionine can be introduced into a nucleotide sequence at the desired position using a in vitro mutagenesis kit, for example the QuikChange® Site Directed Mutagenesis kit (Stratagene). Alternatively or in addition, an amino acid sequence can be chemically synthesized with one or more methionine residues inserted at the desired position.

Nucleic acid sequences encoding one or more of the polypeptides described herein can be inserted into an expression construct using, e.g., standard molecular biological cloning and/or subcloning techniques, e.g., polymerase chain reaction (PCR), restriction enzyme digestion, agarose gel electrophoresis, DNA purification (e.g., using agarose gel electrophoresis, phenol:chloroform extraction and/or commercially available DNA purification kits), and DNA ligation. Required clones (e.g., encoding nucleic acids encoding polypeptides of interest) can be obtained and introduced into a cell, e.g., an E. coli cell. These E. coli cells can then be used to produce further clones and can be stored as glycerol stocks. Alternatively or in addition, isolated expression constructs can be stored in a suitable buffer (e.g., TE buffer containing 10 mM Tris-Cl, pH 7.5 and 1 mM EDTA). Requied clones can be verified using sequence analysis. Such expression constructs can be used to express one or more of the polypeptides described herein in a cell.

In some cases, one or more of the polypeptides described herein can be expressed, for example, in prokaryotic or eukaryotic cells. In some cases, the polypeptides can be expressed in E. coli, insect cells (e.g., using baculovirus expression vectors), yeast cells, or mammalian cells (e.g., CHO or COS cells). Suitable host cells are discussed further in Goeddel, (1990) Gene Expression Technology: Methods in Enzymology 185, Academic Press, San Diego, Calif. When expressing a polypeptide in a host cell it can be desirable to adapt the coding sequence of the peptide for the codon bias of the host cell. Alternatively or in addition, expression vectors can be transcribed and translated in vitro, for example using T7 promoter regulatory sequences and T7 polymerase. Alternatively or in addition, polypeptides can be chemically synthesized.

Cyanogen bromide is a chemical used to hydrolyze peptide bonds C-terminal to methionine residues in peptides and proteins (Gross et al., Biochemistry, 6:745-748, 1967 and Kaiser and Metzka, Anal. Biochem., 266:1-8, 1999). One of skill in the art will appreciate that insertion of a methionine residue into a sequence will result in the cleavage of that sequence at a point immediately following the inserted methionine, following treatment with cyanogen bromide. Insertion of a methionine into a peptide sequence that does not encode any other methionine sequences will yield two peptides, following treatment with cyanogen bromide. The presence or insertion of multiple methionine residues in a sequence will yield multiple peptides, following treatment with cyanogen bromide. In some cases, a methionine residue can be inserted between the pro and mature sequences of the peptides described herein. Alternatively or in addition, a polypeptide can be modified by removing (e.g., deleting or substituting, e.g., conservative substitutions) one or more methionine residues, e.g., to prevent cleavage of the polypeptide. In some cases, such a modification will not alter the ability of the polypeptide to bind to and/or activate the GC-C receptor.

In some cases, the polypeptides described herein will be purified (e.g., isolated) from any contaminating material prior to and/or following cyanogen bromide treatment. Such purified polypeptides, e.g., solutions comprising purified polypeptides, can be essentially free of contaminanting material (e.g., material that may be detrimental to the shelf life and/or activity of the polypeptide). For example, powders and/or in a solutions containing purified polypeptides can be 30, 40, 50, 60, 70, 80, 90, 95, 98, 99% and 100% free of contaminating materials.

All the molecular biological techniques required to generate an expression construct described herein are standard techniques that will be appreciated by one of skill in the art. Detailed methods may also be found, e.g., Current Protocols in Molecular Biology, Ausubel, F. M. et al. (eds.) Greene Publishing Associates, (1989), Sections 9.10-9.14 and other standard laboratory manuals.

### Biological Activity Assessment Methods

Biological activity as used herein refers to the ability of a polypeptide to bind to and/or activate a GC-C receptor. Methods for determining, confirming, and/or quantifying the biological activity of one or more of the polypeptides described herein are described below and in the Examples.

Effect on cGMP Levels and Secretion in Ligated Loops Rodent Models

The effect of polypeptides/GC-C agonists described herein on cGMP levels and secretion are studied by injecting polypeptides/GC-C agonists described herein directly into an isolated loop in either wild-type or GC-C KO mice. This is done by surgically ligating a loop in the small intestine of the mouse. The methodology for ligated loop formation is similar to that described in London et al. 1997 Am J Physiol p.G93-105. The loop is roughly centered and is a length of 1-3 cm. The loops are injected with 100 μl of one or more of the above described peptides (5 μg) or vehicle (20 mM Tris, pH 7.5 or Krebs Ringer, 10 mM Glucose, HEPES buffer (KRGH)). Following a recovery time of 90 minutes the loops are excised. Weights are recorded for each loop before and after removal of the fluid contained therein. The length of each loop is also recorded. A weight to length ratio (W/L) for each loop is calculated to determine the effects of the polypeptide/GC-C agonist described herein on secretion.

To determine the effect of the polypeptide/GC-C agonist described herein on cGMP activity, fluid from the loop is collected in ice-cold trichloracetic acid (TCA) and stored at −80° C. for use in an assay to measure cGMP levels in the fluid. Intestinal fluid samples are TCA extracted, and cyclic GMP is measured by EIA according to procedures outlined in the Cayman Chemical Cyclic GMP EIA kit (Cayman Chemical, Ann Arbor, Mich.) to determine cyclic GMP levels in the intestinal fluid of the mouse in the presence of either polypeptide/GC-C agonist described herein or vehicle.

The effects of polypeptides/GC-C agonists described herein on cGMP levels and secretion in ligated loops in female CD rats can also be determined using protocols similar to those described above. In the case of the rat, however four loops of intestine are surgically ligated. The first three loops are distributed equally in the small intestine and the fourth loop is located in colon. Loops are 1 to 3 centimeters, and are injected with 2004 of either polypeptide/agonist described herein (5 μg) or vehicle (Krebs Ringer, 10 mM glucose, HEPES buffer (KRGH)).

Intestinal GC-C Receptor Binding and Activity Assays

The ability of polypeptides, variant polypeptides and other compounds to bind to and activate the intestinal GC-C receptor can be tested using the T84 human colon carcinoma cell line (American Type Culture Collection (Bethesda, Md.).

Briefly, cells are grown to confluency in 24-well culture plates with a 1:1 mixture of Ham's F12 medium and Dulbecco's modified Eagle's medium (DMEM), supplemented with 5% fetal calf serum and are used at between passages 54 and 60.

Monolayers of T84 cells in 24-well plates are washed twice with 1 ml/well DMEM, then incubated at 37° C. for 10 min with 0.45 ml DMEM containing 1 mM isobutylmethylxanthine (IBMX), a cyclic nucleotide phosphodiesterase inhibitor. Test polypeptides (50Tl) are then added and incubated for 30 minutes at 37° C. The media is aspirated and the reaction is terminated by the addition of ice cold 0.5 ml of 0.1N HCl. The samples are held on ice for 20 minutes and then evaporated to dryness using a heat gun or vacuum centrifugation. The dried samples are resuspended in 0.5 ml of phosphate buffer provided in the Cayman Chemical Cyclic GMP EIA kit (Cayman Chemical, Ann Arbor, Mich.). Cyclic GMP is measured by EIA according to procedures outlined in the Cayman Chemical Cyclic GMP EIA kit.

For the binding assay, T84 cell monolayers in 24-well plates are washed twice with 1 ml of binding buffer (DMEM containing 0.05% bovine serum albumin and 25 mM HEPES, pH 7.2), then incubated for 30 min at 37° C. in the presence of mature radioactively labeled E. coli ST polypeptide and the test material at various concentrations. The cells are then washed 4 times with 1 ml of DMEM and solubilized with 0.5 ml/well 1N NaOH. The level of radioactivity in the solubilized material is then determined using standard methods.

Murine Gastrointestinal Transit (GIT) Assay

In order to determine whether a test compound or a polypeptide, increases the rate of gastrointestinal transit, the test compound can be tested in the murine gastrointestinal transit (GIT) assay (Moon et al. Infection and Immunity 25:127, 1979). In this assay, charcoal, which can be readily visualized in the gastrointestinal tract is administered to mice after the administration of a test compound. The distance traveled by the charcoal is measured and expressed as a percentage of the total length of the colon.

Mice are fasted with free access to water for 12 to 16 hours before the treatment with polypeptide or control buffer. The polypeptides are orally administered at 1 μg/kg-1 mg/kg of polypeptide in buffer (20 mM Tris pH 7.5) seven minutes before being given an oral dose of 5% Activated Carbon (Aldrich 242276-250G). Control mice are administered buffer only before being given a dose of Activated Carbon. After 15 minutes, the mice are sacrificed and their intestines from the stomach to the cecum are dissected. The total length of the intestine as well as the distance traveled from the stomach to the charcoal front is measured for each animal and the results are expressed as the percent of the total length of the intestine traveled by the charcoal front. Results are reported as the average of 10 mice±standard deviation. A comparison of the distance traveled by the charcoal between the mice treated with polypeptide versus the mice treated with vehicle alone is performed using a Student's t test and a statistically significant difference is considered for P<0.05. Positive controls for this assay may include commercially available wild-type ST polypeptide (Sigma-Aldrich, St Louis, Mo.) and Zelnorm®, a drug approved for IBS that is an agonist for the serotonin receptor 5HT4.

Similar assays can be performed in other rodents, for example, rats. In addition, GIT assays can be performed and compared in wild-type versus rodents lacking the guanylate cyclase C receptor (GC-C KO), for example, using the GC-C KO mice described in Mann et al 1997 Biochem and Biophysical Research Communications 239:463.

Kd Determination and Binding Assays

To determine the affinity of polypeptides/GC-C agonists described herein for GC-C receptors found in rat intestinal mucosa, a competition binding assay is performed using rat intestinal epithelial cells. Epithelial cells from the small intestine of rats are obtained as described by Kessler et al. (J. Biol. Chem. 245: 5281-5288 (1970)). Briefly, animals are sacrificed and their abdominal cavities exposed. The small intestine is rinsed with 300 ml ice cold saline or PBS. 10 cm of the small intestine measured at 10 cm from the pylorus is removed and cut into 1 inch segments. Intestinal mucosa is extruded from the intestine by gentle pressure between a piece of parafilm and a P-1000 pipette tip. Intestinal epithelial cells are placed in 2 ml PBS and pipetted up and down with a 5 ml pipette to make a suspension of cells. Protein concentration in the suspension is measured using the Bradford method (Anal. Biochem. 72: 248-254 (1976)).

A competition binding assay is performed based on the method of Giannella et al. (Am. J. Physiol. 245: G492-G498) between [¹²⁵I] labeled control polypeptide (e.g. wild-type guanylin, uroguanylin or ST polypeptide) and a polypeptide/GC-C agonist described herein.

The assay mixture contains: 0.5 ml of DME with 20 mM HEPES-KOH pH 7.0, 0.9 mg of the cell suspension listed above, 21.4 fmol [¹²⁵I]-labeled control polypeptide (42.8 pM), and different concentrations of competitor polypeptide/GC-C agonist described herein (0.01 to 1000 nM). The mixture is incubated at room temperature for 1 hour, and the reaction stopped by applying the mixture to GF/B glass-fiber filters (Whatman). The filters are washed with 5 ml ice-cold PBS and radioactivity is measured. Kd is determined. % B/Bo is the percentage of the ratio of radioactivity trapped in each sample (B) compared to the radioactivity retained in a control sample with no cold competitor (Bo).

Similar competition binding assays are performed in intestinal epithelial cells from wild-type and guanylate cyclase C knockout (GC-C KO; Mann et al. 1997 Biochem and Biophysical Research Communications 239:463) mice. Mouse intestinal epithelial cells are prepared identical to that above as for rat intestinal epithelial cells except the cells are homogenized with an Omni homogenizer for 20 seconds on the maximum setting to make a suspension of cells. A competition binding assay is performed identical to that described above between ¹²⁵I labeled polypeptide/GC-C agonist described herein and unlabeled polypeptide/GC-C agonist described herein (competitor).

Pharmacokinetic Property Determination of the Polypeptides Described Herein

Serum samples are extracted from the whole blood of exposed (mice dosed orally or intravenously with polypeptide(s) described herein) and control mice, then injected directly (10 mL) onto an in-line solid phase extraction (SPE) column (Waters Oasis HLB 25 μm column, 2.0×15 mm direct connect) without further processing. The sample on the SPE column is washed with a 5% methanol, 95% dH₂O solution (2.1 mL/min, 1.0 minute), then loaded onto an analytical column using a valve switch that places the SPE column in an inverted flow path onto the analytical column (Waters Xterra MS C8 5 μm IS column, 2.1×20 mm). The sample is eluted from the analytical column with a reverse phase gradient (Mobile Phase A: 10 mM ammonium hydroxide in dH₂O, Mobile Phase B: 10 mM ammonium hydroxide in 80% acetonitrile and 20% methanol; 20% B for the first 3 minutes then ramping to 95% B over 4 min. and holding for 2 min., all at a flow rate of 0.4 mL/min.). At 9.1 minutes, the gradient returns to the initial conditions of 20% B for 1 min. polypeptide is eluted from the analytical column and is detected by triple-quadrapole mass spectrometry (MRM, 764 (+2 charge state)>182 (+1 charge state) Da; cone voltage=30V; collision=20 eV; parent resolution=2 Da at base peak; daughter resolution=2 Da at base peak). Instrument response is converted into concentration units by comparison with a standard curve using known amounts of chemically synthesized polypeptide(s) prepared and injected in mouse plasma using the same procedure.

Similarly, pharmacokinetic properties are determined in rats using LCMS methodology. Rat plasma samples containing the polypeptide are extracted using a Waters Oasis MAX 96 well solid phase extraction (SPE) plate. A 200 μL volume of rat plasma is mixed with 200 μL of ¹³C₉, ¹⁵N-labeled polypeptide in the well of a prepared SPE plate. The samples are drawn through the stationary phase with 15 mm Hg vacuum. All samples are rinsed with 200 μL of 2% ammonium hydroxide in water followed by 200 μL of 20% methanol in water. The samples are eluted with consecutive 100 μL volumes of 5/20/75 formic acid/water/methanol and 100 μL 5/15/80 formic acid/water/methanol. The samples are dried under nitrogen and resuspended in 100 μL of 20% methanol in water. Samples are analyzed by a Waters Quattro Micro mass spectrometer coupled to a Waters 1525 binary pump with a Waters 2777 autosampler. A 40 μL volume of each sample is injected onto a Thermo Hypersil GOLD C18 column (2.1×50 mm, 5 um). polypeptide is eluted by a gradient over 3 minutes with acetonitrile and water containing 0.05% trifluoroacetic acid. The Quattro Micro mass spectrometer is run in multiple reaction monitoring (MRM) mode using the mass transitions of, for example 764>182 or 682>136. Using this methodology, polypeptide is dosed orally and by IV to rats at 10 mg/kg. Pharmacokinetic properties including area under the curve and bioavailability are determined.

Determination of In Vitro Proteolytic Stability

Polypeptide/GC-C agonists described herein are exposed to a variety of in vitro conditions including digestive enzymes and low ph environments designed to simulate gastric fluid. Polypeptide/GC-C agonists described herein are incubated with chymotrypsin, trypsin, pepsin, aminopeptidase, carboxypeptidase A, and simulated gastric fluid (sgf) at ph 1.0. Samples are collected at 0, 3, and 24 h for all conditions except pepsin digestion and the SGF. For the latter two conditions, samples are obtained at 0, 1, and 3 h. Negative control samples are prepared for initial and final time points. A separate, positive activity control is run in parallel for each condition. All samples are analyzed by LC/MS.

Additional methods for confirming and/or determining the biological activity of the peptides described herein are provided by Currie et al., (Publication No. WO2007/002971) and in the Examples.

### EXAMPLES

The disclosure is further described in the following examples, which do not limit the scope of the disclosure described in the claims.

### Example 1

### Design and Construction of DNA Encoding Human Pro-M-Uroguanylin

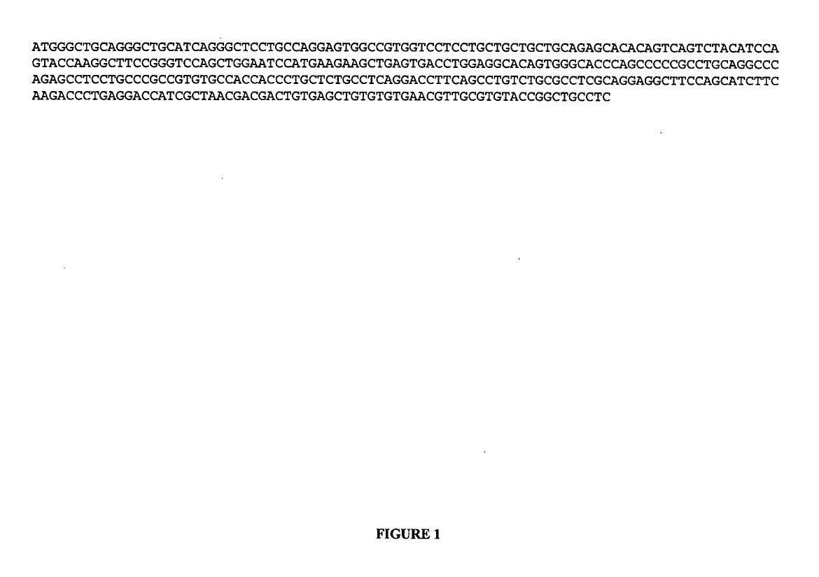

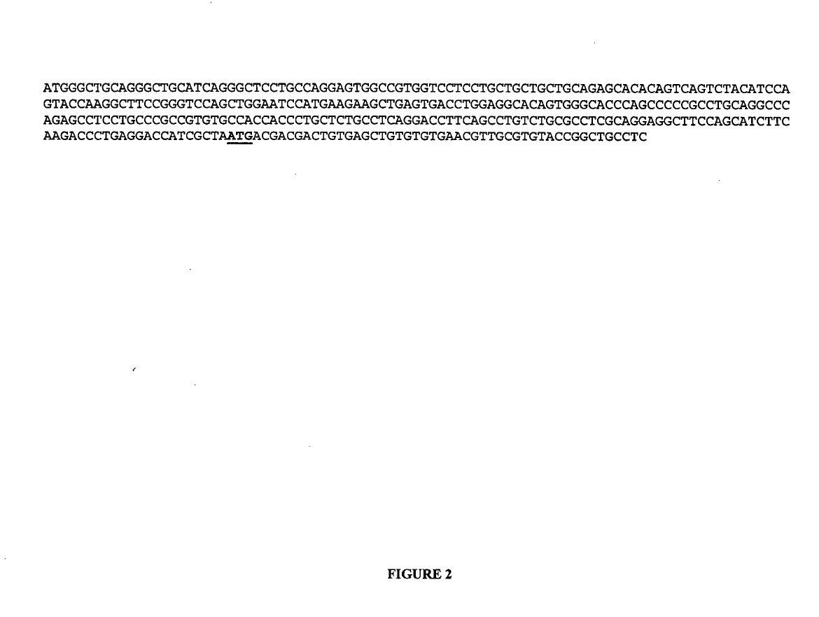

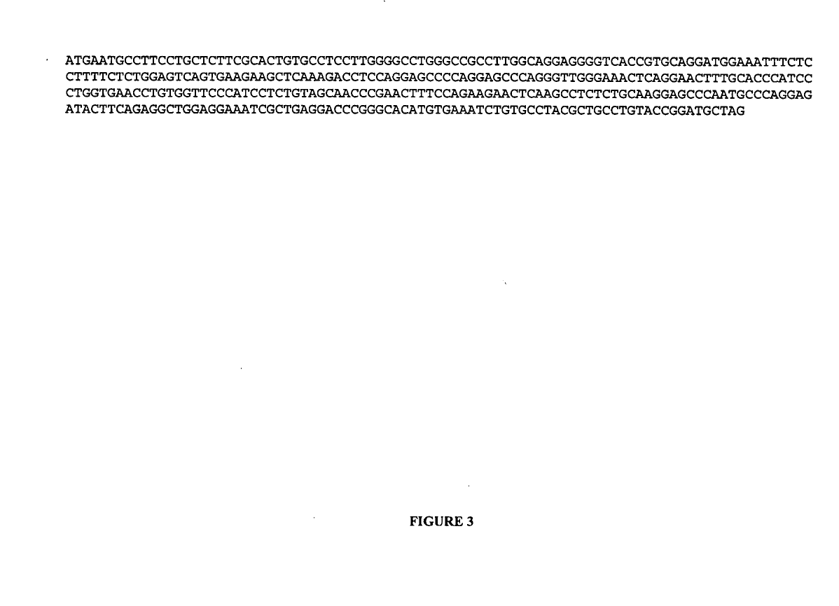

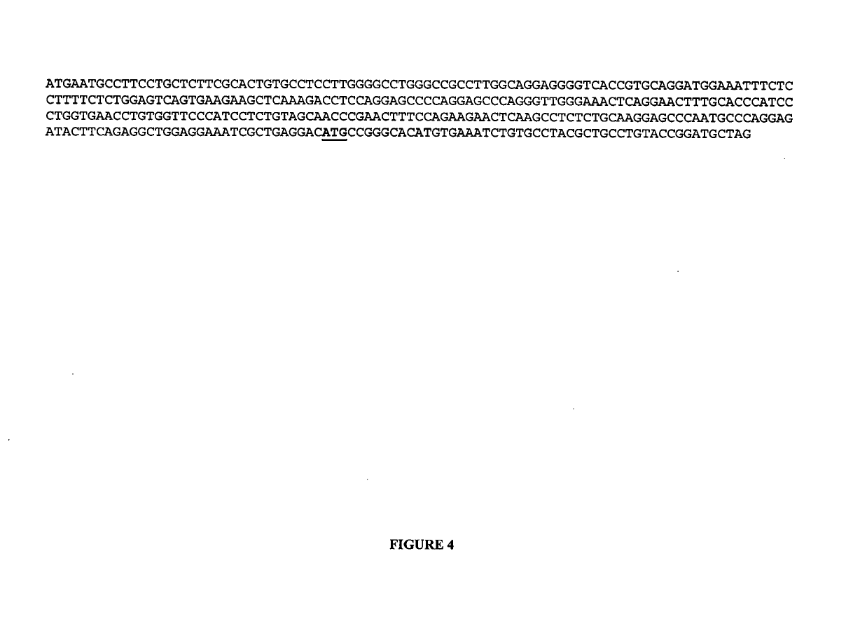

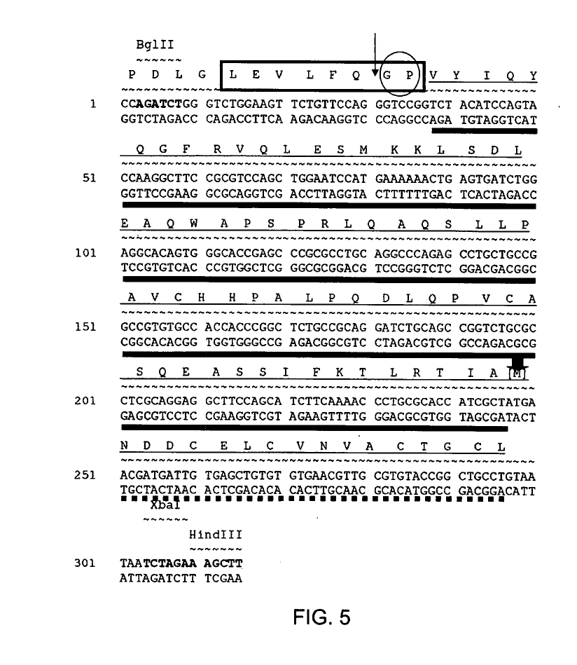

FIGS. 1 and 3 show the unmodified nucleotide and amino acid sequence of human pro-uroguanylin (huProUGN). huProUGN differs from human pro-M-uroguanylin (huProMUGN) because the latter contains a single methionine residue inserted into the huProUGN sequence, as shown in FIGS. 2 and 5. This methionine residue facilitates cyanogen bromide (CNBr) cleavage of the peptide. In huProMUGN, the methionine residue is inserted between the pro and mature uroguanylin sequences. Insertion of the methionine residue at this position is expected to yield pre and mature UGN sequences following CNBr cleavage.

A DNA construct encoding huProMUGN was synthesized by Blue Heron Biotechnology (Bothell, Wash.). As shown in FIG. 5, in addition to the methionine residue described above, huProMUGN includes a nucleotide sequence encoding a PreScission3 protease amino acid recognition site (LEVLFQGP (SEQ ID NO: 25); GE Healthcare) located at the five prime terminus. The PreScission3 site was included to facilitate cleavage of the huProMUGN sequence from any upstream molecules, e.g., fusion proteins.

huProMUGN DNA was then cloned into pUC119 (Vierra and Messing, Methods in Enzymology, 153:3-11, 1997) without the use of a cloning site. BglII and HindIII restriction endonuclease sites were then inserted at the five and three prime terminals of the huProMUGN coding sequence, respectively, and a XbaI restriction enzyme recognition site was inserted close to the three prime end of the huProMUGN DNA sequence. Sequences were validated throughout each of the cloning steps above using sequence analysis. The complete huProMUGN DNA construct and translated amino acid sequence is shown in FIG. 5 and is designated as TP26 (BlueHeron clone CCN 57126).

The huProMUGN coding region was excised from TP26 and cloned into pET32b (Novagen) using BglII and HindIII restriction enzymes to digest TP26 and pET32b. This strategy resulted in the deletion of the enterokinase cleavage present in pET32b, but left the TrxTag, the HisTag, the thrombin site, and the S-tag intact and inframe with the huProMUGN. The resulting vector was designated pTM202.

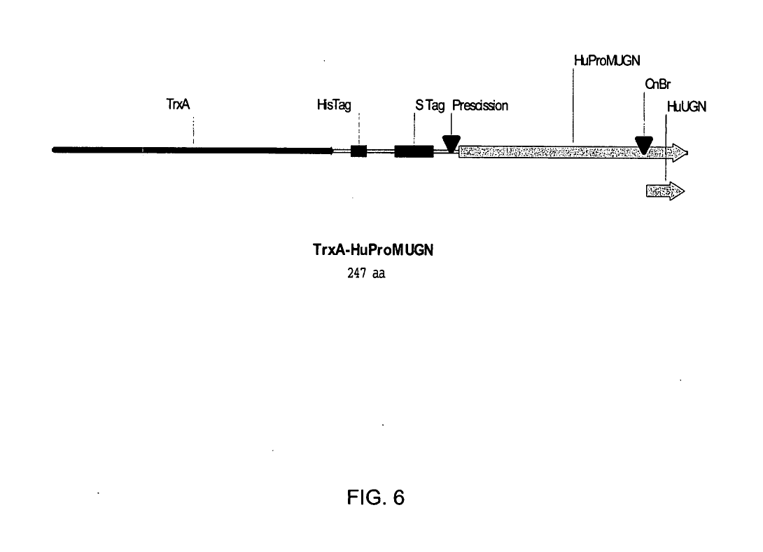

The 247 amino acid Trx-huProMUGN fusion peptide that results from the expression of pTM202 is shown schematically in FIG. 6.

### Example 2

### Recombinant Protein Expression and Purification

pTM202 was transformed into Escherichia coli Origami (DE3), according to the manufacturer′s instructions (Novagen). The resulting cell line was designated pTM202/DE3. A starter culture containing 3 mL Luria-Bertani (LB) broth supplemented with 50 Tg/mL ampicillin was inoculated with pTM202/DE3 and cultured over night at 37° C. The starter culture was then used to inoculate 250 mL of LB supplemented with 50 Tg/mL ampicillin. These cultures were then grown to an OD₆₀₀ of about 0.5 at 37° C., before the incubation temperature was decreased to 25° C. Protein expression was then induced by the addition of isopropyl-beta-D-thiogalactopyranoside (IPTG) to a final concentration of 1 mM. These conditions were maintained for four hours. Cells were then harvested by centrifugation. Cell pellets were frozen overnight at −80° C. The cell pellet was thawed and resuspended in 10 mL buffer A (20 mM Tris-HCL, pH 8.0, 150 mM NaCl) supplemented with 0.2 mg/mL lysozyme and EDTA-free protease inhibitor (Roche Applied Science, Mannheim, Germany) per gram wet cell weight. The cells were then lysed using three 60 s sonication steps, and the soluble fraction was extracted by centrifugation for 45 min at 37,000×g at 4° C. in a Sorvall SA-600 rotor.

His-tagged fusion proteins were purified from the soluble fraction using a TALON metal affinity column. Briefly, the soluble fraction was loaded onto a 2.5 mL bed volume TALON metal affinity column (Clontech, Palo Alto, Calif.) pre-equilibrated with buffer A. The column was then washed with 10 bed volumes of buffer B (20 mM Tris-HCL, pH 8.0, 500 mM NaCl), followed by 3 bed volumes of buffer B containing 5 mM imidazole. The recombinant protein was then eluted with buffer A containing 150 mM imidazole. The eluate was desalted and concentrated using an Amicon Centriprep centrifugal filter unit (Millipore, Billerica, Mass.) to a final volume of 1 mL.

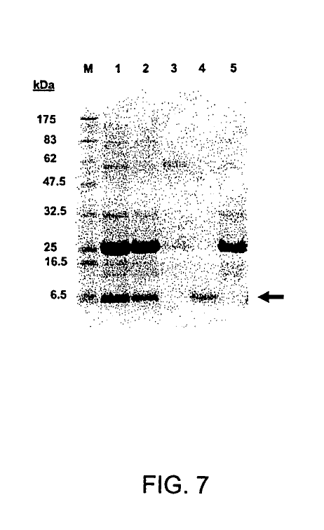

Purified recombinant proteins were then treated with 100 units of PreScission3 protease at 4° C. to remove the upstream fusion proteins (TrxA, His, and S tags). The cleavage reaction was monitored throughout by running aliquots of the digest mixture on an SDS-PAGE gel, as shown in FIG. 7.

Following sufficient digestion, the digest mixture was loaded onto a 250 TL bed volume glutathione sepharose 4B column (GE Healthcare, Uppsala, Sweden) pre-equilibrated with buffer A to remove the PreScission protease. The flow-through was collected and loaded onto the TALON metal affinity column a second time to remove the thioredoxin tag and uncleaved fusion proteins. Purified huProMUGN was collected in the flow-through, as shown in FIG. 6, lane 4.

### Example 3

### Generation of Mature UGN Peptide

The concentration of purified huProMUGN was determined using the Bradford Assay. A 100-fold molar excess of CNBr was then added to 100 TL of protein. Following an overnight incubation at room temperature, the digest mixture was dried at 55° C. and resuspended in 100 TL of 100 mM Tris-HCl, pH 8.0.

The activity of the mature peptide was evaluated by the production of intracellular cGMP using cultured human colon carcinoma T84 cells, as described by Currie et al. (Pro. Natl. Acad. Sci. USA., 89:947-951, 1992). cGMP levels were subsequently measured using a cGMP ELISA kit (Cayman Chemical, Ann Arbor, Mich.) according to the manufacturer's protocol. Synthetic human uroguanylin (Sigma-Aldrich, St. Louis, Mo.) was assayed alongside as a comparison.

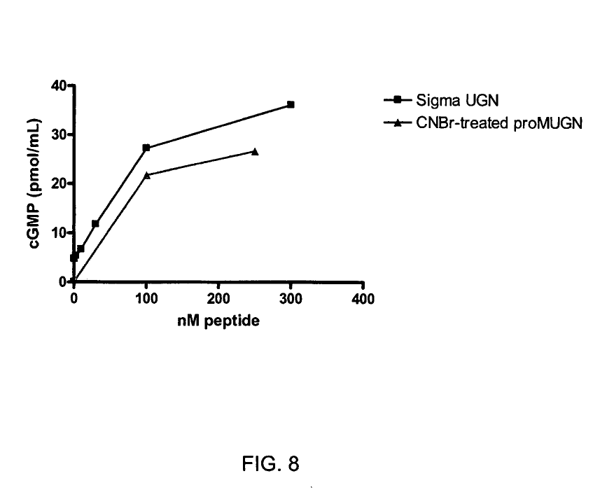

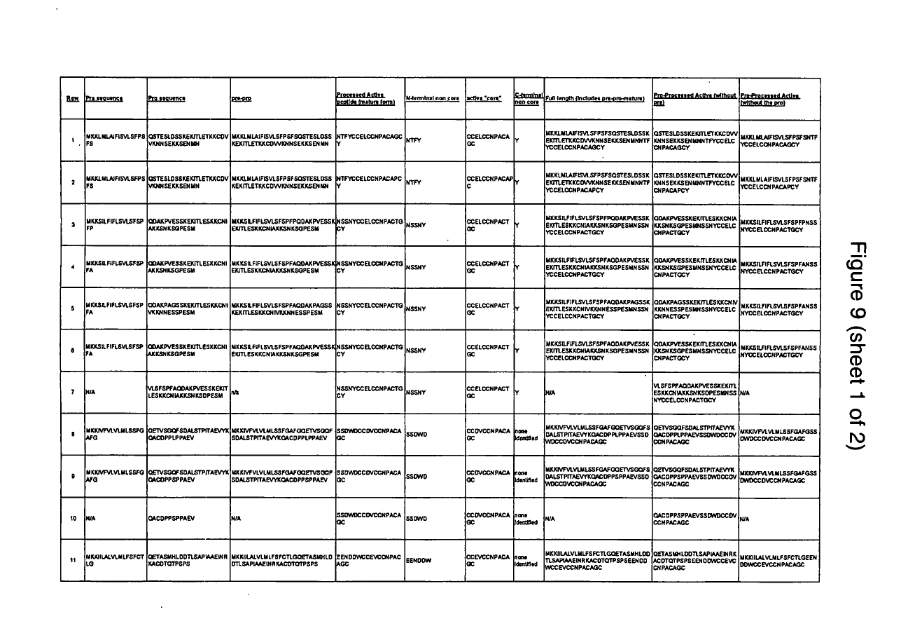

As shown in FIG. 8, cGMP levels produced by the mature UGN peptide were comparable to those levels produced using synthetic human UGN.

### Other Cases

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1-110. (canceled)

111. A nucleic acid molecule comprising a nucleotide sequence encoding a polypeptide comprising the amino acid sequence:
A-B-C-D-E, wherein:
A comprises a GC-C receptor binding polypeptide presequence or is missing
B is one or more methionine residues or is missing;
C comprises a GC-C receptor binding polypeptide prosequence or is missing;
D is one or more methionine residues or is missing; and
E comprises a GC-C receptor binding polypeptide.

112. The nucleic acid molecule according to claim 111, wherein the polypeptide comprises an amino acid sequence selected from:
A-B-C-D-E,
B-C-D-E,
A-C-D-E,
A-B-C-E,
C-D-E,
A-B-E, and
D-E;
wherein:
A comprises a GC-C receptor binding polypeptide presequence;
B is one or more methionine residues;
C comprises a GC-C receptor binding polypeptide prosequence;
D is one or more methionine residues; and
E comprises a GC-C receptor binding polypeptide.

113. The nucleic acid molecule of claim 111, wherein B, when present, is one methionine.

114. The nucleic acid molecule of claim 111, wherein D, when present, is one methionine.

115. The nucleic acid molecule of claim 111, wherein A, when present, comprises a sequence selected from SEQ ID NO:17, SEQ ID NO:18, SEQ ID NO:19, SEQ ID NO:20, SEQ ID NO:21, SEQ ID NO:22, or a presequence depicted in FIG. 9 or FIG. 10.

116. The nucleic acid molecule of claim 111, wherein C, when present, comprises a sequence chosen from a prosequence depicted in FIG. 9 or FIG. 10, SEQ ID NO:11, SEQ ID NO:12, SEQ ID NO:13, or SEQ ID NO:15933.

117. The nucleic acid molecule of claim 111, wherein E comprises a sequence selected from: a processed active peptide (mature) sequence depicted in FIG. 10, SEQ ID NO:9, SEQ ID NO:10, SEQ ID NO:14; SEQ ID NO:199; SEQ ID NO:15933; SEQ ID NO:15, or SEQ ID NO:16.

118. The nucleic acid molecule of claim 111, wherein E comprises a sequence chosen from the group consisting of SEQ ID NOs: 27-33, 34-59, 63-162, and 805.

119. The nucleic acid molecule of claim 118 wherein E is selected from SEQ ID NOs: 63, 64, and 805.

120. The nucleic acid molecule of claim 111 wherein E comprises
E1-E2-E3, wherein
E1 is an N-terminal non-core sequence in FIG. 9, or is missing;
E2 is an active “core” sequence in FIG. 9; and
E3 is a C-terminal non-core sequence in FIG. 9, or is missing.

121. The nucleic acid molecule of claim 120 wherein E is selected from
E1-E2-E3,
E1-E2,
E2-E3, and
E2
wherein
E1 is an N-terminal non-core sequence in FIG. 9;
E2 is an active “core” sequence in FIG. 9; and
E3 is a C-terminal non-core sequence in FIG. 9.

122. The nucleic acid molecule of claim 111 wherein E comprises
E1-E1′-E2-E3, wherein
E1 is an N-terminal non-core sequence in FIG. 9;
E1′ is one or more methionine residues;
E2 is an active “core” sequence in FIG. 9; and
E3 is a C-terminal non-core sequence in FIG. 9.

123. The nucleic acid molecule of claim 122 wherein E1′ is one methionine residue.

124. The nucleic acid molecule of claim 111 wherein E comprises a sequence chosen from a sequence depicted in FIG. 10.

125. The nucleic acid molecule of claim 111 wherein E comprises a sequence selected from SEQ ID NOs:1629-5000; SEQ ID NOs:5001-9000; SEQ ID NOs:9001-13000; and SEQ ID NOs:13001-15933.

126. A nucleic acid molecule comprising a nucleotide sequence encoding a polypeptide comprising the amino acid sequence: Z₂-Z₃, wherein:
Z₂ is one or more methionine residues; and
Z₃ comprises SEQ ID NO:9, SEQ ID NO:10, SEQ ID NO:15, or SEQ ID NO:16.

127. The nucleic acid molecule of claim 126, wherein Z₃ consists of a sequence of SEQ ID NO:16.

128. The nucleic acid molecule of claim 126, wherein the polypeptide further comprises Z₀ or Z₁ or both, wherein:
Z₀ is SEQ ID NO:17, SEQ ID NO:18, SEQ ID NO:19, SEQ ID NO:20, SEQ ID NO:21, SEQ ID NO:22, a pre sequence depicted in FIG. 9, or is missing; and
Z₁ comprises SEQ ID NO:11, SEQ ID NO:12, SEQ ID NO:13, or SEQ ID NO:15933, a pro sequence depicted in FIG. 9, or is missing.

129. The nucleic acid molecule of claim 128, wherein the polypeptide is selected from
Z₁-Z₂-Z₃,
Z₀-Z₂-Z₃,
Z₀-Z₁-Z₂-Z₃,
Z₀-Z₂-Z₁-Z₂-Z₃, and
Z₀-Z₂-Z₁-Z₃,
wherein:
Z₀ is SEQ ID NO:17, SEQ ID NO:18, SEQ ID NO:19, SEQ ID NO:20, SEQ ID NO:21, SEQ ID NO:22, a pre sequence depicted in FIG. 9, or is missing; and
Z₁ comprises SEQ ID NO:11, SEQ ID NO:12, SEQ ID NO:13, or SEQ ID NO:15933, a pro sequence depicted in FIG. 9, or is missing;
Z₂ is one or more methionine residues; and
Z₃ comprises SEQ ID NO:9, SEQ ID NO:10, SEQ ID NO:15, or SEQ ID NO:16.

130. A nucleic acid molecule comprising a nucleotide sequence encoding a polypeptide comprising the amino acid sequence:
A′-B′-Z₂-D′,
wherein:
A′ is an amino acid sequence comprising a pre sequence depicted in FIG. 9, or is missing;
B′ is an amino acid sequence comprising a pro sequence depicted in FIG. 9, or is missing;
Z₂ is one or more methionine residues, and
D′ is an amino acid sequence selected from the group consisting of SEQ ID NOs:63-1629, or an active core sequence depicted in FIG. 9.

131. The nucleic acid of claim 130, where the nucleotide sequence encoding a polypeptide comprising the amino acid sequence selected from:
A′-Z₂-D′,
B′-Z₂-D′, and
Z₂-D′
wherein:
A′ is an amino acid sequence comprising a pre sequence depicted in FIG. 9 or is missing;
B′ is an amino acid sequence comprising a pro sequence depicted in FIG. 9 or is missing;
Z₂ is one or more methionine residues; and
D′ is an amino acid sequence selected from the group consisting of SEQ ID NOs:63-1629, or an active core sequence depicted in FIG. 9.

132. A nucleic acid molecule comprising a nucleotide sequence encoding a polypeptide comprising any of the amino acid sequences of Table 3, wherein
A′ is an amino acid sequence comprising a pre sequence depicted in FIG. 9;
B′ is an amino acid sequence comprising a pro sequence depicted in FIG. 9;
each Z₂ is one or more methionine residues; and
D′ is an amino acid sequence selected from the group consisting of SEQ ID NOs:63-1629, or an active core sequence depicted in FIG. 9.

133. The nucleic acid molecule of claim 132 further comprising amino acid sequences of C′, or E′, or both, wherein C′, if present, is located between Z₂ and D′ and E′, if present, is located adjacent to and following D′.

134. The nucleic acid molecule of claim 133, wherein C′ is an amino acid sequence comprising an amino-terminal non-core sequence depicted in FIG. 9 or E′ is an amino acid sequence comprising an carboxy-terminal non-core sequence depicted in FIG. 9.

135. The nucleic acid molecule of claim 132, wherein D′ is selected from SEQ ID NOs: 63, 64, and 805.

136. A nucleic acid molecule comprising a nucleotide sequence encoding a polypeptide comprising an amino acid sequence selected from SEQ ID NOs: 7 and 8.

137. A nucleic acid molecule comprising a nucleotide sequence selected from SEQ ID NOs: 24, 2, and 4.

138. The nucleic acid molecule of claim 136, wherein the polypeptide comprises an affinity tag located at the amino-terminus and/or the carboxy-terminus of the polypeptide.

139. The nucleic acid molecule of claim 138, wherein the polypeptide and the affinity tag are adjacent to each other.

140. The nucleic acid molecule of claim 138, wherein the polypeptide and the affinity tag are separated by a protease recognition site.

141. The nucleic acid molecule of claim 137, further comprising a nucleic acid sequence encoding an affinity tag located at the 5′ or 3′ terminus of the nucleic acid molecule.

142. The nucleic acid molecule of claim 141, further comprising a nucleic acid sequence encoding a protease recognition site.

143. An expression vector comprising a nucleic acid molecule of claim 111.

144. The expression vector of claim 143, wherein the nucleic acid molecules is operably linked to a promoter capable of driving expression of the nucleic acid molecule in a cell.

145. The expression vector of claim 144, further comprising an internal ribosome entry site.

146. The expression vector of claim 143, wherein the expression vector is a DNA vector.

147. The expression vector of claim 143, wherein the expression vector is pET32b.

148. A polypeptide encoded by a nucleic acid molecule of claim 132.

149. A recombinant cell comprising a nucleic acid molecule of claim 111.

150. A recombinant cell comprising the expression vector of claim 143.

151. A method of making a polypeptide capable of binding to and/or activating the guanylate cyclase (GC-C) receptor, the method comprising obtaining a polypeptide of claim 148 and cleaving the polypeptide at the carboxy terminal to one or more methionine residues.

152. The method of claim 151, wherein the step of cleaving the polypeptide comprises contacting the polypeptide with cyanogen bromide.

153. The method of claim 151, wherein the polypeptide capable of binding to and/or activating the guanylate cyclase (GC-C) receptor is purified.
